(19) United States
(12) Patent Application Publication
(10) Pub. No.: US 2025/0049578 A1
(43) Pub. Date: Feb. 13, 2025

(54) FACET FIXATION SYSTEMS AND METHOD

(71) Applicant: SYNERGY SPINE SOLUTIONS INC., Kitchener (CA)

(72) Inventors: Cortny ROBISON, Salt Lake City, UT (US); Josh BUTTERS, Erie, CO (US); Bjorn RINDAL, Erie, CO (US); Neil DUGGAL, London (CA); Joe MORREALE, Westminster, CO (US); Hao WANG, Hamilton (CA)

(21) Appl. No.: 18/800,020

(22) Filed: Aug. 10, 2024

Related U.S. Application Data
(60) Provisional application No. 63/532,364, filed on Aug. 12, 2023.

(51) Int. Cl. A61F 2/44; A61B 17/16; A61F 2/30; A61F 2/46

(52) U.S. Cl. CPC A61F 2/4405; A61B 17/1671; A61F 2/30749; A61F 2/4611; A61F 2002/3092; A61F 2002/3093; A61F 2002/30985

(57) ABSTRACT

A system may be configured for facet fixation and may include a cap configured to span a vertebral facet joint. The cap may include a distal surface configured to seat against a bone proximate the vertebral facet joint. The system may also include a fastener configured to secure the cap to the vertebral facet joint. The cap may include an indicator configured to move from a first position to a second position in response to engagement of the distal surface with the bone.

### CROSS-REFERENCE TO RELATED APPLICATIONS

The present disclosure claims the benefit of U.S. Provisional Patent Application No. 63/532,364, which was filed on Aug. 12, 2023, entitled FACET FIXATION SYSTEMS AND METHOD, which is incorporated by reference as though set forth herein in its entirety.

### TECHNICAL FIELD

The present disclosure relates to the field of systems, devices, and methods for bone fixation. More specifically, the disclosure relates to the field of systems, devices, and methods for the stabilization of spinal elements through facet fixation.

### BACKGROUND

Facet joints in the spine play a crucial role in providing stability and flexibility, but they can be compromised by degenerative changes, trauma, or diseases, leading to pain and restricted movement. Facet fixation devices are typically implanted as a minimally invasive means of posteriorly stabilizing the spine. Biomechanical studies have shown that facet screw systems perform similarly or better than pedicle screw systems. Many commercially available facet screws require a complex and timely oblique fluoroscopic technique to achieve the proper placement trajectory.

The development of an efficient and accurate facet fixation system may represent a significant advancement in the treatment of spinal disorders. By addressing the inherent challenges associated with commercially available methods, a novel system may improve surgical outcomes, enhance patient safety, and optimize the overall process of facet joint stabilization. For these reasons, there is a need for a facet fixation system that may enable facet fixation implants to be more efficiently and accurately implanted.

### SUMMARY

The various systems and methods of the present disclosure have been developed in response to the present state of the art, and in particular, in response to the problems and needs in the art that have not yet been fully solved by currently available facet fixation systems and methods.

In some embodiments, a system may be configured for facet fixation and may include a cap configured to span a vertebral facet joint. The cap may include a distal surface configured to seat against a bone proximate the vertebral facet joint. The system may also include a fastener configured to secure the cap to the vertebral facet joint. The cap may include an indicator configured to move from a first position to a second position in response to engagement of the distal surface with the bone.

In the system of any preceding paragraph, the cap and the indicator may be integrally manufactured using an additive manufacturing process.

In the system of any preceding paragraph, the fastener and the cap may be configured so that the fastener is captive relative to the cap.

In the system of any preceding paragraph, the cap may include a plurality of recesses configured to receive a guiding instrument.

In the system of any preceding paragraph, the cap may include a lattice structure configured to promote bone growth into the cap.

In the system of any preceding paragraph, the cap may further include a rod portion configured to couple the facet fixation system with a pedicle screw system.

In the system of any preceding paragraph, the cap may further include a rod portion configured to couple the facet fixation system with a laminar hook.

In the system of any preceding paragraph, the system may further include a tulip configured to receive a rod. The rod may rigidly couple the facet fixation system with a pedicle screw system.

In the system of any preceding paragraph, the system may further include a tulip configured to receive a rod portion of a laminar hook. The rod portion may rigidly couple the facet fixation system with the laminar hook.

In the system of any preceding paragraph, the system may further include a reamer configured to prepare a bone surface for implantation of a facet fixation device. The reamer may include a cutting head having a plurality of cutting edges, wherein the cutting head may be configured to cut in a clockwise direction and a counter-clockwise direction.

In the system of any preceding paragraph, the system may further include a dilator configured to dilate soft tissue, the dilator may have an inside diameter eccentric to an outside diameter.

In the system of any preceding paragraph, the fastener may include a porous surface configured promote bone growth into the fastener.

In the system of any preceding paragraph, the fastener may include a cylindrical screw head and the cap may include an aperture, offset from a geometric center of the cap. The aperture may be configured to rotatably receive the cylindrical screw head.

In some embodiments, a system may be configured for facet fixation and may include a cap configured to span a vertebral facet joint, a fastener configured to secure the cap to the vertebral facet joint, an inserter configured to engage the cap, and a lock that may be movable, relative to the inserter, between a locked position, in which the cap may not be disengageable from the inserter, and an unlocked position, in which the cap may be disengageable from the inserter.

In the system of any preceding paragraph, the cap may include a distal surface configured to seat against a bone proximate the vertebral facet joint and an indicator may be configured to move from a first position to a second position in response to engagement of the distal surface with the bone.

In the system of any preceding paragraph, the cap may include a lattice structure configured to promote bone growth into the cap.

In the system of any preceding paragraph, the system may further include a dilator configured to dilate soft tissue. The dilator may include an inside diameter eccentric to an outside diameter.

In the system of any preceding paragraph, the system may further include a reamer configured to prepare a bone surface for implantation of a facet fixation device. The reamer may include a cutting head including a plurality of cutting edges. The cutting head may be configured to cut in a clockwise direction and a counterclockwise direction.

In some embodiments, a system may be configured for facet fixation and may include a cap configured to span a vertebral facet joint. The cap may include a distal surface configured to seat against a bone proximate the vertebral facet joint, and a proximal surface facing proximally. The system may also include a fastener configured to secure the cap to the vertebral facet joint. The distal surface of the cap may include a first lattice structure configured to promote bone growth into the cap.

In the system of any preceding paragraph, the proximal surface may include a second lattice structure configured to promote bone growth into the proximal surface, wherein the second lattice structure is spaced apart from the first lattice structure.

In the system of any preceding paragraph, the cap may include a hollow interior in communication with the second lattice structure and the first lattice structure such that bone growth may occur through the cap, from the second lattice structure to the first lattice structure, after implantation of the cap.

In the system of any preceding paragraph, the system may further include an inserter configured to engage the cap. The cap may include a load-carrying portion within which the first lattice structure may be absent, and the inserter may be configured to engage the load-carrying portion to permit insertion of the cap into the bone without transmitting force through the first lattice structure.

In the system of any preceding paragraph, the inserter may further include a recessed portion. The recessed portion may be configured to receive the cap and the fastener and may be configured to prevent the fastener from disengaging from the cap during insertion of the cap.

In the system of any preceding paragraph, the system may further include a dilator configured to dilate soft tissue. The dilator may include an inside diameter eccentric to an outside diameter.

In the system of any preceding paragraph, the system may further include a reamer configured to prepare a bone surface. The reamer may include a cutting head including a plurality of cutting edges. The cutting head may be configured to cut in a clockwise direction and a counter-clockwise direction.

These and other features and advantages of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through the use of accompanying drawings.

### BRIEF DESCRIPTION OF THE DRA WINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings in which:

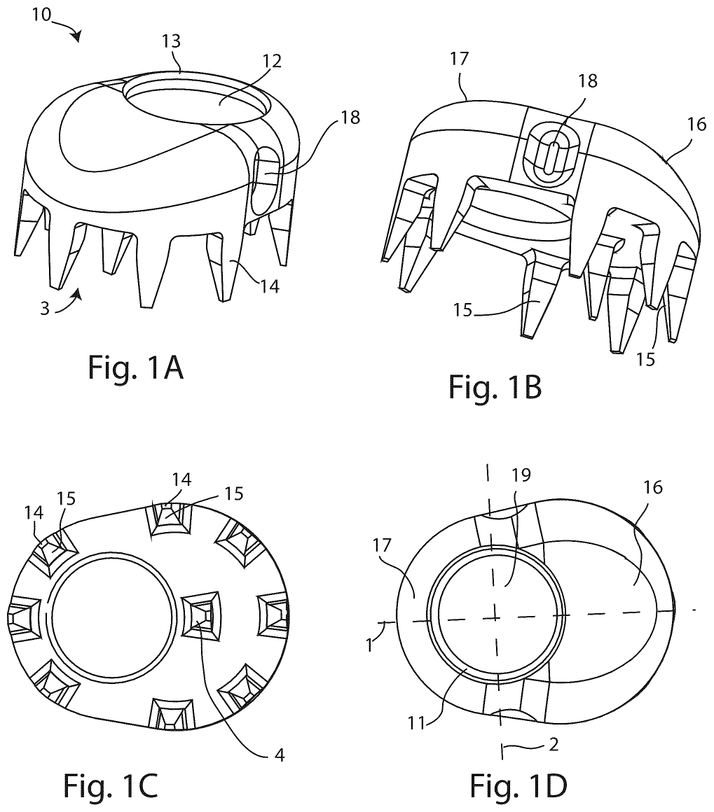

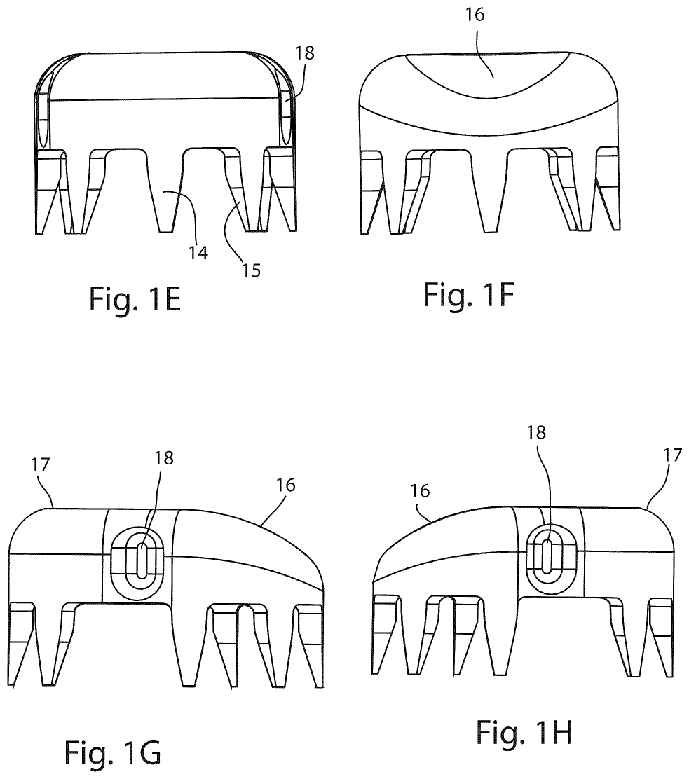

FIG. 1A is a top perspective view of a cap in accordance with an embodiment of the present disclosure.

FIG. 1B is a bottom perspective view of the cap of FIG. 1A.

FIG. 1C is a bottom view of the cap of FIG. 1A.

FIG. 1D is a top view of the cap of FIG. 1A.

FIG. 1E is a back view of the cap of FIG. 1A.

FIG. 1F is a front view of the cap of FIG. 1A.

FIG. 1G is a left-side view of the cap of FIG. 1A.

FIG. 1H is a right-side view of the cap of FIG. 1A.

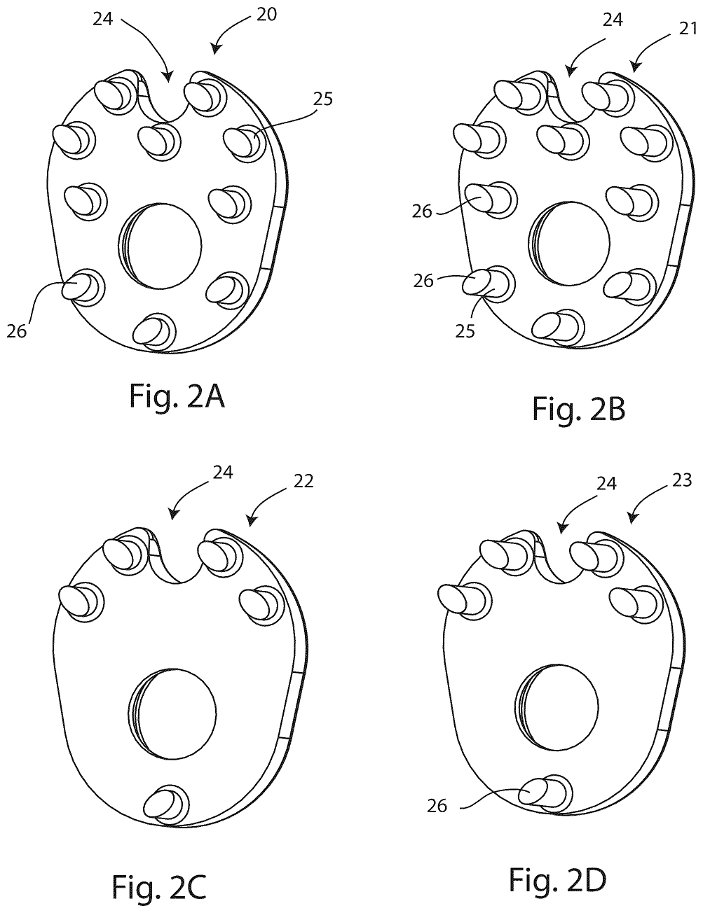

FIG. 2A is a bottom perspective view of a cap in accordance with an embodiment of the present disclosure.

FIG. 2B is a bottom perspective view of the cap of FIG. 2A with longer teeth according to an embodiment of the present disclosure.

FIG. 2C is a bottom perspective view of a cap in accordance with an embodiment of the present disclosure.

FIG. 2D is a bottom perspective view of the cap of FIG. 2C with longer teeth according to an embodiment of the present disclosure.

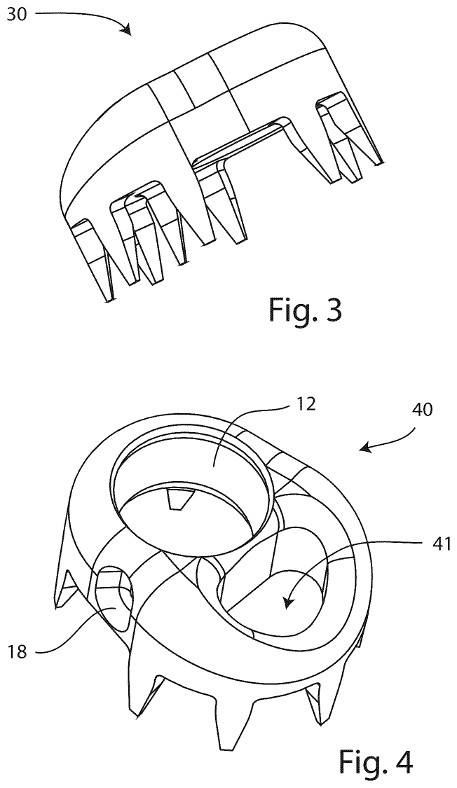

FIG. 3 is side perspective view of a cap in accordance with an embodiment of the present disclosure.

FIG. 4 is top perspective view of a cap in accordance with an embodiment of the present disclosure.

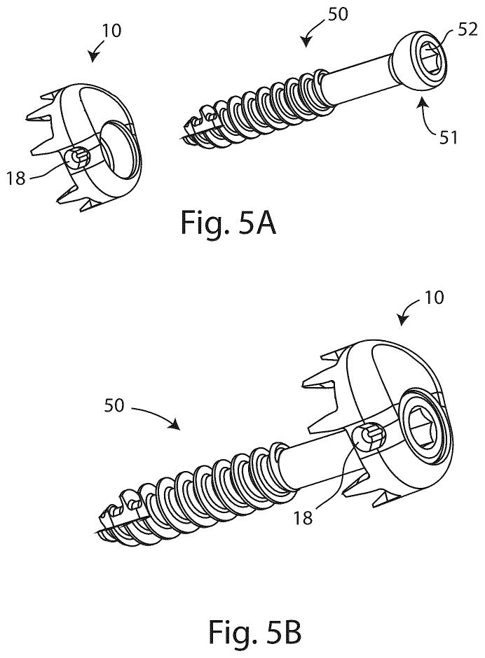

FIG. 5A is a pre-assembled perspective view of an implant including the cap of FIG. 1A and a fastener according to an embodiment of the present disclosure.

FIG. 5B is an assembled perspective view of the cap of FIG. 1A and the fastener of FIG. 5A.

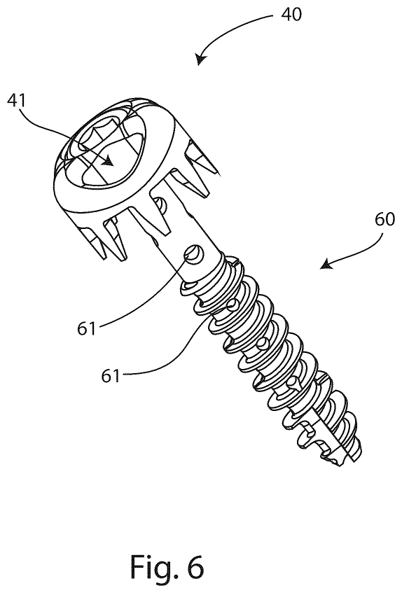

FIG. 6 is a perspective view of a cap and fastener assembly according to an embodiment of the present disclosure.

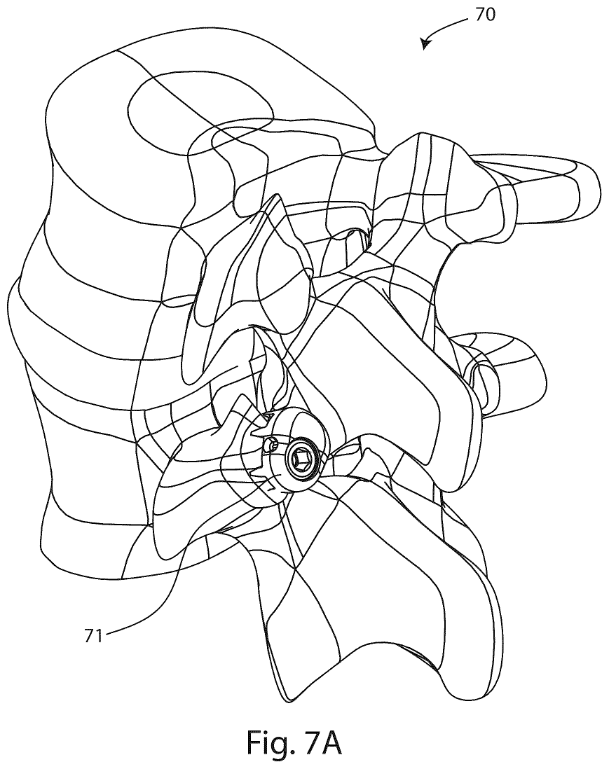

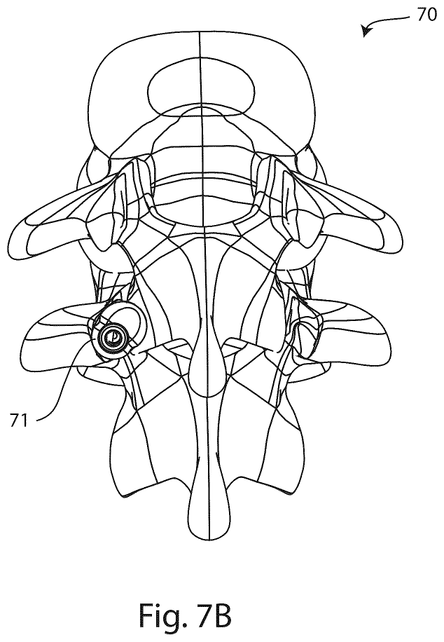

FIG. 7A is a perspective view of a lumbar portion of a spine with a facet fixation device fastened to a facet joint according to an embodiment of the present disclosure.

FIG. 7B is a posterior perspective view of the lumbar portion of the spine and the facet fixation device of FIG. 7A.

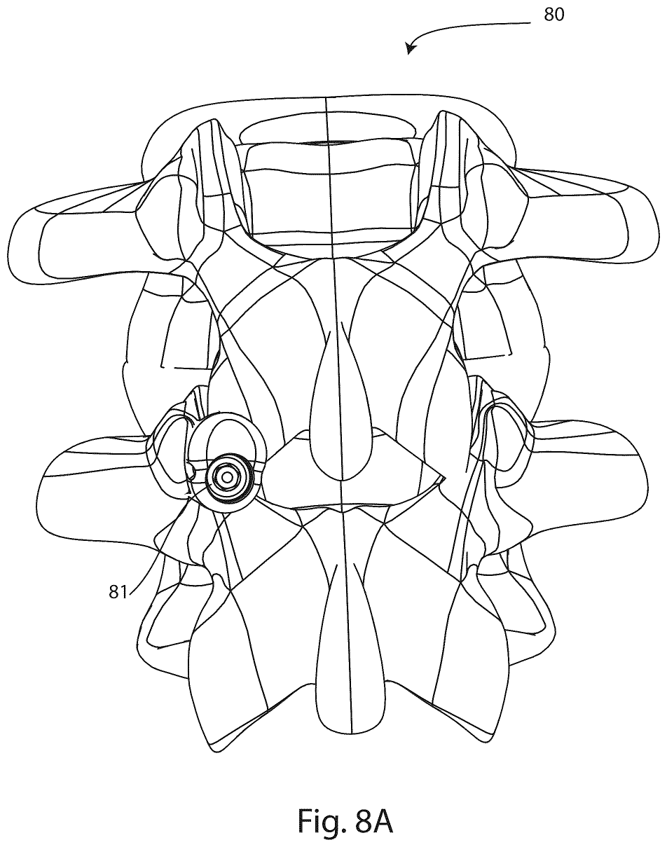

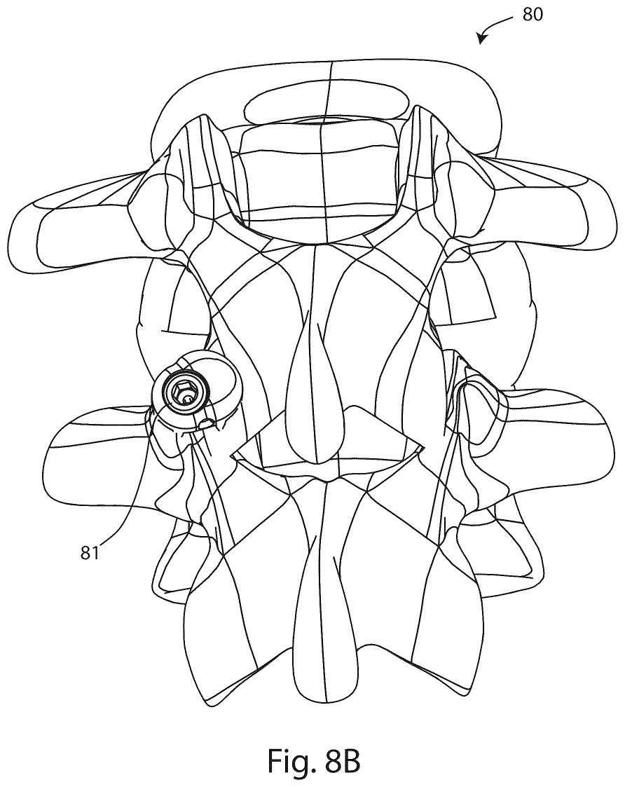

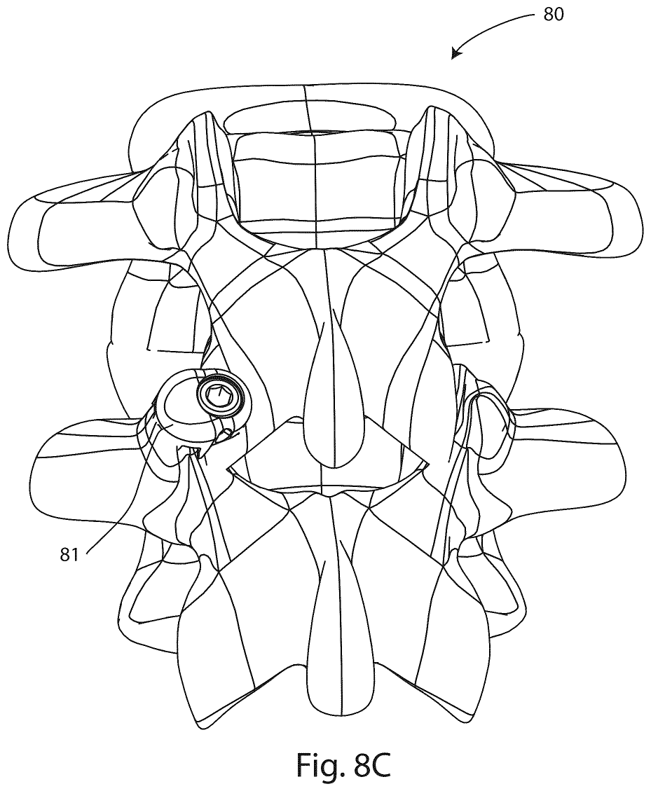

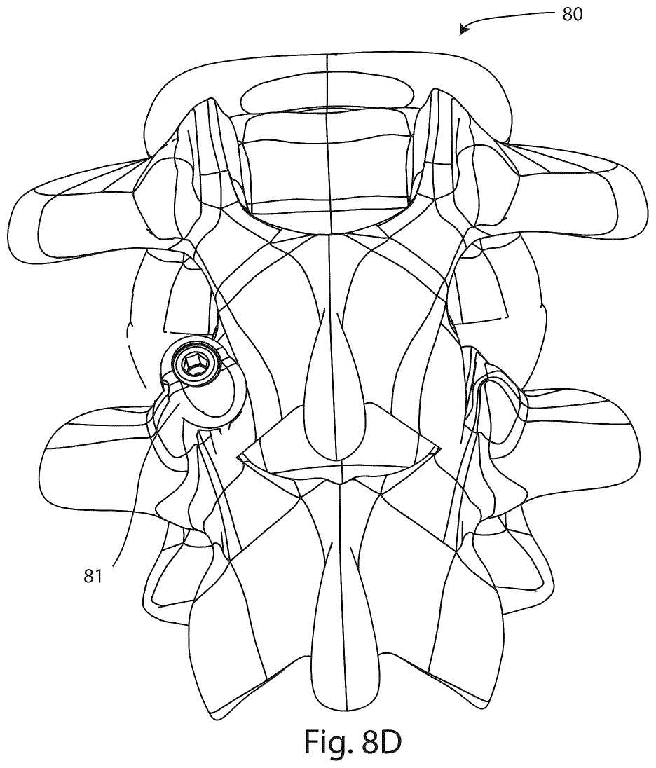

FIG. 8A is a perspective view of a lumbar portion of a spine with a facet fixation device fastened to a facet joint in a first orientation according to an embodiment of the present disclosure.

FIG. 8B is a perspective view of a lumbar portion of a spine with the facet fixation device of FIG. 8A fastened to a facet joint in a second orientation.

FIG. 8C is a perspective view of a lumbar portion of a spine with the facet fixation device of FIG. 8A fastened to a facet joint in a third orientation.

FIG. 8D is a perspective view of a lumbar portion of a spine with the facet fixation device of FIG. 8A fastened to a facet joint in a fourth orientation.

FIG. 8E is a perspective view of a lumbar portion of a spine with the facet fixation device of FIG. 8A fastened to a facet joint in a fifth orientation.

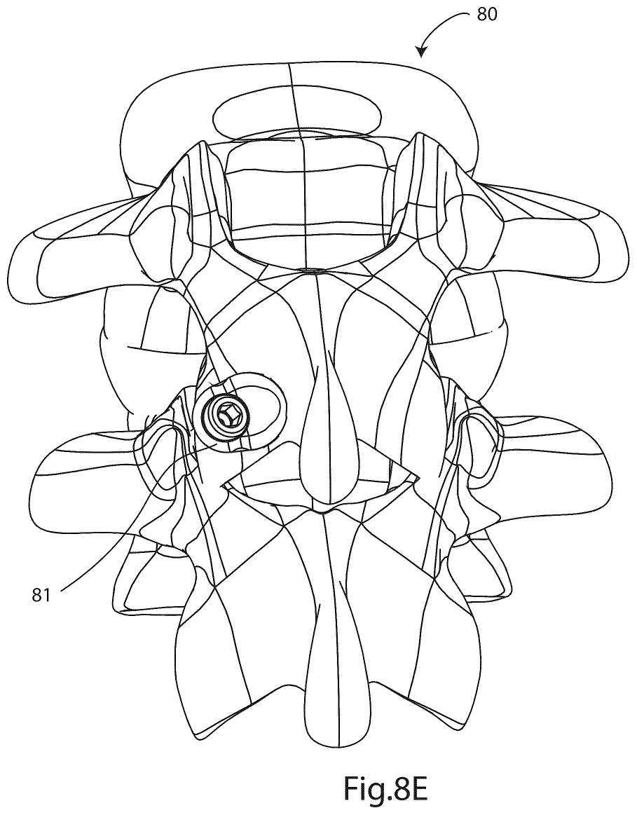

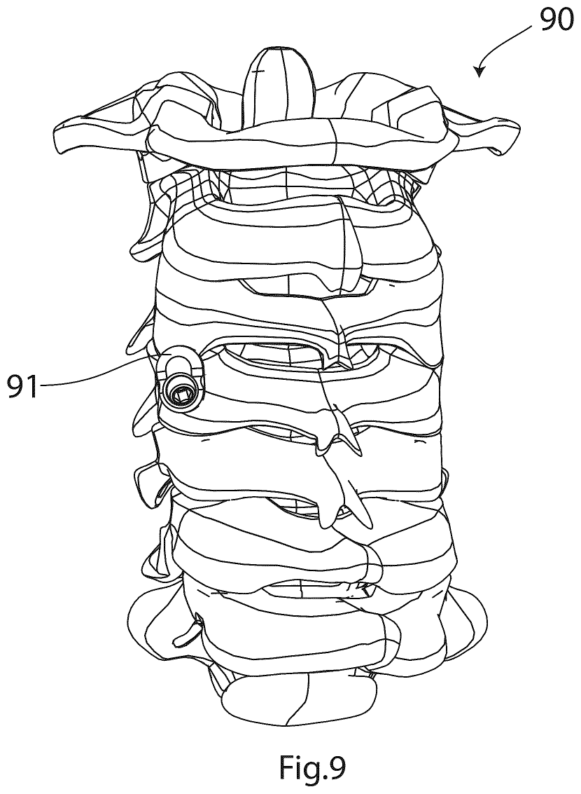

FIG. 9 is a posterior perspective view of a cervical portion of a spine with a facet fixation device fastened to a facet joint according to an embodiment of the present disclosure.

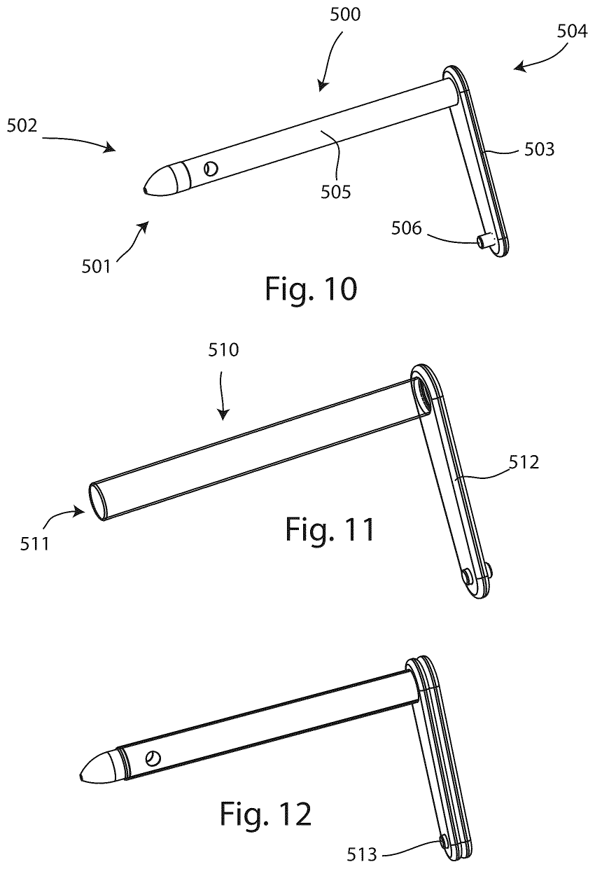

FIG. 10 is a perspective view of a dilator in accordance with an embodiment of the present disclosure.

FIG. 11 is a perspective view of a cannula in accordance with an embodiment of the present disclosure.

FIG. 12 is a perspective view of the dilator of FIG. 10 inserted into the cannula of FIG. 11.

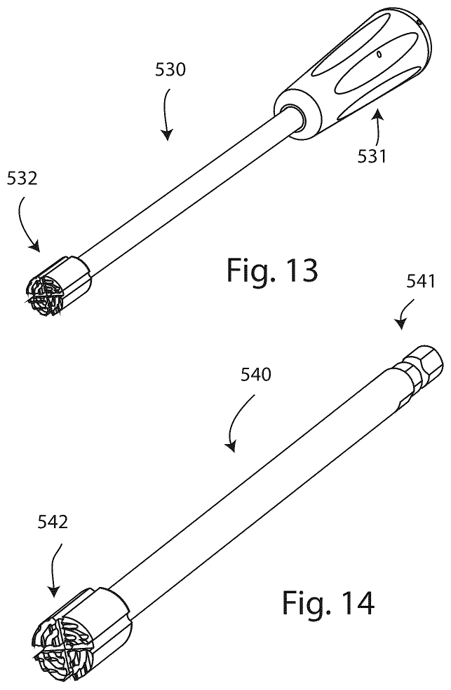

FIG. 13 is a perspective view of a manual reamer in accordance with an embodiment of the present disclosure.

FIG. 14 is a perspective view of a powered reamer in accordance with an embodiment of the present disclosure.

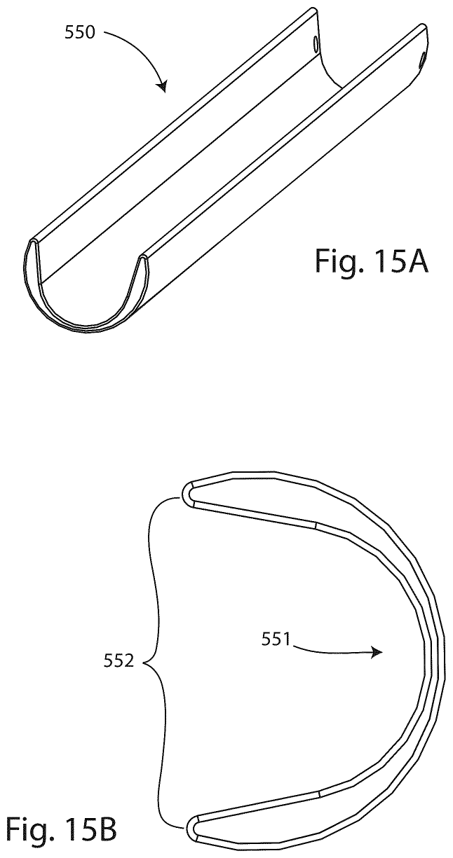

FIG. 15A is a perspective view of a guide in accordance with an embodiment of the present disclosure.

FIG. 15B is a front view of the guide of FIG. 15A.

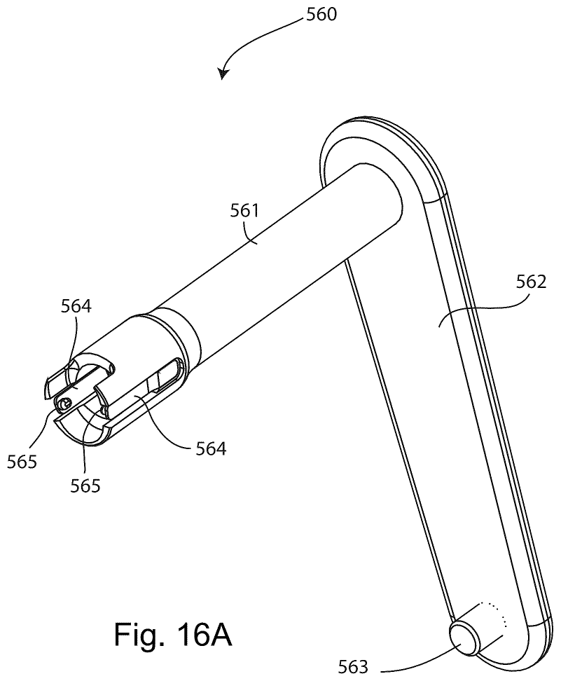

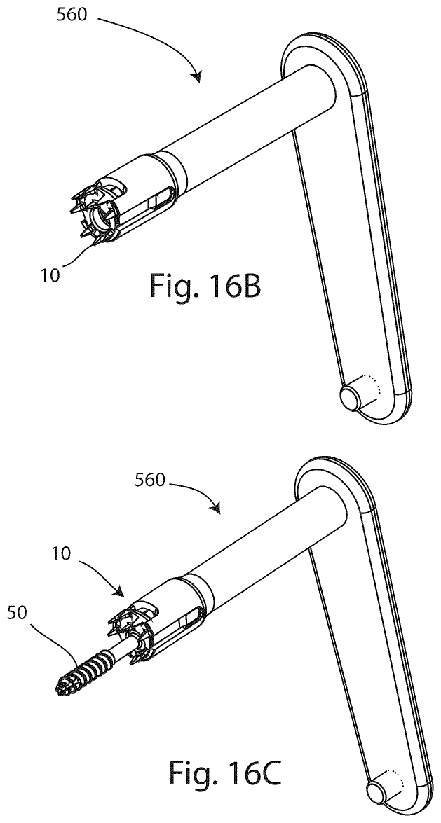

FIG. 16A is a perspective view of a guide in accordance with an embodiment of the present disclosure.

FIG. 16B is a perspective view of the guide of FIG. 16A with a cap inserted into the guide according to an embodiment of the present disclosure.

FIG. 16C is a perspective view of the guide of FIG. 16A with a cap and a fastener inserted into the guide according to an embodiment of the present disclosure.

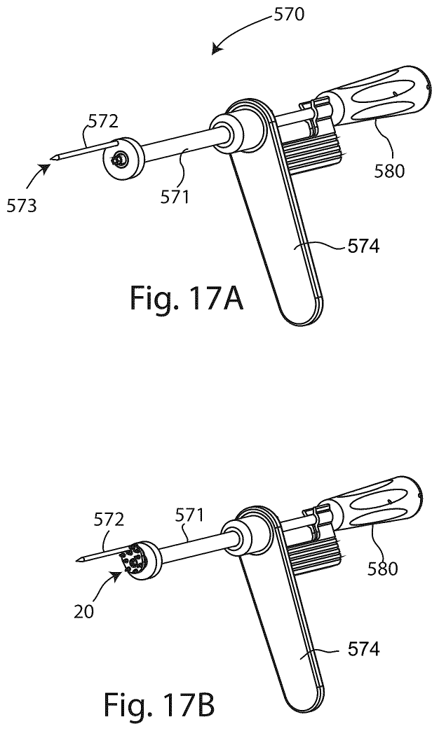

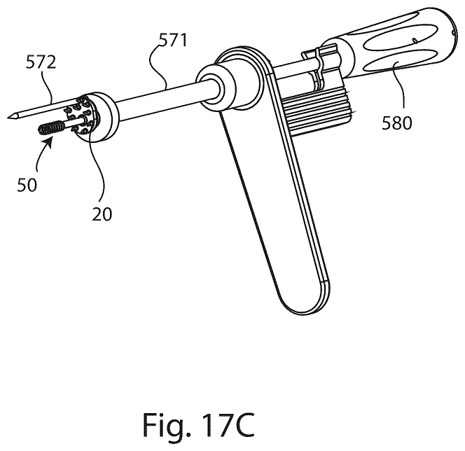

FIG. 17A is a perspective view of a guide in accordance with an embodiment of the present disclosure.

FIG. 17B is a perspective view of the guide of FIG. 17A with a cap inserted into the guide according to an embodiment of the present disclosure.

FIG. 17C is a perspective view of the guide of FIG. 17A with a cap and a fastener inserted into the guide according to an embodiment of the present disclosure.

FIG. 18 is a perspective view of a fastener driver in accordance with an embodiment of the present disclosure.

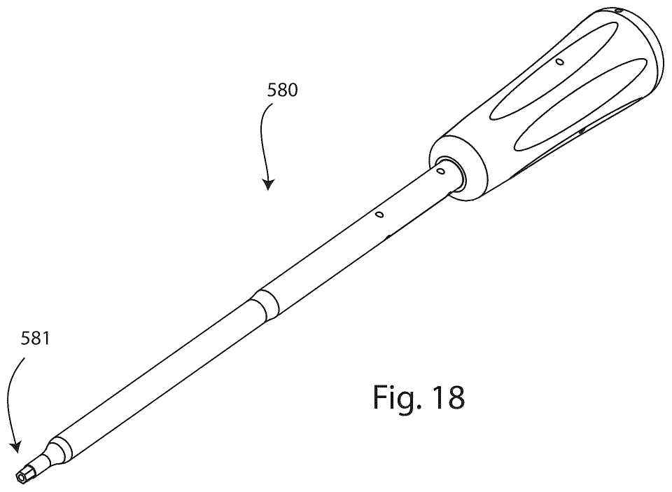

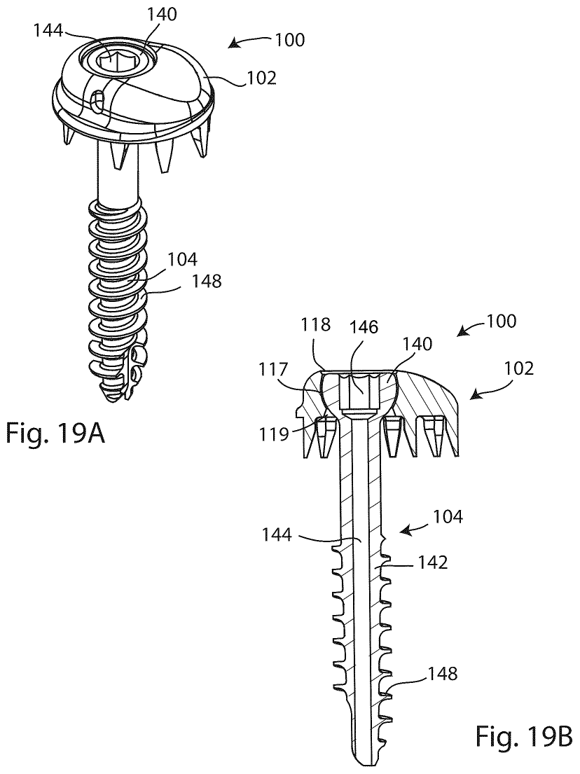

FIG. 19A is a perspective view of a facet fixation device having a cap with a circular footprint and a fastener according to an embodiment of the present disclosure.

FIG. 19B is a side cross-sectional view of the facet fixation device of FIG. 19A.

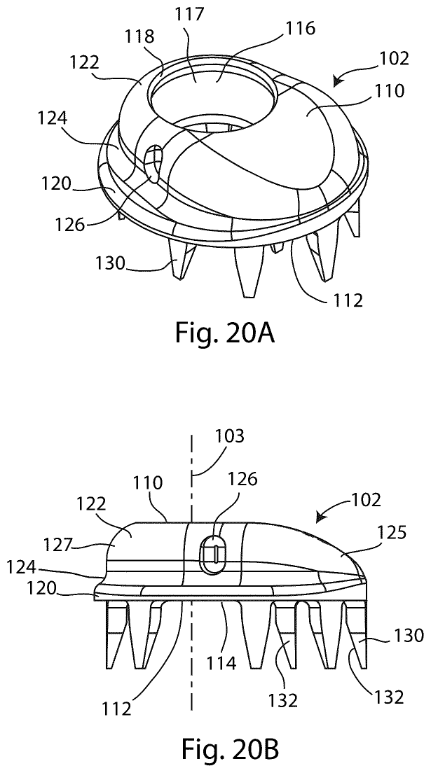

FIG. 20A is a perspective view of the cap of FIG. 19A according to an embodiment of the present disclosure.

FIG. 20B is a side view of the cap of FIG. 20A.

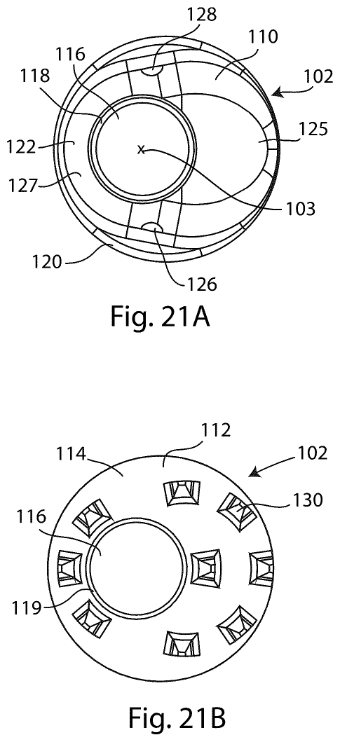

FIG. 21A is a top view of the cap of FIG. 20A.

FIG. 21B is a bottom view of the cap of FIG. 20A.

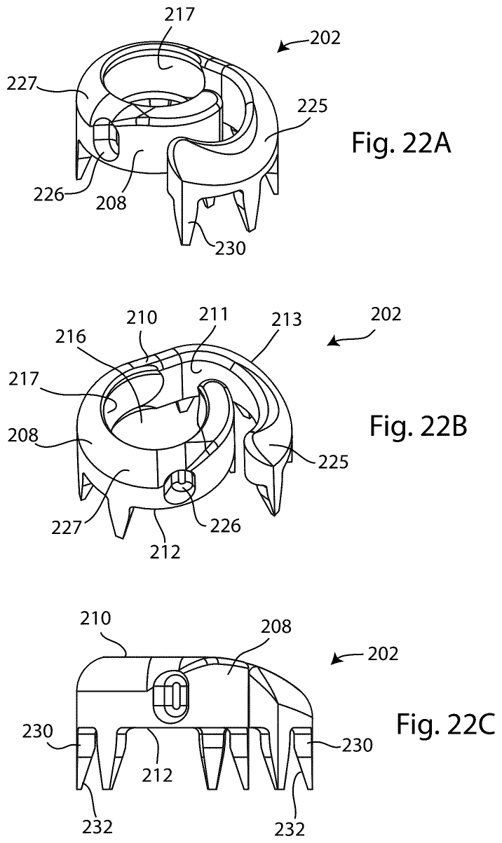

FIG. 22A is a perspective view of cap having a spiral footprint according to an embodiment of the present disclosure.

FIG. 22B is a side perspective view of the cap of FIG. 22A.

FIG. 22C is a side view of the cap of FIG. 22A.

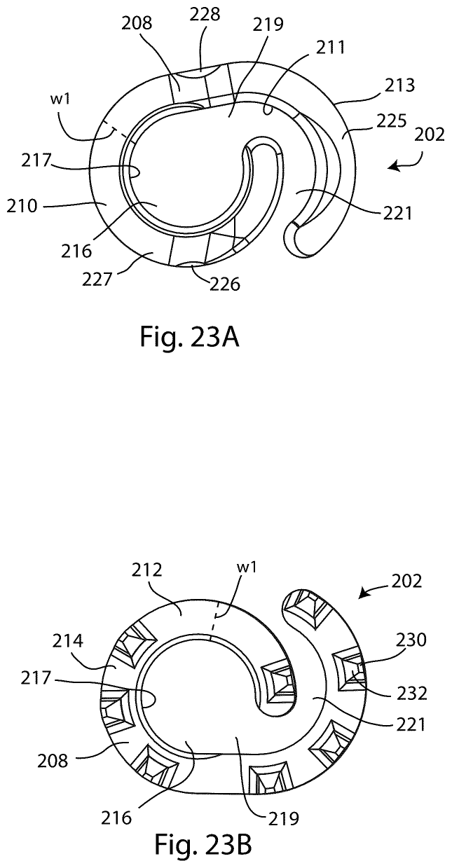

FIG. 23A is a top view of the cap of FIG. 22A.

FIG. 23B is a bottom view of the cap of FIG. 22A.

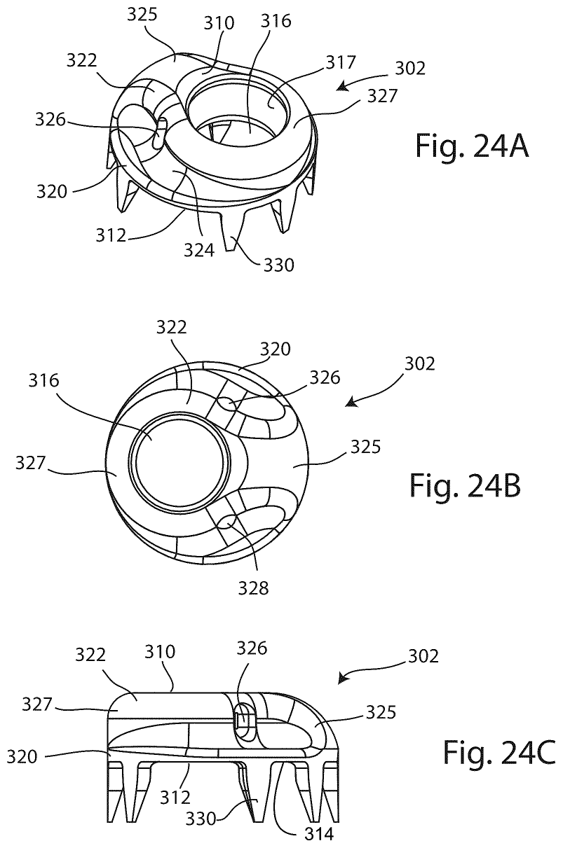

FIG. 24A is a perspective view of a cap having a circular footprint according to an embodiment of the present disclosure.

FIG. 24B is top view of the cap of FIG. 24A.

FIG. 24C is a side view of the cap of FIG. 24A.

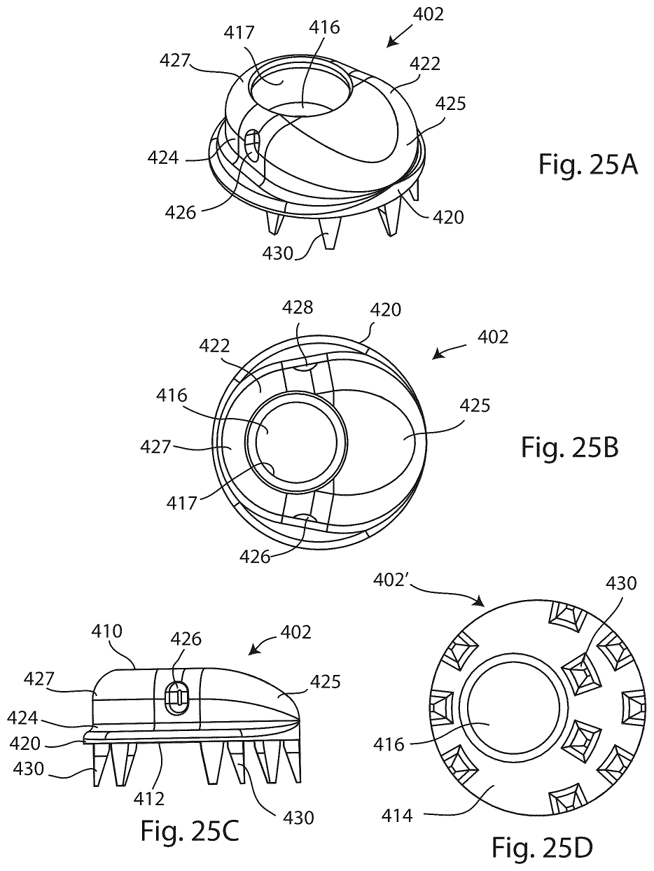

FIG. 25A is a perspective view of a cap having a circular footprint according to an embodiment of the present disclosure.

FIG. 25B is a top view of the cap of FIG. 25A.

FIG. 25C is a side view of the cap of FIG. 25A.

FIG. 25D is a bottom view of the cap of FIG. 25A.

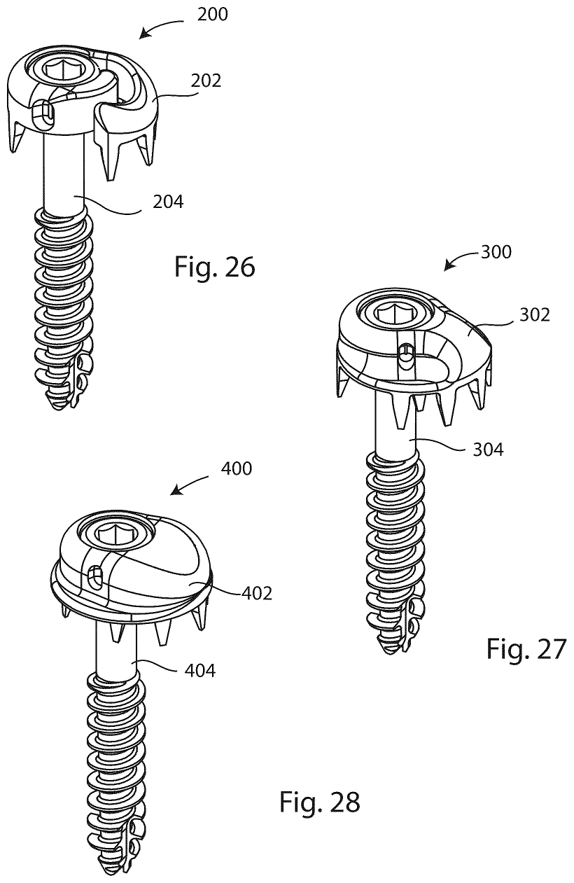

FIG. 26 is a perspective view of a facet fixation device including the cap of FIG. 22A and a fastener according to an embodiment of the present disclosure.

FIG. 27 is a perspective view of a facet fixation device including the cap of FIG. 24A and a fastener according to an embodiment of the present disclosure.

FIG. 28 is a perspective view of a facet fixation device including the cap of FIG. 25A and a fastener according to an embodiment of the present disclosure.

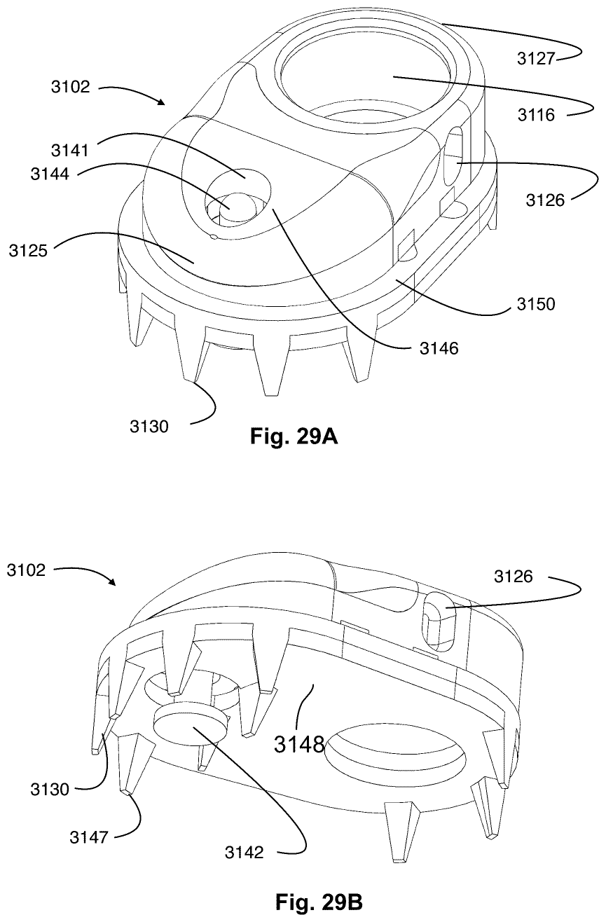

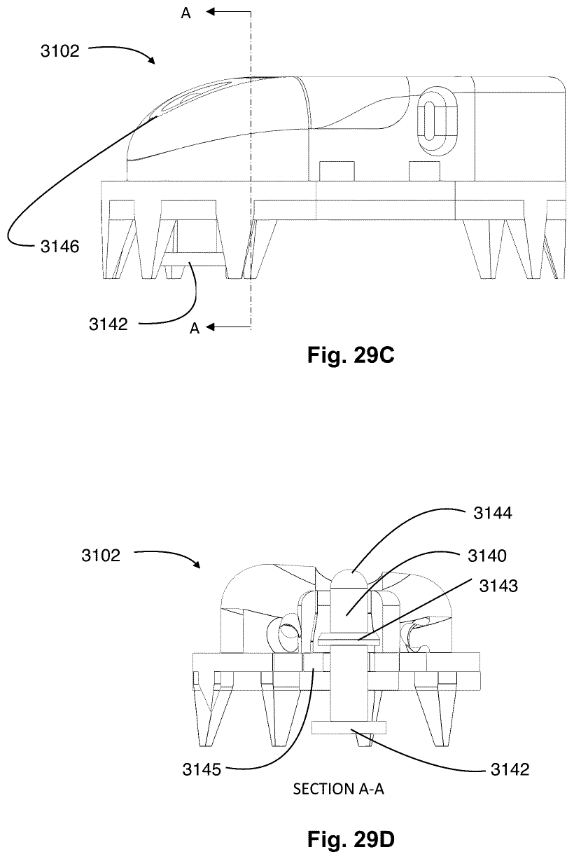

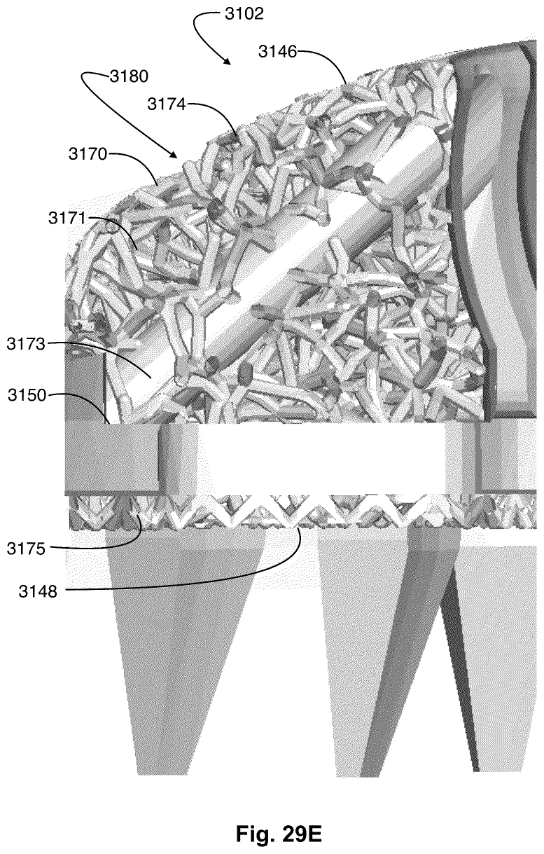

FIG. 29A is a top perspective view of a cap in accordance with an embodiment of the present disclosure.

FIG. 29B is a bottom perspective view of the cap of FIG. 29A.

FIG. 29C is a side view of the cap of FIG. 29A.

FIG. 29D is a front section view of the cap of FIG. 29C.

FIG. 29E is a partial section view of the cap of FIG. 29A in accordance with an embodiment of the present disclosure.

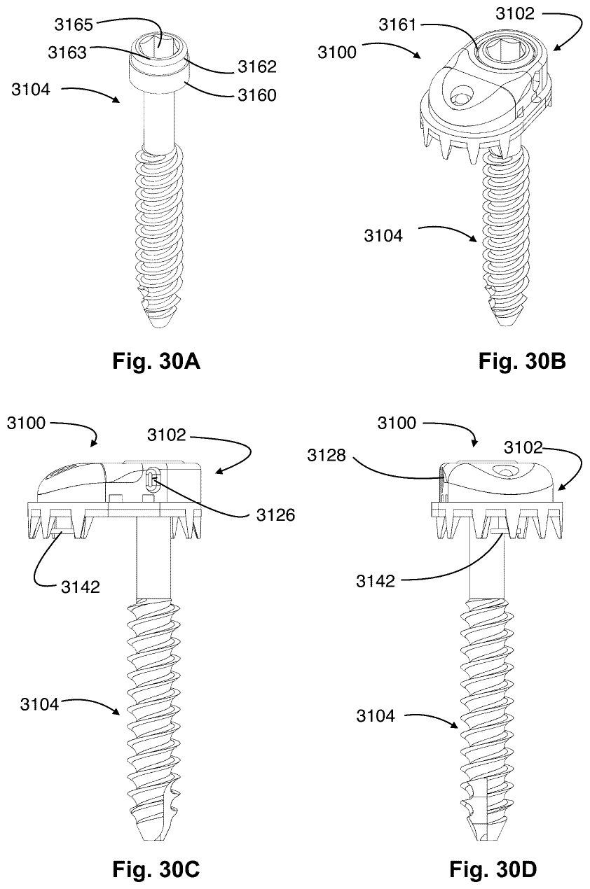

FIG. 30A is a perspective view of a fastener according to an embodiment of the present disclosure.

FIG. 30B is a perspective view of a facet fixation device in accordance with an embodiment of the present disclosure, including the cap of FIG. 29A and the fastener of FIG. 30A.

FIG. 30C is a side view of the facet fixation device of FIG. 30B.

FIG. 30D is a front view of the facet fixation device of FIG. 30B.

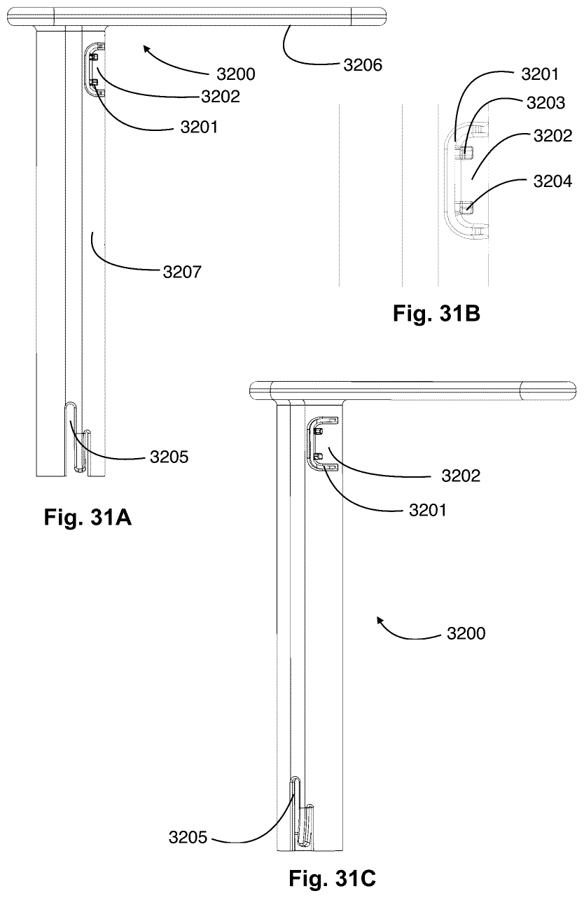

FIG. 31A is a side view of an inserter tube according to an embodiment of the present disclosure.

FIG. 31B is a partial detail view of the inserter tube of FIG. 31A.

FIG. 31C is an alternate side view of the inserter tube of FIG. 31A.

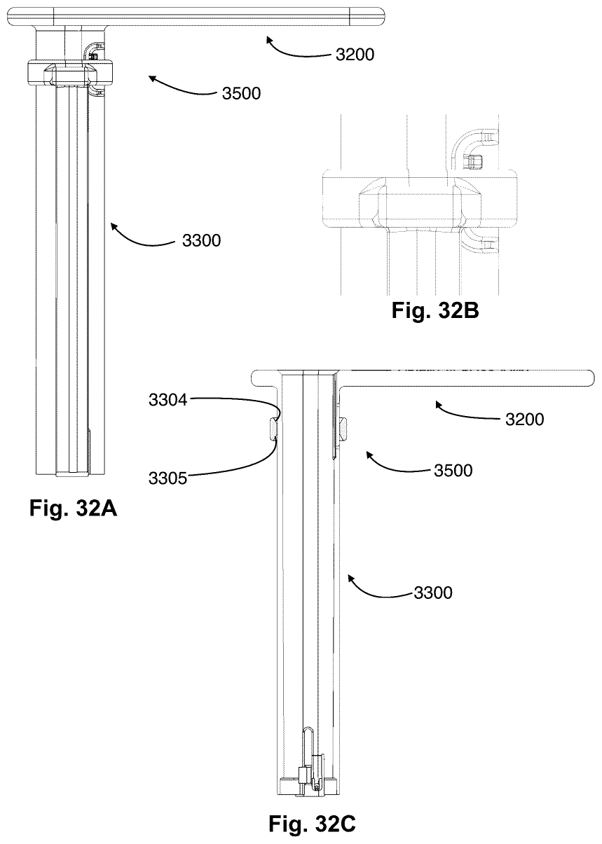

FIG. 32A is a side view of an inserter assembly according to an embodiment of the present disclosure.

FIG. 32B is a partial detail view of the inserter assembly of FIG. 32A.

FIG. 32C is an alternate side view of the inserter assembly of FIG. 32A.

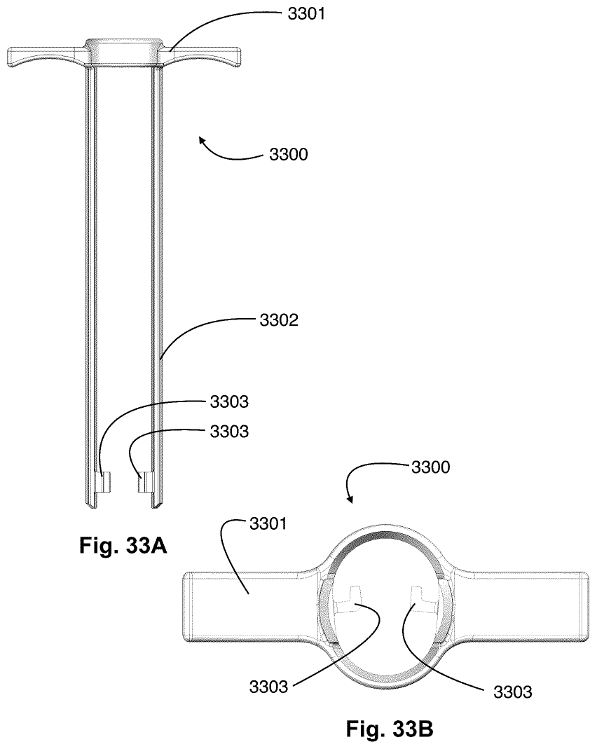

FIG. 33A is a front view of an inserter lockout according to an embodiment of the present disclosure.

FIG. 33B is a top view of the inserter lockout of FIG. 33A.

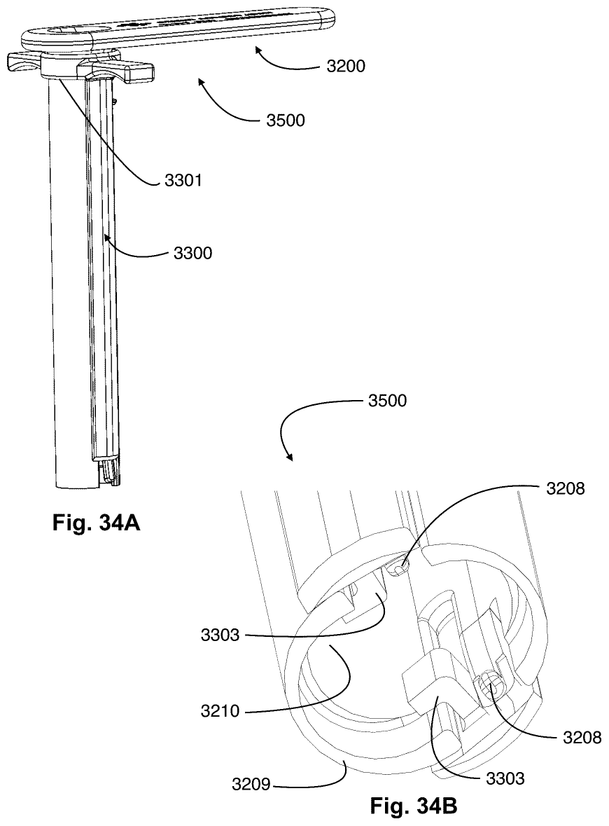

FIG. 34A is a perspective view of the inserter assembly of FIG. 32A.

FIG. 34B is a perspective view of the inserter assembly of FIG. 32A.

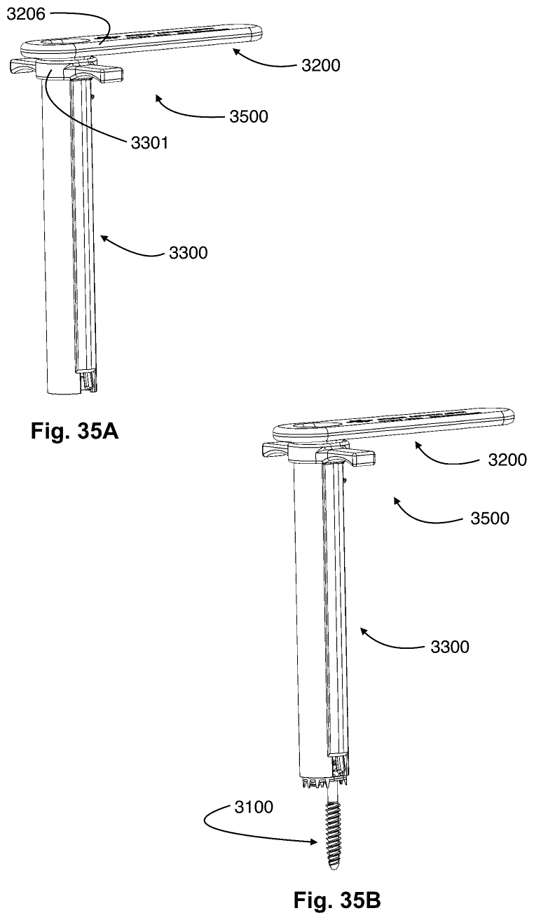

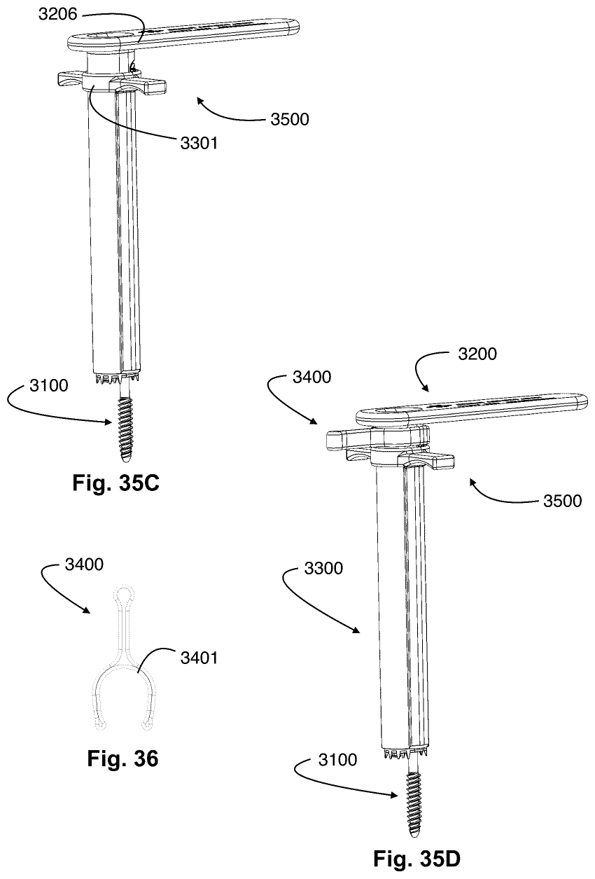

FIG. 35A is a perspective view of the inserter assembly of FIG. 32A.

FIG. 35B is a perspective view of the inserter assembly of FIG. 35A including a facet fixation device according to an embodiment of the present disclosure.

FIG. 35C is a perspective view of the inserter assembly of FIG. 35A including a facet fixation device according to an embodiment of the present disclosure.

FIG. 35D is a perspective view of the inserter assembly of FIG. 35A including a facet fixation device and the removeable lock of FIG. 36 according to an embodiment of the present disclosure.

FIG. 36 is a top view of a removeable lock according to an embodiment of the present disclosure.

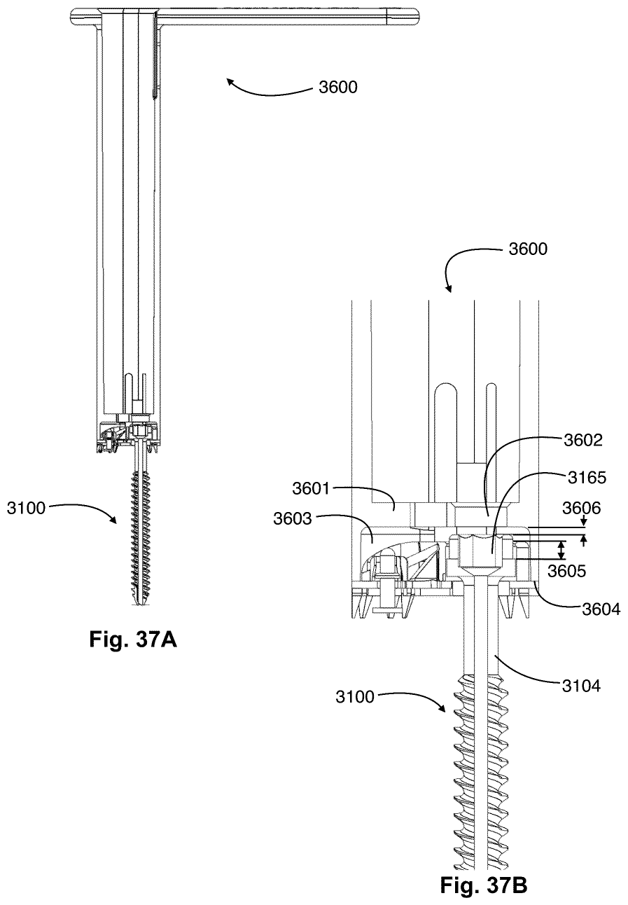

FIG. 37A is a side view of an inserter assembly according to an embodiment of the present disclosure.

FIG. 37B is a partial detail side view of the inserter assembly of FIG. 37A.

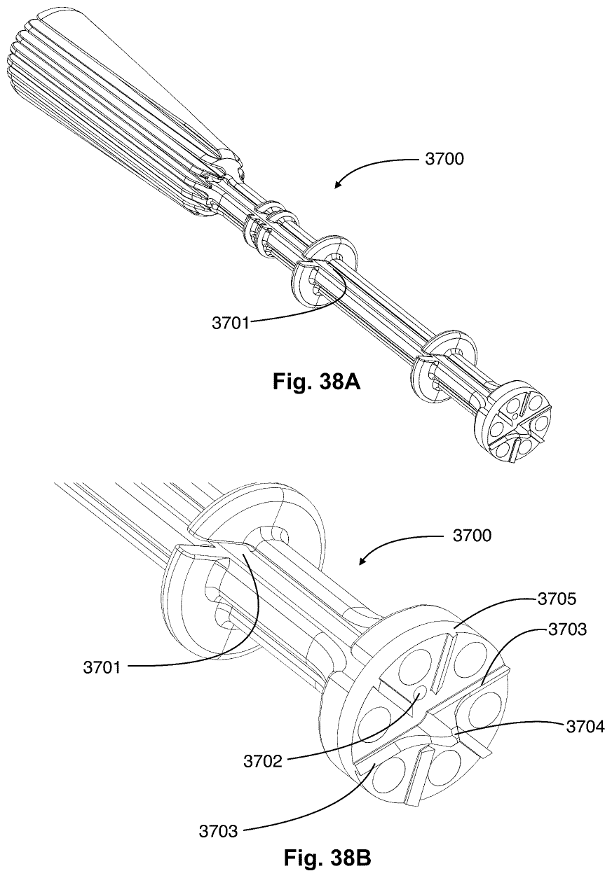

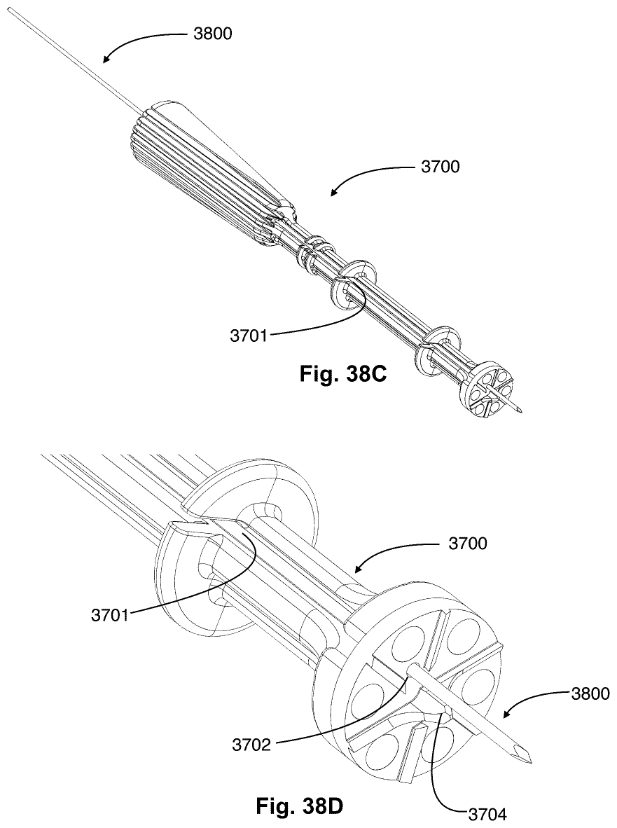

FIG. 38A is a perspective view of a reamer according to an embodiment of the present disclosure.

FIG. 38B is a partial detail perspective view of the reamer of FIG. 38A.

FIG. 38C is a perspective view of a reamer and k-wire assembly according to an embodiment of the present disclosure.

FIG. 38D is a partial detail perspective view of the reamer and k-wire assembly of FIG. 38C.

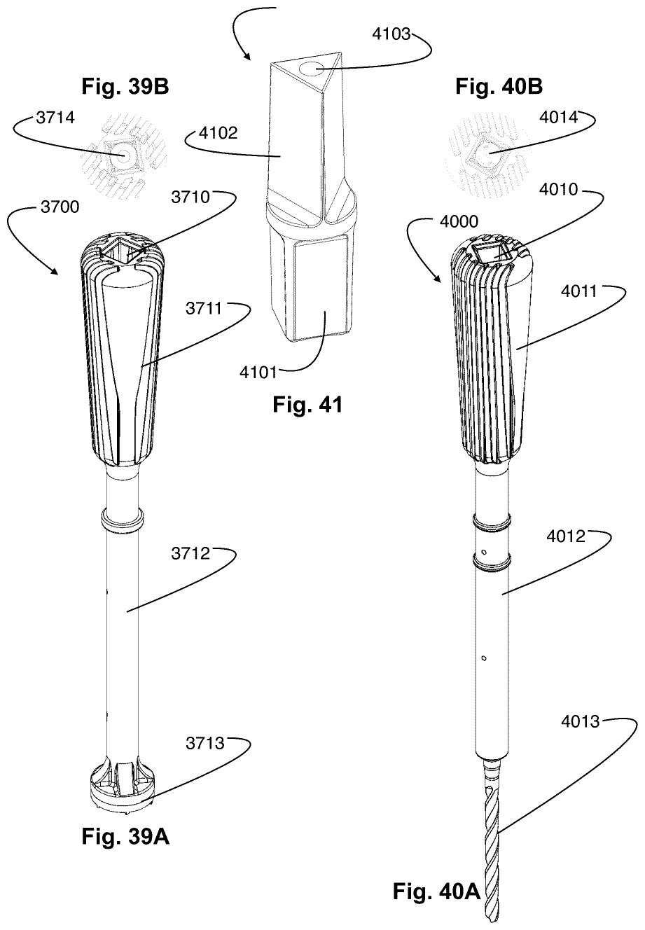

FIG. 39A is a perspective view of a reamer according to an embodiment of the present disclosure.

FIG. 39B is a top view of the reamer of FIG. 39A.

FIG. 40A is a perspective view of a drill according to an embodiment of the present disclosure.

FIG. 40B is a top view of the drill of FIG. 40A.

FIG. 41 is a perspective view of a power adapter according to an embodiment of the present disclosure.

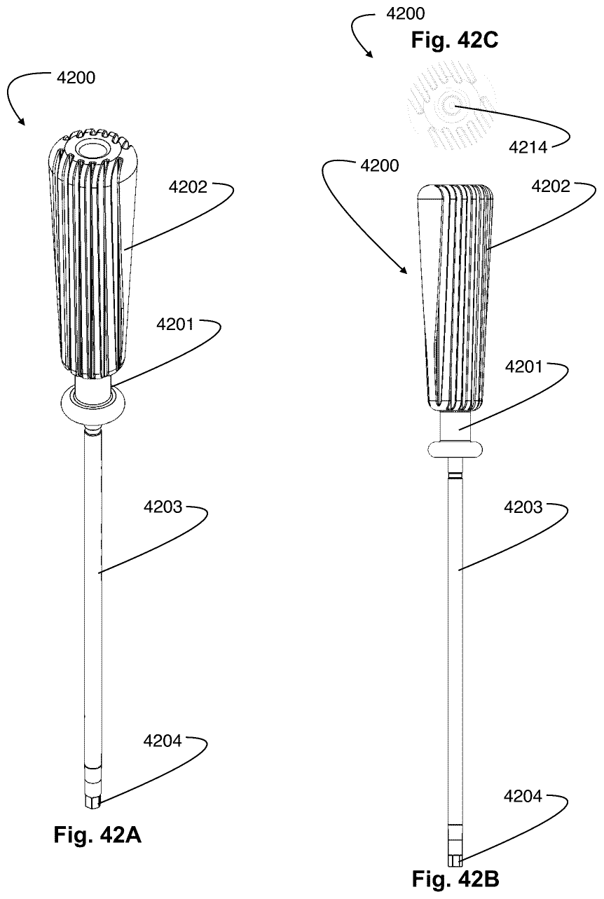

FIG. 42A is a perspective view of a driver according to an embodiment of the present disclosure.

FIG. 42B is a front view of the driver of FIG. 42A.

FIG. 42C is a top view of the driver of FIG. 42A.

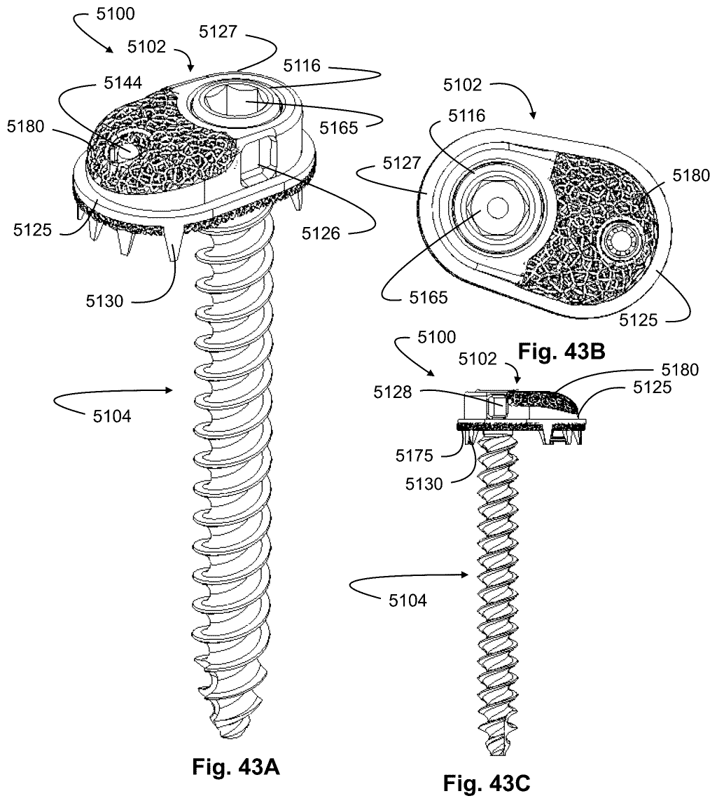

FIG. 43A is a perspective view of a facet fixation device according to an embodiment of the present disclosure.

FIG. 43B is a top view of the facet fixation device of FIG. 43A.

FIG. 43C is a side view of the facet fixation device of FIG. 43A.

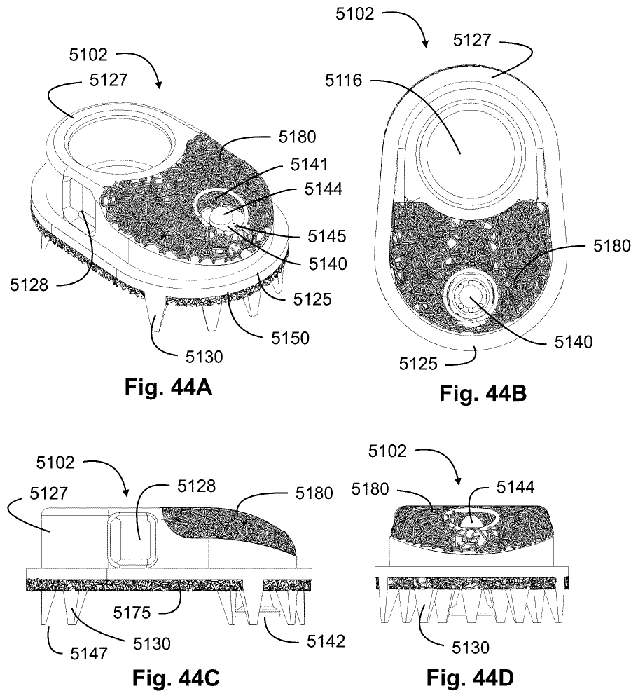

FIG. 44A is a perspective view of a cap according to an embodiment of the present disclosure.

FIG. 44B is a top view of the cap of FIG. 44A.

FIG. 44C is a side view of the cap of FIG. 44A.

FIG. 44D is a front view of the cap of FIG. 44A.

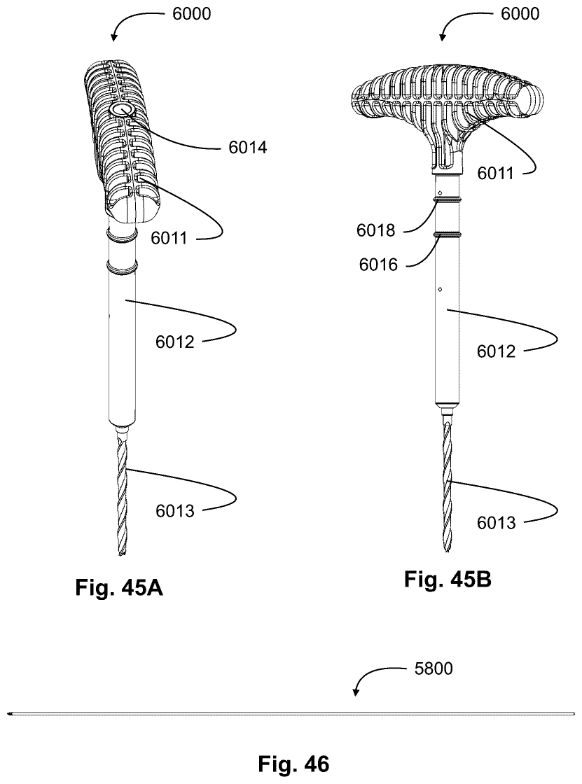

FIG. 45A is a perspective view of a drill according to an embodiment of the present disclosure.

FIG. 45B is a front view of the drill of FIG. 45A.

FIG. 46 is a side view of a k-wire according to an embodiment of the present disclosure.

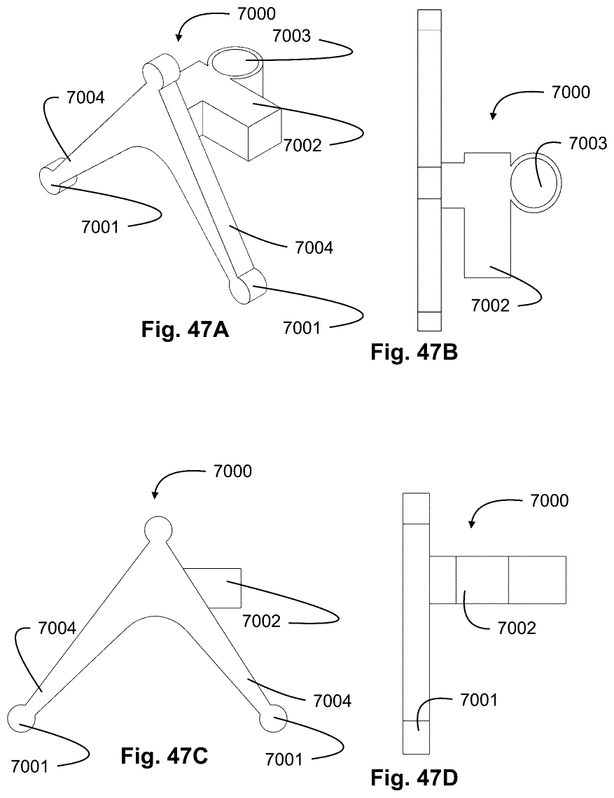

FIG. 47A is a perspective view of an array according to an embodiment of the present disclosure.

FIG. 47B is a top view of the array of FIG. 47A.

FIG. 47C is a front view of the array of FIG. 47A.

FIG. 47D is a side view of the array of FIG. 47A.

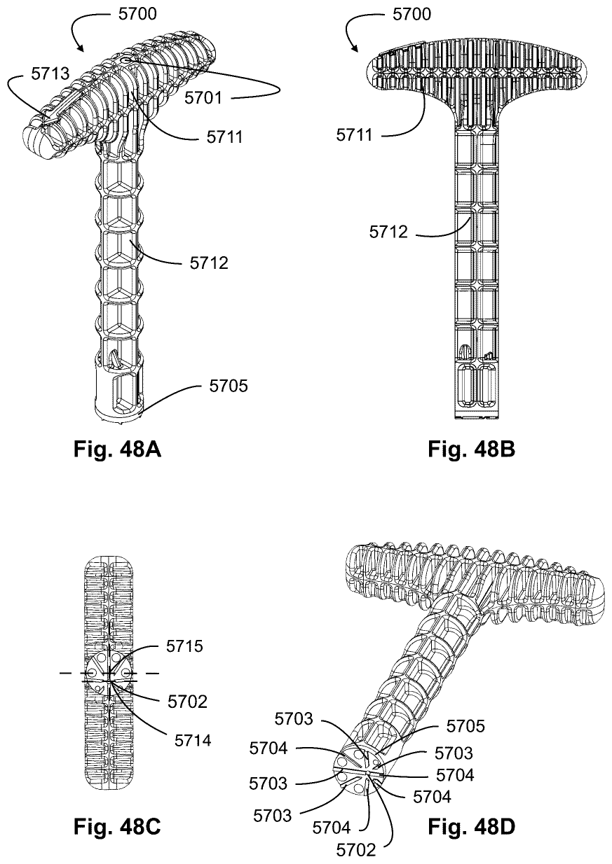

FIG. 48A is a perspective view of a reamer according to an embodiment of the present disclosure.

FIG. 48B is a front view of the reamer of FIG. 48A.

FIG. 48C is a bottom view of the reamer of FIG. 48A.

FIG. 48D is a perspective view of the reamer of FIG. 48A.

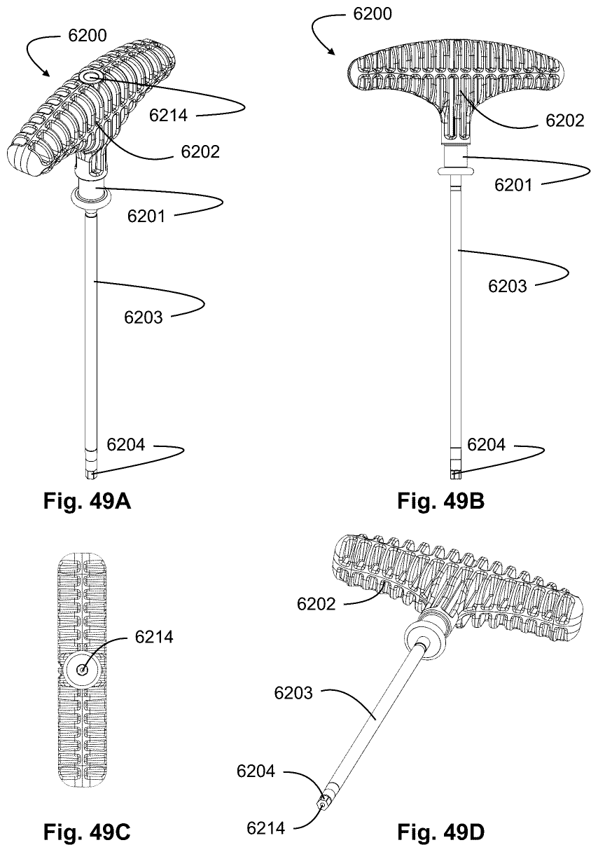

FIG. 49A is a perspective view of a driver according to an embodiment of the present disclosure.

FIG. 49B is a front view of the driver of FIG. 49A.

FIG. 49C is a bottom view of the driver of FIG. 49A.

FIG. 49D is a perspective view of the driver of FIG. 49A.

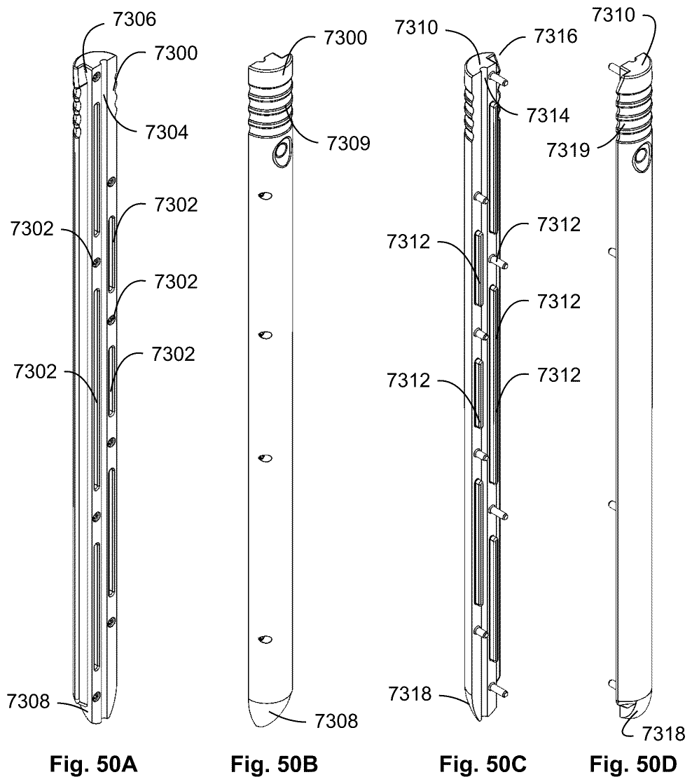

FIG. 50A is a perspective view of a first small dilator portion according to an embodiment of the present disclosure.

FIG. 50B is a perspective view of the first small dilator portion of FIG. 50A.

FIG. 50C is a perspective view of a second small dilator portion according to an embodiment of the present disclosure.

FIG. 50D is a perspective view of the second small dilator portion of FIG. 50C.

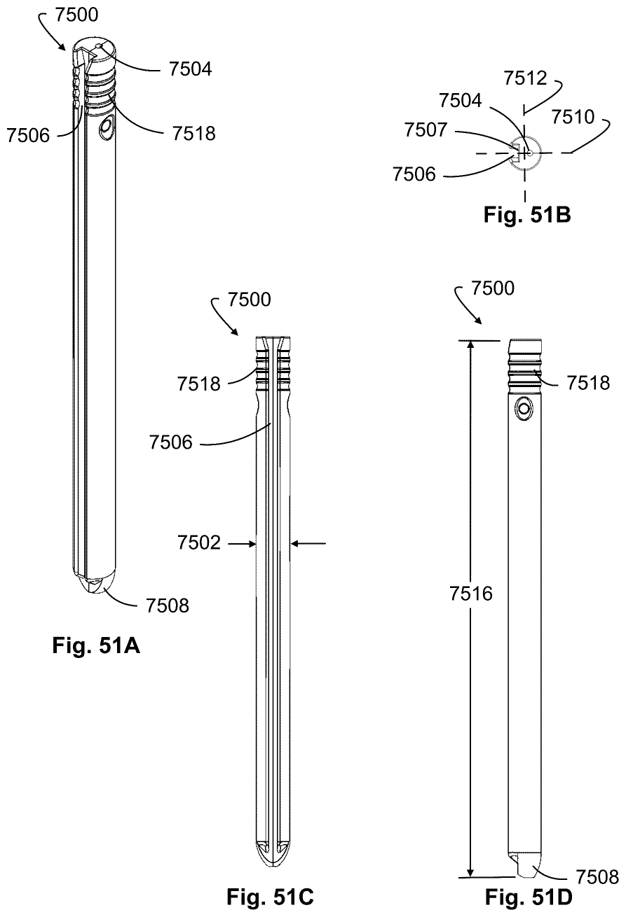

FIG. 51A is a perspective view of a small dilator according to an embodiment of the present disclosure.

FIG. 51B is a top view of the small dilator of FIG. 51A.

FIG. 51C is a front view of the small dilator of FIG. 51A.

FIG. 51D is a side view of the small dilator of FIG. 51A.

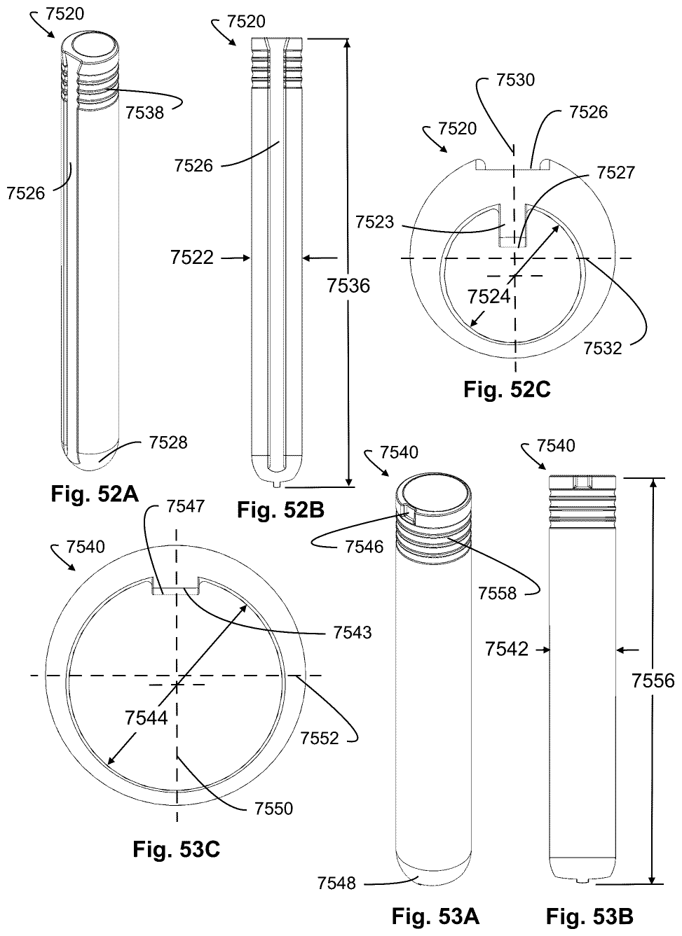

FIG. 52A is a perspective view of a medium dilator according to an embodiment of the present disclosure.

FIG. 52B is a front view of the medium dilator of FIG. 52A.

FIG. 52C is a bottom view of the medium dilator of FIG. 52A.

FIG. 53A is a perspective view of a large dilator according to an embodiment of the present disclosure.

FIG. 53B is a front view of the large dilator of FIG. 53A.

FIG. 53C is a bottom view of the large dilator of FIG. 53A.

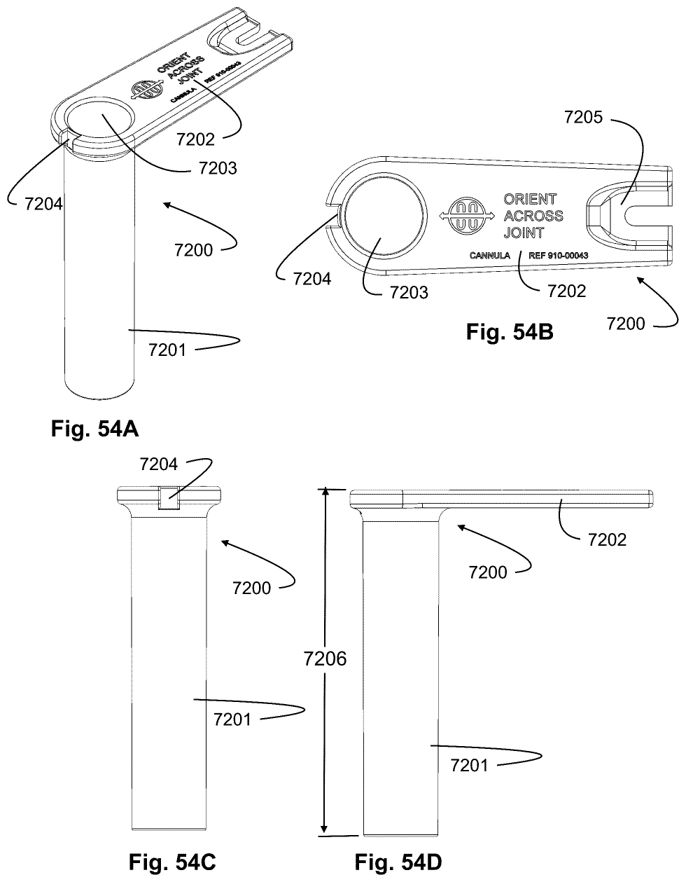

FIG. 54A is a perspective view of a cannula according to an embodiment of the present disclosure.

FIG. 54B is a top view of the cannula of FIG. 54A.

FIG. 54C is a front view of the cannula of FIG. 54A.

FIG. 54D is a side view of the cannula of FIG. 54A.

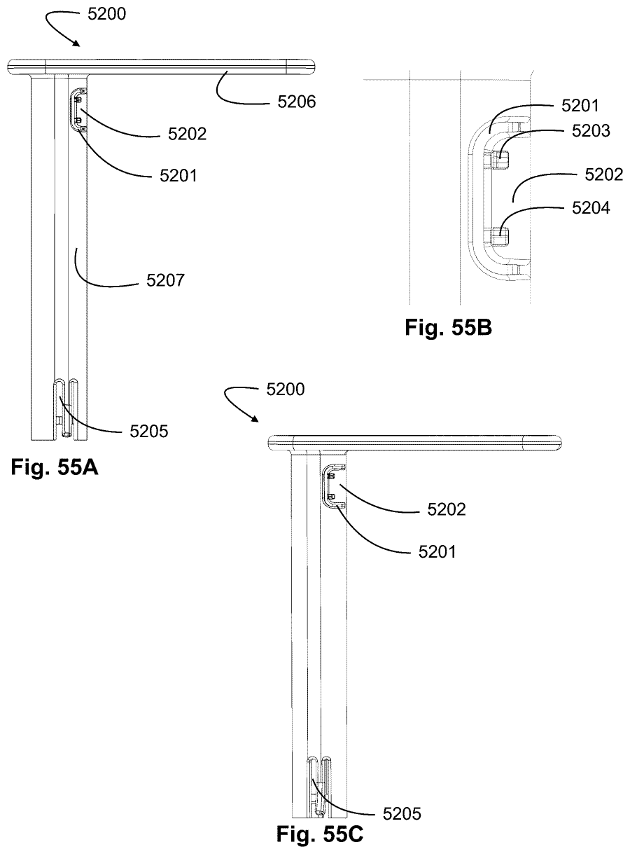

FIG. 55A is a side view of an inserter tube according to an embodiment of the present disclosure.

FIG. 55B is a partial detail view of the inserter tube of FIG. 55A.

FIG. 55C is an alternate side view of the inserter tube of FIG. 55A.

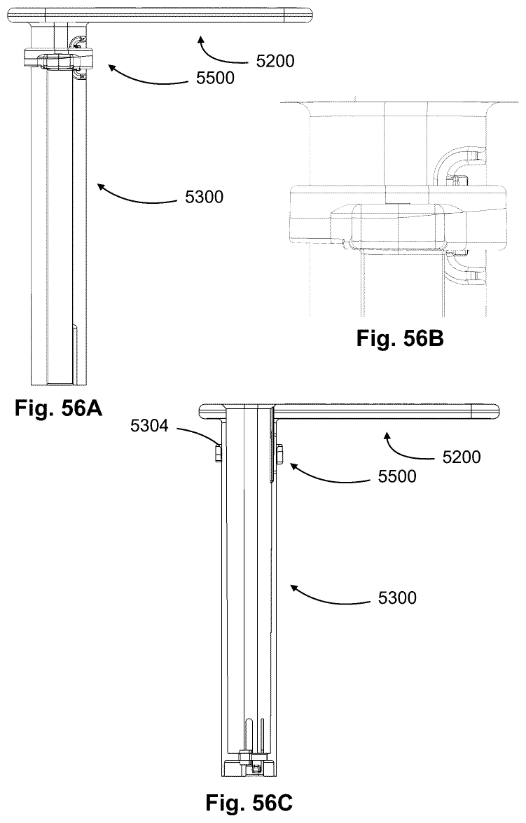

FIG. 56A is a side view of an inserter assembly according to an embodiment of the present disclosure.

FIG. 56B is a partial detail view of the inserter assembly of FIG. 56A.

FIG. 56C is an alternate side view of the inserter assembly of FIG. 56A.

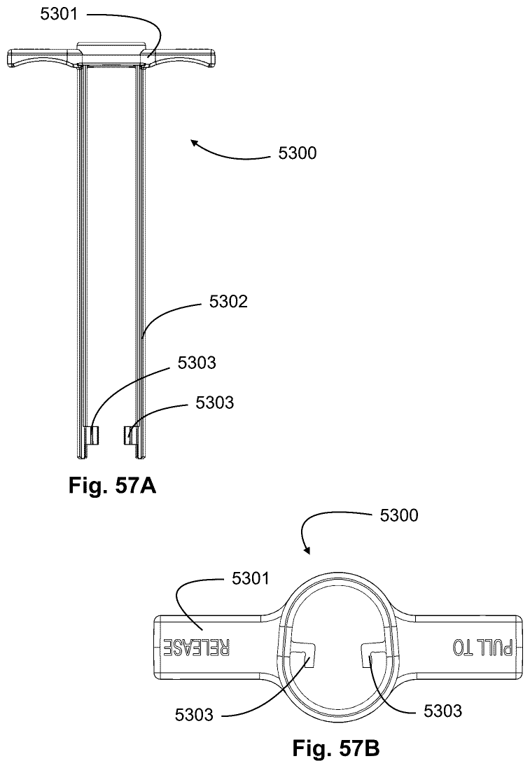

FIG. 57A is a front view of an inserter lockout according to an embodiment of the present disclosure.

FIG. 57B is a top view of the inserter lockout of FIG. 57A.

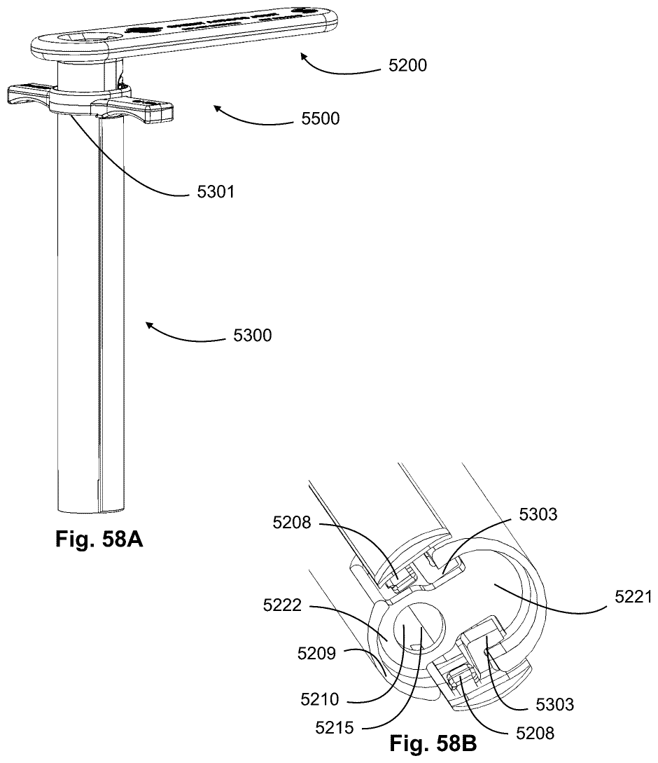

FIG. 58A is a perspective view of the inserter assembly of FIG. 56A.

FIG. 58B is a perspective view of the inserter assembly of FIG. 56A.

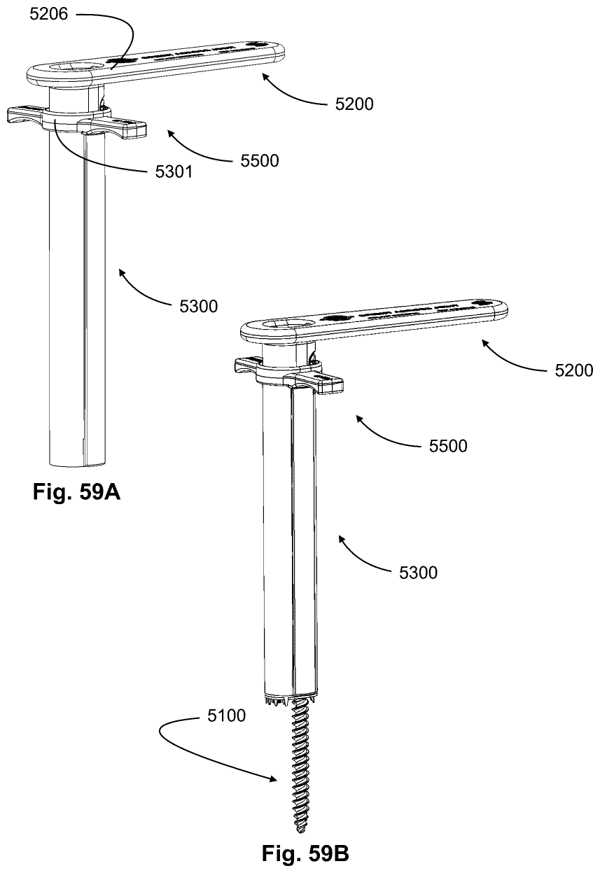

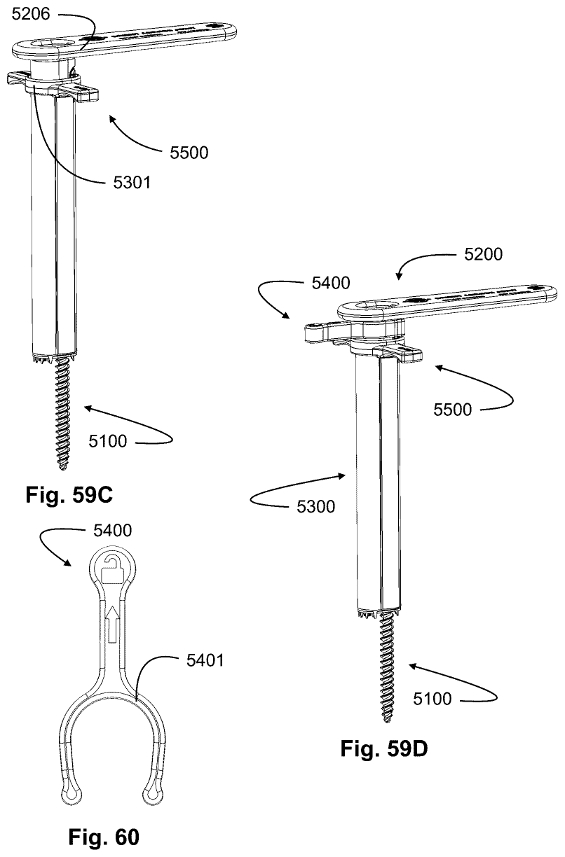

FIG. 59A is a perspective view of the inserter assembly of FIG. 56A.

FIG. 59B is a perspective view of the inserter assembly of FIG. 59A including a facet fixation device according to an embodiment of the present disclosure.

FIG. 59C is a perspective view of the inserter assembly of FIG. 59A including a facet fixation device according to an embodiment of the present disclosure.

FIG. 59D is a perspective view of the inserter assembly of FIG. 59A including a facet fixation device and the removeable lock of FIG. 60 according to an embodiment of the present disclosure.

FIG. 60 is a top view of a removeable lock according to an embodiment of the present disclosure.

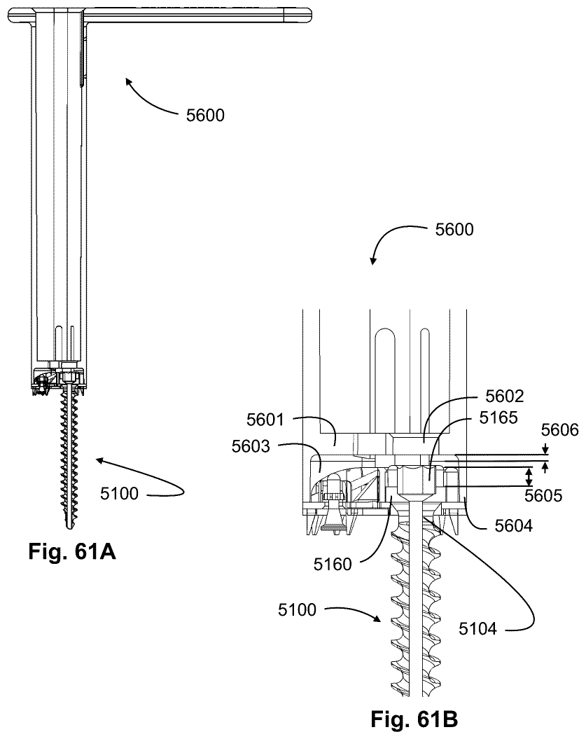

FIG. 61A is a side view of an inserter assembly according to an embodiment of the present disclosure.

FIG. 61B is a partial detail side view of the inserter assembly of FIG. 61A.

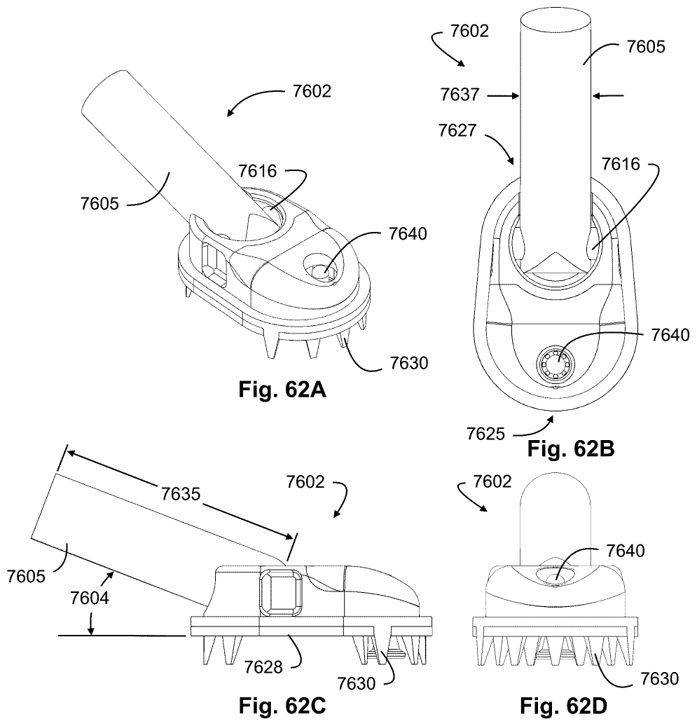

FIG. 62A is a perspective view of a cap according to an embodiment of the present disclosure.

FIG. 62B is a top view of the cap of FIG. 62A.

FIG. 62C is a side view of the cap of FIG. 62A.

FIG. 62D is a front view of the cap of FIG. 62A.

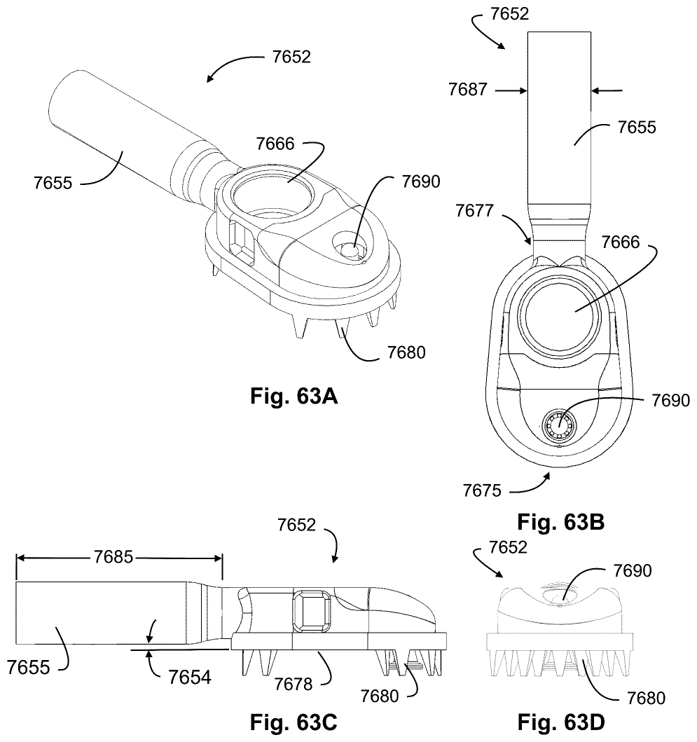

FIG. 63A is a perspective view of a cap according to an embodiment of the present disclosure.

FIG. 63B is a top view of the cap of FIG. 63A.

FIG. 63C is a side view of the cap of FIG. 63A.

FIG. 63D is a front view of the cap of FIG. 63A.

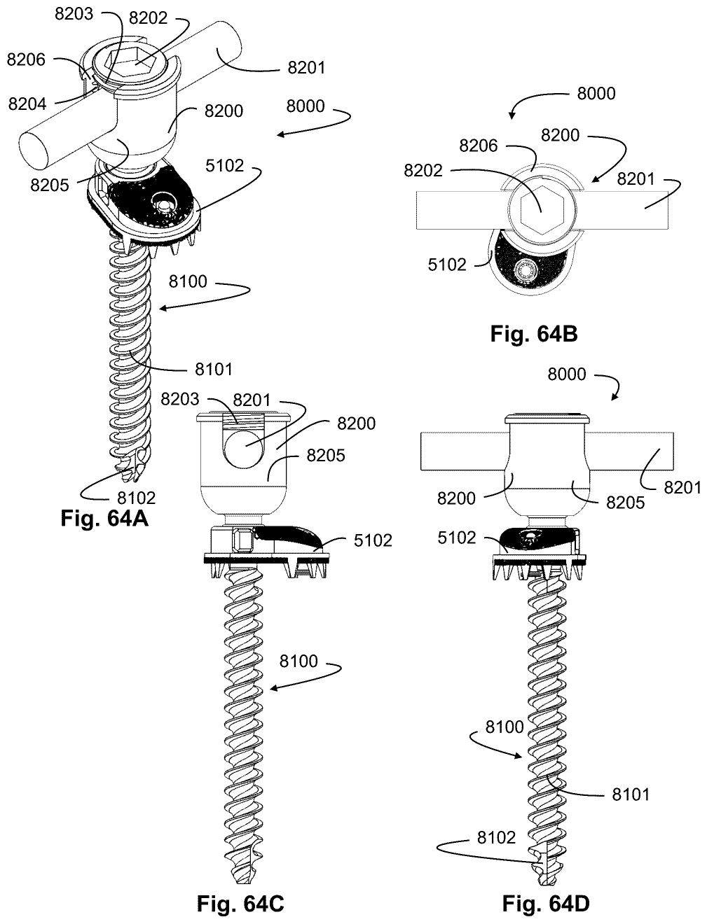

FIG. 64A is a perspective view of a facet fixation device according to an embodiment of the present disclosure.

FIG. 64B is a top view of the facet fixation device of FIG. 64A.

FIG. 64C is a side view of the facet fixation device of FIG. 64A.

FIG. 64D is a front view of the facet fixation device of FIG. 64A.

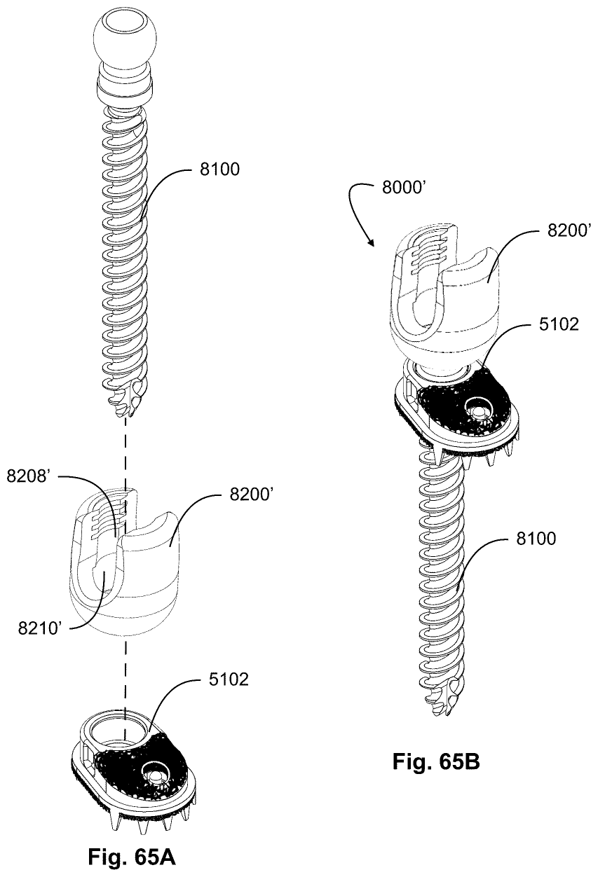

FIG. 65A is an exploded perspective view of a facet fixation device according to an embodiment of the present disclosure.

FIG. 65B is a perspective view of the facet fixation device of FIG. 65A.

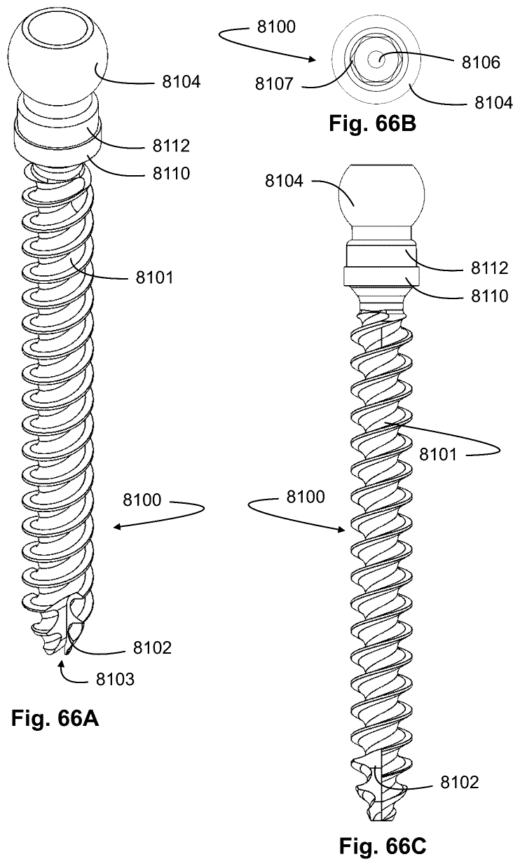

FIG. 66A is a perspective view of a fastener according to an embodiment of the present disclosure.

FIG. 66B is a top view of the fastener of FIG. 66A.

FIG. 66C is a front view of the fastener of FIG. 66A.

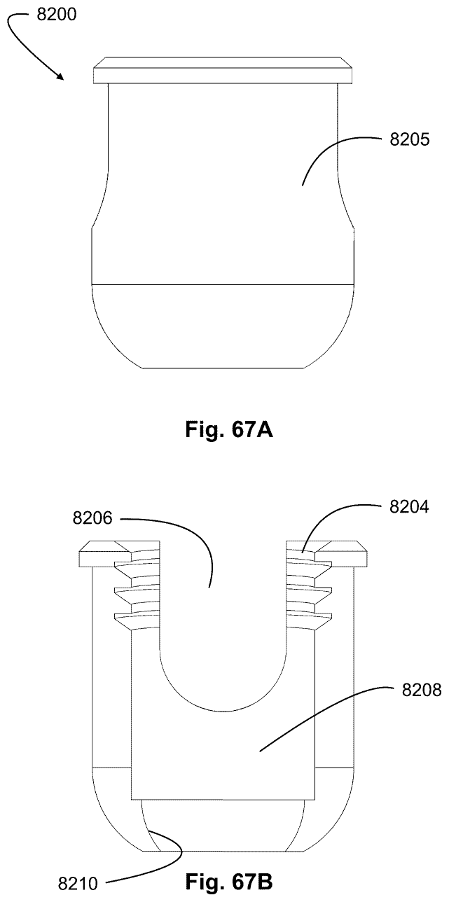

FIG. 67A is a side view of the tulip of FIG. 64A.

FIG. 67B is a side section view of the tulip of FIG. 64A.

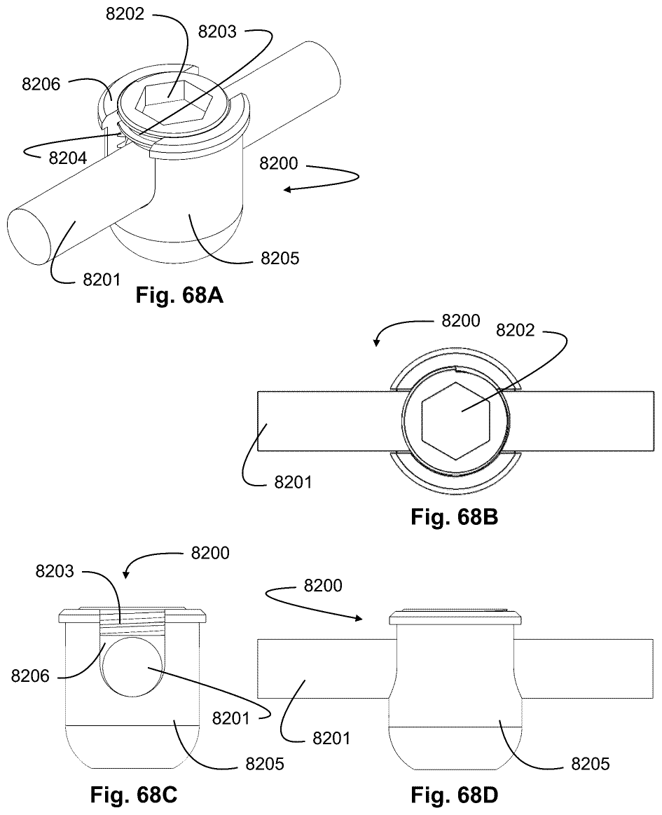

FIG. 68A is a perspective view of a tulip with a rod and a set screw according to an embodiment of the present disclosure.

FIG. 68B is a top view of the tulip with the rod and the set screw of FIG. 68A.

FIG. 68C is a front view of the tulip with the rod and the set screw of FIG. 68A.

FIG. 68D is a side view of the tulip with the rod and the set screw of FIG. 68A.

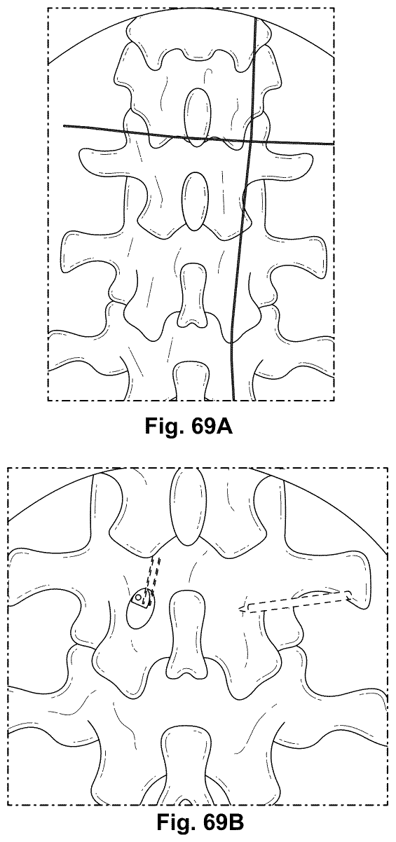

FIGS. 69A-77 are perspective views of steps in a method for facet fixation according to an embodiment of the present disclosure.

### DETAILED DESCRIPTION

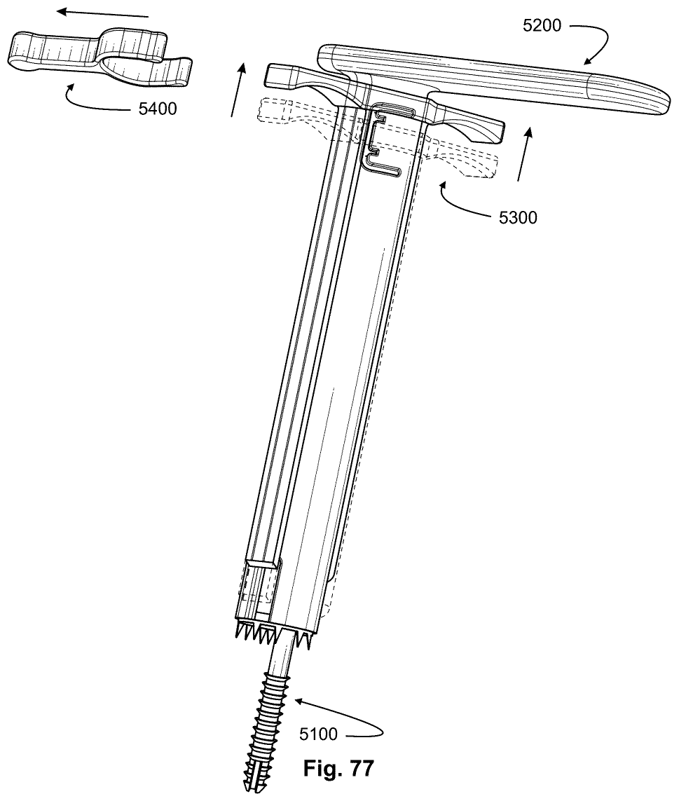

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIGS. 1A through 77, is not intended to limit the scope of the claims, but is merely representative exemplary of exemplary embodiments of the disclosure.

Those of skill in the art will recognize that the following description is merely illustrative of the principles of the disclosure, which may be applied in various ways to provide many different alternative embodiments and may be applicable outside the fields of surgery or medical devices. While the present disclosure is made in the context of facet joints in the lumbar spinal region for the purposes of illustrating the concepts of the design, it is contemplated that the present design and/or variations thereof may be suited to other uses, such as cervical facet joints, thoracic facet joints, other joints in the human body, or to stabilize bone fractures, etc. Moreover, the implants, instrumentation and methods set forth herein may be used in open, percutaneous, and/or minimally invasive procedures and may be placed via intra-facet, trans-facet, trans-laminar, or trans-pedicle means.

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. Ipsilateral means on the same side of the body. Contralateral means on the opposite side of the body. These descriptive terms may be applied to an animate or inanimate body.

The system and method of use in accordance with the present application may overcome one or more of the above-discussed problems commonly associated with conventional bone and joint fixation and instrumentation and methods for preparation and implantation of these devices. Specifically, facet fixation devices and ancillary instrumentation presented herein may enable facet fixation implants to be more efficiently and accurately placed. These and other unique features of the system and method of use are discussed below and illustrated in the accompanying drawings.

Not every feature of each embodiment may be labeled in every figure in which that embodiment appears, in order to keep the figures clear. Similar reference numbers (for example, those that are identical except for the first numeral) may be used to indicate similar features in different embodiments.

Any of the devices described herein may be fabricated from metals, alloys, polymers, plastics, ceramics, glasses, composite materials, or combinations thereof, including but not limited to: ABS, polycarbonate, glass filled polycarbonate, PEEK (polyether ether ketone), titanium, titanium alloys, commercially pure titanium grade 2, ASTM F67, Nitinol, cobalt chrome, stainless steel, UHMWPE (ultra-high molecular-weight polyethylene) and biodegradable materials, among others. Barium sulfate may be added to polymers, plastics, ceramics and/or composite materials to increase radio-opacity. Different materials may be used within a single part. The implants disclosed herein may also encompass a variety of surface treatments or additives to encourage bony attachment, including but not limited to: porous coatings, hydroxyapatite, TCP (tricalcium phosphate), anti-microbial additives, analgesics, anti-inflammatories, PRP (platelet-rich plasma), BMPs (bone morphogenic proteins), PMA (phorbol myristate acetate) material, bone growth promoting material, PLLA (poly-L-lactide), PGA (polyglycolide), TCP (tricalcium phosphate), demineralized bone, cancellous bone chips, etc. Any implant disclosed herein may include a subtractive etching process that may result in microscopic cuts in a surface of an implant. A subtractive etching process may promote in growth of bony material into an implant. Any implant disclosed herein may include a radiographic marker for imaging purposes. Any implant disclosed herein may be colored, coded or otherwise marked to make it easier for the surgeon to identify the type and size of the implant.

Any implant or instrument disclosed herein may be manufactured through additive manufacturing techniques, such as 3D printing, subtractive manufacturing techniques, such as machining, or other manufacturing techniques known in the art, such as injection molding and/or forging. Any implant disclosed herein may be manufactured with a lattice structure or a combination of generally solid portions and portions with a lattice structure. A lattice structure may be included on a bottom surface, a top surface and/or through an implant. A lattice structure may be configured to promote bone in growth and may comprise: a cell type stochastic lattice; a node type spherical lattice; a connector type cylinder lattice; a cell size 0.4 mm to 1.2 mm, preferably 0.8 mm to a side lattice; a connector diameter 0.1 mm to 0.2 mm, preferably 0.15 mm lattice; or any combination of lattices.

The present disclosure relates to bone and joint fixation and instrumentation and methods for preparation and implantation of these devices. Joint fixation may be necessary in cases of pain and inflammation due to cartilage degeneration, nerve impingement, spinal misalignment, and motion instability. The primary examples described herein illustrate how this concept may be applied to a vertebral facet joint, but this concept may apply equally to other joints where similar causes of pain and inflammation are indicated.

The facet fixation system disclosed herein may employ a simple pedicle targeting trajectory. The cap geometry may reduce placement sensitivity and may allow a surgeon to capture and compress an increased bone surface, while optionally lateralizing the implant to better accommodate interspinous process access.

FIG. 1A is a top perspective view of a cap 10 in accordance with an embodiment of the present disclosure. FIG. 1B is a bottom perspective view of the cap 10, FIG. 1C is a bottom view of the cap 10, and FIG. 1D is a top view of the cap 10. The cap disclosed herein may be referred to as a cap or a washer.

The cap 10 may have a first portion 16 and a second portion 17. Referring to FIG. 1D, the first portion 16 may be longer than the second portion 17 along a first axis 1 which intersects the first portion, the second portion, and an aperture 19 formed in the cap 10. In some embodiments, the first portion 16 may also be longer then the second portion 17 along a second axis 2. This may create an eccentrically shaped cap 10 with the first portion 16 being asymmetrically shaped in comparison to the second portion 17.

The shape of the cap 10 may also be referred to as “oblong”, with the first portion 16 forming a lobe that is larger than the second portion 17. The eccentric shape of the cap 10 may allow a surgeon more freedom to orient the larger lobe portion across the joint to facilitate joint fixation and may increase the load bearing area of the cap 10.

FIG. 1E is a back view of the cap 10, FIG. 1F is a front view of the cap 10, FIG. 1G is a left-side view of the cap 10, and FIG. 1H is a right-side view of the cap 10. In some embodiments, the cap 10 may also curve downward to create a lower profile implant. For example, FIG. 1G and FIG. 1H show left and right-side views of the cap 10 with the first portion 16 of the cap curving downward.

The cap 10 may include one or more teeth 14 on a bone engaging side 3 of the cap 10. The one or more of teeth 14 may have beveled surfaces 15 that may be arranged to, at least partially, oppose each other between the first portion 16 of the cap 10 and the second portion 17 of the cap 10. The beveled surfaces 15 may be made to diverge away from each other in the superior to inferior direction and converge toward each other in the inferior to superior direction. In this manner, the beveled surfaces 15 may act to compress the joint bones together as the one or more teeth 14 are driven into the joint bones.

The angle of the beveled surfaces 15 may be adjusted to increase or decrease the compressive forces created by the cap 10. For example, if the angle of the beveled surfaces 15 is increased, the teeth may impart a greater compressive force for a given distance the one or more teeth 14 are driven into the joint bones. Thus, the size, length, bevel shape, bevel angle, and distribution of the teeth may vary in any of the examples disclosed herein. For example, the number and spacing of the one or more teeth 14 may be chosen to maximize the fixation properties of the cap 10 in view of the size and condition of the joint bones of the patient.

The one or more teeth 14 may be distributed on the bone engaging side 3 of the cap 10 along the outer perimeter of the bone engaging side 3 of the cap 10. In other embodiments, the one or more teeth 14 may be distributed away from the outer perimeter of the bone engaging side 3 of the cap 10. For example, FIG. 1C has a tooth 4 which may not lie along the outer perimeter of the bone engaging side 3 of the cap 10, rather tooth 4 may be located deeper within the interior of the first portion 16. Having teeth distributed in this manner may increase the bone grabbing performance of the implant by increasing the number of teeth within the interior of the first portion 16.

Continuing with FIGS. 1A and 1D, the cap 10 may have an aperture 19 formed through the cap 10 and configured to receive a fastener 50, as can be seen in FIG. 5A and FIG. 5B. The shaft of the fastener 50 may be at least partially threaded to promote compression. The fastener 50 may also be self-tapping and may be cannulated down its center so that it may be placed into the vertebral facet joint along a K-wire.

The cap 10 may have a chamfered spherical capsule 12 shaped to receive a complimentarily shaped partially spherical fastener head 51, as shown in FIG. 5A. This may allow the fastener 50 to rotate within the aperture 19 and concentrically pivot along its longitudinal axis to ensure that the cap 10 can align itself with the joint bones as the cap 10 is forced into the joint bones. The aperture 19 may also be deep enough to allow the fastener head 51 to be recessed within the aperture 19 to provide a smooth, low-profile implant. A smooth, low-profile implant may help reduce irritation to surrounding soft tissue. The aperture 19 may also be encircled by a lip 13 which may project inward and may have a diameter slightly smaller in size than the diameter of the head 51 of the fastener 50. This may allow the fastener 50 to be “press fit” into the aperture such that the lip 13 provides an interference that may capture the fastener 50 within the aperture 19.

The lip 13 may be flush with the surrounding surface of the implant to avoid any abrupt changes in the shape of the implant resulting in smooth surfaces. Thus, the lip 13, that sits flush with the surrounding surface, may help reduce irritation to surrounding soft tissues, as compared to other interference fit configurations such as collet style interference structures which may have multiple slits and/or protruding structures that may cause interference and irritation to surrounding soft tissues and bones.

In another embodiment, a cap 10 may include a chamfered cylindrical capsule (not shown) shaped to receive a complimentarily shaped cylindrical fastener head. The axis of the chamfered cylindrical capsule may be aligned with an axis of the fastener.

Continuing with FIGS. 1A-1H, the cap 10 may have one or more recessed slots 18 formed in a surface of the cap 10. The one or more recessed slots 18 may interact with a guide tool to hold the cap 10 in a specific orientation during insertion. In other examples, the cap 10 may not include one or more one or more recessed slots 18 formed in a surface of the cap 10. One such example may be seen in FIG. 3.

FIG. 2A is a bottom perspective view of a cap 20 in accordance with an embodiment of the present disclosure. FIG. 2B is a bottom perspective view of a cap 21 according to an embodiment of the present disclosure. FIG. 2C is a bottom perspective view of a cap 22 in accordance with an embodiment of the present disclosure. FIG. 2D is a bottom perspective view of a cap 23 according to an embodiment of the present disclosure.

Each of the cap 20, cap 21, cap 22, and/or cap 23 may include one or more slots 24 configured to interact with a guide tool which may be configured to hold the cap 20, cap 21, cap 22, and/or cap 23 at a specific orientation during insertion. However, in other examples, the cap 20, cap 21, cap 22, and/or cap 23 may not include one or more slots 24. It will be appreciated that the location of the one or more slots 24 around the perimeter of the caps may vary, as may the size, diameter and/or number of the one or more slots 24. The one or more slots 24 may cooperate with a suitable guide for proper aligned placement of the cap 20, cap 21, cap 22, and/or cap 23 into the joint, as will be discussed in greater detail below. The cap 20, cap 21, cap 22, and/or cap 23 may also include a plurality of teeth 25 that may be cylindrical in shape and may have varying lengths. The plurality of teeth 25 may also include opposing beveled surfaces 26 similar to other embodiments disclosed herein.

FIG. 3 is side perspective view of a cap 30 in accordance with an embodiment of the present disclosure. FIG. 4 is top perspective view of a cap 40 in accordance with an embodiment of the present disclosure. The cap 40 is shown with an aperture or fenestration 41 formed through the cap 40 and configured to promote bone growth, or bone fusion, by providing a graft pocket for material such as bone chips or bone growth promoters.

FIG. 5A is a pre-assembled perspective view of an implant including the cap 10 and the fastener 50 according to an embodiment of the present disclosure. FIG. 5B is an assembled perspective view of the implant including the cap 10 and the fastener 50. FIG. 6 is a perspective view of an implant assembly including the cap 40 and a fastener 60 according to an embodiment of the present disclosure. The faster 60 may be fenestrated with apertures 61 throughout the fastener 60 which may also be packed with bone chips or bone growth promoters. This combination may further promote bony growth and bone fusion between the faster 60, the cap 40, and the joint bones.

FIG. 7A is a perspective view of a lumbar portion of a spine 70 with a facet fixation device 71 fastened to a vertebral facet joint according to an embodiment of the present disclosure. FIG. 7B is a posterior perspective view of the facet fixation device 71.

FIG. 8A is a perspective view of a lumbar portion of a spine with a facet fixation device 81 fastened to a vertebral facet joint in a first orientation according to an embodiment of the present disclosure. FIG. 8B is a perspective view of a lumbar portion of a spine with the facet fixation device 81 fastened to a facet joint in a second orientation. FIG. 8C is a perspective view of a lumbar portion of a spine with the facet fixation device 81 fastened to a facet joint in a third orientation. FIG. 8D is a perspective view of a lumbar portion of a spine with the facet fixation device 81 fastened to a facet joint in a fourth orientation. FIG. 8E is a perspective view of a lumbar portion of a spine with the facet fixation device 81 fastened to a facet joint in a fifth orientation.

FIGS. 8A-8E show examples of various placement options for the facet fixation device 81 in a lumbar portion of a spine 80, all of which may be easily achievable with the guides, instrumentation, and methods disclosed herein. FIG. 8A shows the facet fixation device 81 with the fastener piercing the lower or inferior part of the superior articular process and the lobe of the cap oriented superiorly to capture the inferior articular process with the lobe of the cap.

FIG. 8B shows the facet fixation device 81 with the fastener piercing the lateral or middle part of the superior articular process and the lobe of the cap oriented medially to capture the inferior articular process with the lobe of the cap. FIG. 8C shows the facet fixation device 81 with the fastener piercing the inferior articular process (transfacet) and the lobe of the cap inverted or oriented laterally to capture the superior articular process with the lobe of the cap.

FIG. 8D shows the facet fixation device 81 with the fastener piercing the upper or superior part of the superior articular process and the lobe of the cap oriented inferiorly to capture the inferior articular process with the lobe of the cap. FIG. 8E shows the facet fixation device 81 with the fastener piercing the inferior articular process (transfacet) and the lobe of the cap oriented medially to capture the inferior articular process with the lobe of the cap. It may be appreciated that any of the implants disclosed herein may be implanted in any of the orientations disclosed in FIGS. 8A-8E, including, but not limited to, facet fixation devices with circular footprints or perimeters.

FIG. 9 is a posterior perspective view of a cervical portion of a spine 90 with a facet fixation device 91 fastened to a facet joint in a cervical portion of a spine according to an embodiment of the present disclosure. FIG. 9 may demonstrate that the facet fixation devices disclosed herein may be used in all portions of the spine as well as in other parts of the body.

Methods of inserting the facet fixation devices disclosed herein will now be given. The disclosed methods may further include navigation and/or endoscopic methods known in the art. These methods may be used with any of the facet fixation devices including, but not limited to, cap 10, cap 20, cap 21, cap 22, cap 23, cap 30, cap 40, and facet fixation device 71, facet fixation device 81, facet fixation device 91, facet fixation device 100, facet fixation device 200, facet fixation device 300, and/or facet fixation device 400.

FIG. 10 is a perspective view of a dilator 500 in accordance with an embodiment of the present disclosure. FIG. 11 is a perspective view of a cannula 510 in accordance with an embodiment of the present disclosure. FIG. 12 is a perspective view of the dilator 500 inserted into the cannula 510.

A bone access needle may be used, along with fluoroscopy, to determine the desired location and trajectory of a facet fixation device. A K-wire may then be inserted through the bone access needle tube into a portion of the facet joint where the surgeon desires to affix a fastener to the facet joint. In one example, the K-wire may be inserted into an inferior facet joint and oriented such that the fastener may enter into the pedicle of the inferior facet joint.

Once the K-wire is in the desired location, the dilator 500, or series of sequential dilators (for example as shown in FIG. 69A), and the dilator 500 may be guided over the K-wire and inserted into the soft tissue of the patient to provide sufficient access to the facet joint. The dilator 500 may have a pointed tip 501 at its distal end 502 and a handle portion 503 at its proximal end 504. The dilator 500 may also have a shaft 505 having a diameter slightly less than the inside diameter of the hollow shaft 511 of a cannula 510 as seen in FIG. 11.

The dilator 500 may be inserted into the cannula 510 as shown in FIG. 12, and the handle portion 503 of the dilator 500 and the handle 512 of the cannula 510 may also align with and engage each other via a boss 506 attached to the handle portion 503 of the dilator 500 and an aperture 513 formed in the handle 512 of the cannula 510. Additionally, or alternatively, the handle portion 503 of the dilator 500 and/or the handle 512 of the cannula 510 may be configured as a T-handle.

The dilator 500 may be configured to be manufactured in two portions, generally split down a long axis of the dilator, and assembled into a single instrument prior to packaging and use.

In some embodiments, a cap may be eccentrically shaped with respect to a fastener, as a result, it may be beneficial to dilate soft tissue in a corresponding eccentric profile. A dilator 500 may include a through hole configured so that the dilator may be advanced along a k-wire into the soft tissue of a patient. Additionally, a longitudinal axis of the through hole may be parallel to but not coincident with a longitudinal axis of the dilator 500 so that, as the dilator 500 is advanced, the soft tissue may not be dilated equally in all directions relative to the k-wire.

Once the soft tissue is dilated, the dilator 500 may be removed from the cannula 510 thus exposing the facet joint through the cannula 510. A cannulated drill bit may then be advanced over the K-wire and may create a pilot hole for a fastener. After drilling to a desired depth, the cannulated drill bit may be removed leaving the k-wire in place within the bone.

A reamer 530 and/or a reamer 540 may then be used to ream the bone surface of the facet joint to prepare the bone surface for receiving the facet fixation device. The reamer 530 shown in FIG. 13 may be a manual reamer with a handle 531 and a reamer head 532. The reamer 540 shown in FIG. 14 may be a powered reamer with a connection 541 configured to receive a suitable power tool and a reamer head 542.

FIG. 15A is a perspective view of a guide 550 in accordance with an embodiment of the present disclosure. FIG. 15B is a front view of the guide 550. Once the implant site is sufficiently prepared to receive the facet fixation device, one or more guides may be used to orient and insert a facet fixation device, as can be seen in FIGS. 15A-17C.

FIGS. 15A-15B illustrate one example of a guide 550 that may be used with a cap 30 shown in FIG. 3. The cap 30 does may have any slots to engage a portion of the guide 550, as other embodiments disclosed herein. Rather, the guide 550 may shaped to receive the smaller second portion of the cap 30 in the smaller inner portion 551 of the guide 550 and the larger first portion of the cap 30 in the larger inner portion 552 of the guide 550, as is shown in the front view of the guide in FIG. 15B. The guide 550 may a semi-tubular or semi-cylindrical member. The outer diameter of the guide 550 may be round to complementarily fit within the cannula 510, but the inner diameter may have a unique cutout profile to accommodate a cap with a variable diameter, such as cap 10, cap 20, cap 30, and/or cap 40. In the example shown, the guide 550 may not be quite a half-pipe as it sweeps close to 245°. Other guide examples may vary in size and shape to accommodate the geometry of other cap embodiments, including cap 10, cap 20, cap 21, cap 22, cap 23, cap 40, cap 102, cap 202, cap 302, cap 402, and/or cap 402′.

The inner diameters of the cannula 510 and guide 550 may match the two different outer diameters of the cap 30; other guides may match the outer diameters of the lobes or perimeters of the other caps. This may provide control for proper placement of the cap 30. Thus, the guide 550 may be shaped to cooperate with the asymmetrical or eccentric geometry of the cap 30 to guide the cap 30 into place. The guide 550 may be inserted into the cannula 510 and a cap 30 with a fastener attached thereto may be affixed to a driver 580, such as that shown in FIG. 18. The cap 30 may then be inserted into the guide 550 and moved toward the implant site.

The shape of the guide 550 in combination with the shape of the cap 30 may retain the cap 30 in a proper orientation as the cap 30 slides toward the implant site, and as the fastener is driven into the bone. Caps having a circular base perimeter such as cap 102, cap 202, cap 302, cap 402, and/or cap 402′ may be implanted with a guide 550 having a circular inner diameter; alternately the guide 550 may not be used and a cap may be implanted directly through the cannula 510.

The driver 580 can have a hexagonal tip 581 configured to interact with a hexagonal aperture 52 as seen in FIG. 5A. The hexagonal aperture 52 can also be chamfered to help the driver 580 stay engaged with the fastener 50.

In one method of implantation, the cap 30 may be placed first, allowing the teeth to capture bone surfaces on both sides of the joint, followed by placement of the fastener 50 to provide compression and stability. As the fastener head 51 engages in spherical capsule 12, the teeth 14 are driven into the bone; the beveled surfaces 15 of the teeth promote compression across the facet joint.

FIG. 16A is a perspective view of a guide 560 in accordance with an embodiment of the present disclosure. FIG. 16B is a perspective view of the guide 560 with a cap 10 inserted into the guide according to an embodiment of the present disclosure. FIG. 16C is a perspective view of the guide 560 with a cap 10 and a fastener 50 inserted into the guide according to an embodiment of the present disclosure.

The guide 560 may have a hollow shaft 561 and a handle 562 with a boss 563 on the handle 562. The hollow shaft 561 may be hollow and may include one or more retaining members 564 engaged with the distal end of the hollow shaft 561. Moreover, the retaining members 564 may include boss members 565 sized and shaped to engage suitably shaped recessed slots 18 formed in the cap 10. In other examples, the cap 10 may include other features to cooperate with the one or more retaining members 564, such as recesses, dimples, or grooves. The boss members 565 may be oriented to be offset from each other at the distal end of guide 560 in order to match the offset or eccentric shape of the slots 18 in the cap 10.

Guide 560 may also be used to implant the facet fixation device 100, the facet fixation device 200, the facet fixation device 300, and/or the facet fixation device 400; the boss members 565 may be received in the connection features of the cap 102, the cap 202, the cap 302, and/or the cap 402 to guide the cap toward the implantation site and hold the facet fixation device while the fastener 104, the fastener 204, the fastener 304, and/or the fastener 404 is engaged in the bone.

FIG. 17A is a perspective view of a guide 570 in accordance with an embodiment of the present disclosure. FIG. 17B is a perspective view of the guide 570 with a cap 20 inserted into the guide according to an embodiment of the present disclosure. FIG. 17C is a perspective view of the guide 570 with a cap 20 and a fastener 50 inserted into the guide according to an embodiment of the present disclosure. The guide 570 may have a hollow shaft 571, a handle 574, and a guide pin 572.

The guide pin 572 may be offset from a central longitudinal axis of the guide 570. The guide pin 572 may cooperate with the slot 24 on the cap 20 to guide the cap 20 along a selected path into proper alignment with the joint. The guide pin 572 may also include a tip 573 which may act as a probe to aid in referencing a joint space. FIG. 17B shows the guide 570 with a cap 20 engaged with the guide 570 via a driver 580 and the guide pin 572. FIG. 17C shows the guide 570 engaged with the fastener 50 and the cap 20 with the driver 580 pushing the cap 20 and the fastener 50 in the distal direction along the guide pin 572.

FIG. 18 is a perspective view of a driver 580 in accordance with an embodiment of the present disclosure. The driver 580 may include a hexagonal tip 581 configured to engage a fastener. In other examples, the hexagonal tip 581 may be replaced with another shaped drive feature for connection with a fastener. Any of the cap embodiments disclosed herein may include slots 24 to allow use of guide 570 during an implantation procedure.

All of the above guides may be used to orient, steer, and insert a facet fixation device to the desired location at the implant site where the driver 580 may then be used to apply a torsional rotational force to the fastener 50 to fasten the facet fixation device to the joint to stabilize the joint. Once the facet fixation device is in the proper location, the guide, the driver 580, the cannula 510, and/or the K-wire may be removed and the incision site may be closed.

FIG. 19A is a perspective view of a facet fixation device 100 having a cap 102 with a circular footprint and a fastener 104 according to an embodiment of the present disclosure. The facet fixation device 100 and/or the other facet fixation devices herein may be referred to as an implant. When implanted, fastener 104 may extend through the cap 102 and through a facet joint, fastening the cap 102 to the facet joint and stabilizing the joint. The cap 102 may span the joint, with a portion of the cap 102 affixed to the inferior articular process of a facet joint, and another portion of the cap 102 affixed to the superior articular process of the facet joint. The width of the cap 102 may provide increased load bearing area for the fastener 104.

FIG. 20A is a perspective view of the cap 102 according to an embodiment of the present disclosure. FIG. 20B is a side view of the cap 102, FIG. 21A is a top view of the cap 102, and FIG. 21B is a bottom view of the cap 102.

The cap 102 may include a first side 110 and a second side 112, which may be superior and inferior sides, respectively. The first side 110 may be smoothly rounded to provide a low and minimally obtrusive device profile. The second side 112 may include an attachment surface 114. In the embodiment shown, the attachment surface 114 may be circular, and planar, although in other embodiments the attachment surface may be non-planar. An aperture 116 may extend through the cap 102 between the first side 110 and the second side 112. A cap axis 103 is centrally located relative to the aperture 116. The aperture 116 may also be circular, although in other embodiments the aperture 116 may take another shape.

The aperture 116 may be offset, or eccentrically positioned relative to the geometric center of the cap 102 and the outer circular footprint of the cap. Aperture 116 may be circumscribed by an aperture wall 117, which may be spherically concave or cup-shaped to receive the head of fastener 104. In other embodiments, the aperture wall may be flat, cylindrical, or conically concave. Toward the first side 110, the aperture wall 117 may widen at a first flared end 118 and toward the second side 112, the aperture wall 117 may widen at a second flared end 119, best seen in FIG. 19B. In another embodiment, cap 102 may include a second aperture 41 which may be a fenestration to provide a graft pocket for material such as demineralized bone material, graft material, bone chips and/or bone growth promoters.

The cap 102 may further include a circular base portion 120 and a raised portion 122. The circular base portion 120 may be shaped as a circular flange with a circular perimeter, and the raised portion 122 may project superiorly from the circular base portion 120. In the context of this disclosure, a perimeter is a line bounding a cap portion at its largest extent, the line bounding a plane perpendicular to the cap axis. While the circular base portion 120 may be circular with a circular perimeter, the raised portion 122 may not be radially symmetrical and may have a non-circular, eccentric perimeter, and in the embodiment shown is ovoid, having a wider first end 125 and a relatively narrower second end 127. The first end 125 and second end 127 may be lobes of the circular base portion 120; the widths of the first end 125 and the second end 127 may be measured in a plane perpendicular to the cap axis 103.

The second end 127 (or lobe) may encompass the aperture 116. Toward its juncture with the circular base portion 120, the raised portion 122 may include a flared section 124 which may extend around at least a portion of the raised portion 122. The flared section 124 may provide a concavely curved transition between the circular base portion 120 and the raised portion 122. As best seen in FIG. 20A and FIG. 21A, the circular base portion 120 may project radially outward beyond the raised portion 122 in every direction normal to cap axis 103. This shape may allow the base portion to provide a maximum footprint area for attachment to the facet joint and compression across the joint, while the raised portion may be reduced in size from the base portion, providing a minimally obtrusive device profile.

At least one connection feature which may be a recess may be present on the cap 102. The cap 102 may include two connection features 126, 128 which may be recessed into the raised portion 122, and may provide a location for an instrument to grasp the cap 102. Connection features 126, 128 may cooperate with retaining members 564 of guide 560 allowing guide 560 to guide placement of cap 102 in an implantation procedure. In alternate embodiments, the connection features 126, 128 may be formed as slots in communication with the aperture 116, or as protruding bosses or tabs. The flared section 124 may also provide a recessed surface for instrument connection. In other embodiments, the cap 102 may include one or more slots 24 for connection to a guiding or implantation instrument.

The attachment surface 114 may include a plurality of teeth 130 projecting inferiorly from the cap 102. The teeth 130 may vary in number and in distribution. The teeth 130 may each include at least one bevel 132. The at least one bevel 132 may be oriented, or face interiorly toward, the center of the attachment surface 114. The positioning of the teeth 130 may provide compression across the joint as the cap 102 is affixed to the joint, as the beveled tooth surfaces on teeth 130 on one side of the joint may oppose, or face, beveled tooth surfaces on teeth 130 on the opposite side of the joint. Methods of implantation described above for cap 102 and fastener 50 also apply to all facet fixation devices described herein, including, but not limited to: facet fixation device 100, facet fixation device 200, facet fixation device 300, and/or facet fixation device 400.

Referring to FIG. 19A and FIG. 19B, cap 102 is shown with a fastener 104. The fastener 104 may include a head portion 140, a shaft portion 142, and may be cannulated with a bore 144 extending the length of the fastener. The head portion 140 may include a connection feature 146 which may be a hex connection or other type of connection which permits a driver to turn the fastener 104. The fastener 104 may be poly-axially adjustable relative to the cap 102, with the head portion 140 rotatable poly-axially within the aperture wall 117. The second flared end 119 may allow space for the poly-axial tilting of the fastener 104 relative to the longitudinal axis of the aperture 116 and the cap axis 103. The shaft portion 142 may be at least partially threaded, with threads 148 occupying a portion of the shaft portion 142. It is appreciated that in other embodiments of facet fixation device 100 and the other facet fixation devices described herein, the fastener 104 may be replaced with another type of fastener. For example, a screw, a rivet, a brad, a nail, a bolt, a post, a peg, a staple, an anchor, a line, a flexible member, a cable, a wire or another type of fastener known in the art may be used to provide fixation of the cap across a facet joint. Fastener 104 and/or another fastener may lock to the cap 102 to prevent backout of the fastener 104 or backout of the cap 102.

FIG. 22A is a perspective view of cap 202 having a spiral footprint according to an embodiment of the present disclosure. FIG. 22B is a side perspective view of the cap 202, FIG. 22C is a side view of the cap 202, FIG. 23A is a top view of the cap 202, and FIG. 23B is a bottom view of the cap 202. The cap 202 may be formed as a continuous spiral, or snake-like shape, having a first end 225 (or lobe) which winds around and partially overlaps a second end 227 (or lobe).

The spiral may be asymmetrically shaped, with an overall length of the cap 202 greater than an overall width. The cap 202 may include a cap body 208 having a first side 210 opposite a second side 212; the cap may also have an interior wall 211 opposite an exterior wall 213. The interior wall 211 and exterior wall 213 may be generally perpendicular to the second side 212. As seen best in FIG. 23A and FIG. 23B, the thickness, or width w1, of the cap body 208 between the interior wall 211 and the exterior wall 213 may be constant throughout the cap body; in other embodiments the width of the cap body may vary at different locations. The exterior wall 213 may be smoothly rounded to provide a low unobtrusive profile when the cap 202 is implanted.

The cap 202 may further include an attachment surface 214 having a plurality of teeth 230 with bevels 232. As with cap 102, the positioning of the teeth 230 and bevels 232 may promote compression across the facet joint when the cap 202 is implanted with some teeth 230 on one side of the joint, and the remaining teeth 230 on the other side of the joint.

An aperture 216 having a surrounding aperture wall 217 may be formed in the second end 227. The aperture 216 may not be entirely closed off but a first gap 219 may be formed in the surrounding aperture wall 217. First and second connection features 226, 228 may be formed as recesses in the cap body 208, on the exterior wall 213. It may be appreciated that one or more connection features 226, 228 may be formed on any part of the cap body 208 in any location. In alternate embodiments, the connection features 226, 228 may be formed as slots in communication with the aperture 216, or as protruding bosses or tabs. In other embodiments, the cap 202 may include one or more slots 24 for connection to a guiding or implantation instrument. A second gap may be formed between the first end 225 and the second end 227. The first gap 219 and the second gap 221 may be referred to as fenestrations, and provide space for insertion of bone graft material.

FIG. 24A is a perspective view of a cap 302 having a circular footprint according to an embodiment of the present disclosure. FIG. 24B is top view of the cap 302 and FIG. 24C is a side view of the cap 302. Like facet fixation device 100, a cap 302 may have a circular footprint. The cap 302 may share many of the characteristics of the cap 102, which are numbered similarly, including a first side 310, second side 312, an aperture 316, an aperture wall 317, an attachment surface 314, a base portion 320, a raised portion 322, a flared section 324, beveled teeth 330, and connection features 326, 328, among others. Similar features shown and described for the cap 102 may be assumed to also apply to the cap 302.

While the base portion 320 may be circular, the raised portion 322 may not be radially symmetrical and may have a non-circular, eccentric perimeter. The shape of the raised portion 322 may differ from the shape of the raised portion 122 of cap 102 in that the relative sizes of the lobes of the raised portion may be reversed. A second end 327 (or lobe), which may encompass aperture 316, may be wider than a first end 325 (or lobe). The raised portion 322 may have a non-circular, eccentric perimeter, while the base portion 320 may have a circular perimeter. In alternate embodiments, the connection features 326, 328 may be formed as slots in communication with the aperture 316, or as protruding bosses or tabs.

In other embodiments, the cap 302 may include one or more slots 24 for connection to a guiding or implantation instrument. In alternate embodiments, the cap 302 may include a second aperture 41 which may be a fenestration to provide a graft pocket for material such as demineralized bone material, graft material, bone chips and/or bone growth promoters.

FIG. 25A is a perspective view of a cap 402 having a circular footprint according to an embodiment of the present disclosure. FIG. 25B is a top view of the cap 402, FIG. 25C is a side view of the cap, and FIG. 25D is a bottom view of the cap. Like the cap 102 and the cap 302, the cap 402 may have a circular footprint. The cap 402 may share many of the characteristics of the cap 102, which are numbered similarly, and may include a first side 410, a second side 412, an aperture 416, an aperture wall 417, an attachment surface 414, a base portion 420, a raised portion 422, a flared section 424, beveled teeth 430, a first end 425, a second end 427, and connection features 426, 428, among others. Similar features shown and described for cap 102 may be assumed to also apply to the cap 402.

The shape of the raised portion 422 may differ from the shape of the raised portion 122 of the cap 102 in that the raised portion 422 may be wider overall, for example between the connection features 426, 428, compared to raised portion 122 of the cap 102. The raised portion 422 of cap 402 may overlay more of the base portion 420 than does the raised portion 122 of the circular base portion 120 of the cap 102. The raised portion 422 may be described as wider or beefier than raised portion 122. The raised portion 422 may have a non-circular eccentric perimeter, while base portion 420 may have a circular perimeter. A flared section 424 of cap 402 may be less dramatically curved than the flared section 124 of cap 102. In alternate embodiments, the connection features 426, 428 may be formed as slots in communication with the aperture 416, or as protruding bosses or tabs. In other embodiments, the cap 402 may include one or more slots 24 for connection to a guiding or implantation instrument. In alternate embodiments, the cap 402 may include a second aperture 41 which may be a fenestration to provide a graft pocket for material such as demineralized bone material, graft material, bone chips and/or bone growth promoters.

Referring to FIG. 25D, cap 402′ may be an alternative embodiment of cap 402. The cap 402′ may share many of the same features as cap 402; however, the number and pattern of beveled teeth 430 projecting from attachment surface 414 may differ. Cap 402′ may have two beveled teeth 430 on the first end 425 between an outer row of beveled teeth 430 and the aperture 416, creating an outer perimeter of beveled teeth 430 and an inner row of beveled teeth 430. In comparison, cap 402 may have only one beveled tooth between an outer row and the aperture 416. It may be appreciated that in any of the embodiments disclosed herein, the number and pattern of beveled teeth 430 may vary; beveled teeth 430 may be distributed in rows, groups, or singly; randomly or regularly; symmetrically or asymmetrically.

FIG. 26 is a perspective view of a facet fixation device 200 including the cap 202 and a fastener 204 according to an embodiment of the present disclosure. The fastener 204 may be the same as fastener 104 and as such will not be further described. The fastener 204 may be poly-axially adjustable relative to cap 202. The discontinuous spiral shape of cap 202 may allow some conforming of cap 202 to the targeted joint members when cap 202 is implanted. For example, the attachment surface 214 on the first end 225 may not remain coplanar with attachment surface 214 on the second end 227 as the facet fixation device 200 is implanted; some relative movement may occur between the first end 225 and the second end 227.

FIG. 27 is a perspective view of a facet fixation device 300 including the cap 302 and a fastener 304 according to an embodiment of the present disclosure. The fastener 304 may be the same as fastener 104 and as such will not be further described. The fastener 304 may be poly-axially adjustable relative to the cap 302.

FIG. 28 is a perspective view of a facet fixation device 400 including the cap 402 and a fastener 404 according to an embodiment of the present disclosure. The fastener 404 may be the same as the fastener 104 and as such will not be further described. The fastener 404 may be poly-axially adjustable relative to the cap 402.

FIG. 29A is a top perspective view of a cap 3102 in accordance with an embodiment of the present disclosure. FIG. 29B is a bottom perspective view of the cap 3102, FIG. 29C is a side view of the cap 3102, and FIG. 29D is a front section view of the cap 3102. The cap 3102 may share many of the characteristics of the cap 102 and/or the cap 402, which may be numbered similarly, including, beveled teeth 3130, a first end 3125, a second end 3127, and connection features 3126, 3128, among others. Similar features shown and described for cap 102 and cap 402 may be assumed to also apply to cap 3102.

The cap 3102 may include an indicator button 3140 that may indicate when the cap 3102 is seated against the underlying bone surface. The indicator button 3140 may have a wide variety of configurations. In some embodiments, the indicator button 3140 may be a mechanical device that slides between a first position and a second position when the cap 3102 seats against the bone. The location of such a mechanical device may be visible during surgery, with and/or without the aid of other instruments or imaging technology. For example, the position of such an indicator button 3140 may be visible under fluoroscopy. Further, in some embodiments, the indicator button 3140 may be visible with the naked eye from a viewpoint proximal to the indicator base 3142 such that the implantation status of the cap 3102 may be readily confirmed during the implantation process.

As embodied in FIG. 29A, FIG. 29B, FIG. 29C, and FIG. 29D, the cap 3102 may include an indicator aperture 3141 through which the indicator button 3140 may be slidably captured. The first end 3125 may include a proximal surface 3146 and a distal surface 3148. The indicator aperture 3141 may pass through the proximal surface 3146 and the distal surface 3148. A second end 3127 may include an aperture 3116 configured to receive a fastener 3104. Additionally, or alternatively, the second end 3127 may include connection features 3126, 3128 that may facilitate connection to an inserter, as will be shown and described below.

The indicator button 3140 may include an indicator base 3142, an indicator ring 3143, and an indicator top 3144. Further, the cap 3102 may include an aperture ledge 3145 within the indicator aperture 3141. The aperture ledge 3145 may be configured so that the indicator button 3140 may be slidably captive between the first position and the second position. In the first position, the indicator ring 3143 may sit atop the aperture ledge 3145. The aperture ledge 3145 may be configured so that the indicator ring 3143 may not pass through. Moreover, in the first position, the indicator top 3144 may be flush with or below the proximal surface 3146. In the second position, the indicator base 3142 may contact the aperture ledge 3145. The aperture ledge 3145 may be configured so that an indicator base 3142 may not pass through. In the second position, the indicator top 3144 may be above the proximal surface 3146, providing clear visual evidence that the cap 3102 may be in close engagement with an underlying bone surface.

Further, in the second position, the indicator base 3142 may not protrude below a bevel teeth surface 3147. Additionally, or alternatively, the indicator button 3140 and the cap 3102 may be configured so that a distance between the first position and the second position is greater than the length of the beveled teeth 3130. Moreover, the indicator button 3140 and the cap 3102 may be configured so that when the indicator button 3140 is in the second position, the indicator base 3142 may not prevent the entire length of the beveled teeth 3130 from being inserted into a bony surface.

The indicator button 3140 and the cap 3102 may be configured so that the transition from the first position to the second position is clear to a user. The indicator button 3140 and the cap 3102 may be configured so that the indicator button 3140 travels from the first position to the second position as the facet fixation device 3100, and more specifically, the cap 3102, is advanced into engagement with a bony surface. More specifically, the indicator base 3142 may be incident on the underlying bony surface when the cap 3102 is partially inserted into the bony surface, and may transition from the first position to the second position as the beveled teeth 3130 are inserted into the bony surface.

The indicator button 3140 may be fixed at a first position with a frangible connection designed to break at a threshold pressure. For example, a weld joint between the indicator button 3140 and the main body of the cap 3102 may break free upon insertion of the cap 3102 into the bony surface. Additionally, or alternatively, the cap 3102 and the indicator button 3140 may be integrally manufactured using an additive manufacturing process. The frangible connection between the indicator button 3140 and the cap 3102 may be created as part of the additive manufacturing process. The indicator button 3140 and cap 3102 may be configured so that the indicator button 3140 is in a second position when the beveled teeth 3130 are seated within a bony surface.

In some embodiments, lattice structures, porous surfaces, surface roughening, and/or other features may be present on one or more surfaces of the cap 3102. Such features may help facilitate bony growth and/or on-growth on the cap 3102. Use of such features on the distal surface 3148 may enable the underlying bone to grow into the distal surface 3148. Additionally, or alternatively, use of such features on the proximal surface 3146 may facilitate bone growth over and/or into the proximal surface 3146 to further capture the cap 3102 against the bone, by forming a barrier to prevent loosening of the cap 3102 from the bone. Formation of such features on the proximal surface 3146 and the distal surface 3148 may further promote bone growth through the cap 3102, particularly if the cap 3102 is made with a hollow interior therein, in communication with the lattice structures of the proximal surface 3146 and the distal surface 3148.

FIG. 29E is a partial section view of the cap 3102 in accordance with an embodiment of the present disclosure. The distal surface 3148 and/or the proximal surface 3146 may have lattice structures formed thereon. The lattice structure may be hydrophilic. The lattice structures may be additive manufactured and deposited or otherwise added on existing surfaces of the cap 3102. In the alternative, the cap 3102, as a whole, may be additive manufactured, inclusive of the lattice structures on the distal surface 3148 and/or the proximal surface 3146. According to another embodiment, the cap 3102 may be manufactured conventionally (for example, via forging casting, milling, and/or other known methods), and then subtractive processes may be applied to form the lattice structures on the proximal surface 3146 and/or the distal surface 3148.

According to some embodiments, a first lattice structure 3180, may include a plurality of connectors 3170 that link a plurality of nodes 3174. A first lattice structure 3180 may further include cells 3171 within the first lattice structure 3180 created by empty space between the connectors 3170 and nodes 3174. The first lattice structure 3180 may be configured so that each end of the plurality of connectors 3170 terminates at one of the plurality of nodes 3174 or within a portion of solid material. An implant may include one or more non-contiguous regions of lattice structures.

The cap 3102 may include a first lattice structure 3180 and a second lattice structure 3175. The second lattice structure 3175 structure may be spaced apart from the first lattice structure 3180. The parameters, including but not limited to: cell type, node type, connector type cell size, and connector diameter, may be the same between the first lattice structure 3180 and the second lattice structure 3175. Alternately, the parameters, including but not limited to: cell type, node type, connector type cell size, and connector diameter, may be different between the first lattice structure 3180 and the second lattice structure 3175. The first lattice structure 3180 and/or the second lattice structure 3175 may be configured to promote bone growth. Additionally, or alternatively, the first lattice structure 3180 and/or the second lattice structure 3175 may be configured to be hydrophilic. Additionally, or alternatively, the first lattice structure 3180 and/or the second lattice structure 3175 may be configured to include cells 3171 that are configured to receive biologic material prior to implantation to promote bone growth after implantation. Additionally, or alternatively, the first lattice structure 3180 and/or the second lattice structure 3175 may be configured to include less metal mass implanted and more open areas for the bone to grow through the implant and create a more rigid fusion.

A facet fixation device may include a first lattice structure 3180 and a second lattice structure 3175. A generally solid portion 3173 may be present within the first lattice structure 3180 to increase mechanical strength of the implant. Further, load-carrying portion, e.g., a flared section 3150, may separate the first lattice structure 3180 and the second lattice structure 3175.

More precisely, parameters such as the following may be applied for generation of the lattice:
Cell type: stochastic;
Node type: spherical;
Connector type: cylinder;
Cell size: 0.8 mm to a side; and
Connector dia.: 0.15 mm.

These are merely exemplary settings. Any lattice generation, surface roughening, or pore generation techniques known in the art may be used to provide the desired surface characteristics for the proximal surface 3146 and/or the distal surface 3148.

FIG. 30A is a perspective view of a fastener 3104 according to an embodiment of the present disclosure. FIG. 30B is a perspective view of a facet fixation device 3100 in accordance with an embodiment of the present disclosure, including the cap 3102 and the fastener 3104. The facet fixation device 3100 may include a fastener 3104 and the cap 3102 described previously. Connection features 3126 and 3128 may be configured to facilitate connection of the facet fixation device 3100 to an inserter assembly 3500, which will be shown and described subsequently.

FIG. 30C and FIG. 30D show the indicator button 3140 in a first position; i.e., the indicator button 3140 has not been moved to the second position by engagement of the cap 3102 with the underlying bone. The fastener 3104 may include a cylindrical screw head 3163 including a first portion 3162 and a second portion 3160. The cylindrical screw head 3163 may be configured so that the second portion 3160 has a larger cross-sectional area than the first portion 3162. Additionally, the cap 3102 may include an aperture 3161 that may be configured so that the cross-sectional area of the aperture 3161 is larger than the cross-sectional area of the first portion 3162 and smaller than the cross-sectional area of the second portion 3160. The facet fixation device 3100 may be configured so that the fastener 3104 may press fit into the cap 3102 and, therefore, may be captive relative to the cap 3102 after assembly.

The fastener 3104 may be the same as fastener 104 and may include all the described features of a fastener 104. In the alternative, the fastener 3104 may be designed to facilitate bone growth. For example, the shank, head, and/or threading of the fastener 3104 may have a roughened surface, a porous surface, and/or an otherwise modified surface designed to facilitate bone growth into the fastener 3104.

In some embodiments, the fastener 3104 may be cast, machined, additive manufactured, and/or made through other conventional methods known in the art, and then during, and/or subsequently, processed to provide the desired surface roughness and/or porosity. For example, the fastener 3104 may subjected to etching and/or other subtractive processes that create pores, pits, troughs, knurling, and/or other surface features that encourage bone growth.

FIG. 31A is a side view of an inserter tube 3200 according to an embodiment of the present disclosure. FIG. 31B is a partial detail view of the inserter tube 3200 and FIG. 31C is an alternate side view of the inserter tube 3200. An inserter assembly 3500 may be used as a guiding instrument to implant any facet fixation device shown and described. The inserter assembly 3500 may include an inserter tube 3200, an inserter lockout 3300, and a removeable lock 3400. The inserter tube 3200 may include an inserter tube handle 3206 and a tube 3207. The tube 3207 may further include a slot 3201 and channels 3205, each through a wall of the tube 3207.

The slot 3201 may be configured to define a tab 3202. The tab 3202 may include a first locking prong 3203 and a second locking prong 3204. Further, the slot 3201 and the tab 3202 may be configured so that the tab 3202 may be deflected towards the inside of a tube 3207 when a force is applied to the first locking prong 3203 and/or the second locking prong 3204. Additionally, the slot 3201 and/or the tab 3202 may be configured so that the tab 3202 returns to its original position when the force is removed.

FIG. 32A is a side view of an inserter assembly 3500 according to an embodiment of the present disclosure. FIG. 32B is a partial detail view of the inserter assembly 3500 and FIG. 32C is an alternate side view of the inserter assembly 3500. The inserter assembly 3500 may include the inserter tube 3200 and an inserter lockout 3300. FIG. 33A is a front view of an inserter lockout 3300 according to an embodiment of the present disclosure. FIG. 33B is a top view of the inserter lockout 3300.

The inserter lockout 3300 may include an inserter lockout handle 3301 and arms 3302. Further, the inserter lockout handle 3301 may include a first bevel 3304 and a second bevel 3305. Additionally, arms 3302 may include guide tabs 3303. The inserter lockout handle 3301 may be configured to interact with the first locking prong 3203 and the second locking prong 3204 such that, as the inserter lockout handle 3301 travels along a tube 3207, there may be two captive positions that may limit the movement of the inserter lockout 3300. The first bevel 3304 and the second bevel 3305 may be configured to actuate the first locking prong 3203 and the second locking prong 3204 from a first captive position or locked position to a second captive position and from a second captive position to a first captive position. Each captive position may be overcome by applying a sufficient force on the inserter lockout handle 3301 along an axis of the tube 3207 in a desired direction. The first captive position may be a locked position in which the cap 3102 is not disengageable from the inserter assembly 3500. The second captive position may be an unlocked position in which the cap 3102 is disengageable from the inserter assembly 3500.

FIG. 34A is a perspective view of the inserter assembly 3500 and FIG. 34B is a partial bottom detail perspective view of the inserter assembly 3500. The guide tabs 3303 may be configured to slidably connect with channels 3205. The channels 3205 and guide tabs 3303 may be configured so that the distance the guide tabs 3303 may travel within channels 3205 may be the same or greater than the distance between the first captive position and the second captive position of the inserter lockout 3300 relative to the inserter tube 3200. The inserter tube 3200 may further include cap locking tabs 3208. The cap locking tabs 3208 may have a generally convex and/or circular profile and may include a lead-in angle. The cap locking tabs 3208 may be configured so that, when the inserter lockout 3300 is in the second captive position, the cap locking tabs 3208 may deflect outwards when the facet fixation device 3100 is inserted into the inserter tube 3200. The cap locking tabs 3208 may be further configured to engage the connection features 3126, 3128 in the cap 3102.

The inserter assembly 3500 may be further configured so that, when the inserter lockout 3300 is advanced to the first captive position, the arms 3302 may prevent the cap locking tabs 3208 from deflecting outwards. Additionally, the guide tabs 3303 may be configured to slidably engage an inner surface 3210 to prevent the arms 3302 from deflecting outwards. The inserter assembly 3500 and the facet fixation device 3100 may be configured so that when the cap locking tabs 3208 are engaged with the connection features 3126, 3128 and the inserter lockout 3300 is in the first captive position, the facet fixation device 3100 may be captive relative to the inserter assembly 3500.

Additionally, the inserter tube 3200 may include a ledge 3209. The ledge 3209 may be configured as a depth stop for the facet fixation device 3100, or more specifically the cap 3102, within the inserter tube 3200. Moreover, the ledge 3209 and the cap 3102 may be configured so that when the cap 3102 is captive relative to the inserter assembly 3500, the ledge 3209 may contact a portion of the cap 3102 that may be solid material, e.g., a flared section 3150. This may enable insertion of the facet fixation device 3100 into a bony surface without damage to lattice structures that may be present on the proximal surface 3146, as described above. The flared section 3150 may be configured as a load-carrying portion of material, within which the proximal lattice structure is absent, to permit insertion of the cap 3102 without transmitting impact force through the proximal lattice structure.

In another embodiment, an inserter assembly 3600 shown in FIG. 37A and FIG. 37B may include a ridge 3601. The ridge 3601 may include an aperture 3602 configured to allow access to a drive feature 3165 in the fastener 3104 when the facet fixation device 3100 is engaged with the inserter assembly 3600. Additionally, the inserter assembly 3600 may include a recessed portion 3603 that may be configured to receive the cap 3102. The inserter assembly 3600 may further include an edge 3604 configured to engage a flared section 3150 of the cap 3102. The edge 3604 may limit the distance the cap 3102 may be seated within the recessed portion 3603. Moreover, the edge 3604 may enable insertion of the facet fixation device 3100 into a bony surface without damage to lattice structures that may be present on the proximal surface 3146, as described above.

The recessed portion 3603 may be configured so that when the cap 3102 is fully seated within the recessed portion 3603 there is a first distance 3606 between the surface of the cap and the ridge 3601. Additionally, the recessed portion 3603 may be configured so that when the cap 3102 is fully seated within the recessed portion 3603 there is a second distance 3605 between the second portion 3160 of the fastener 3104 and the aperture 3116 of the cap 3102. The recessed portion 3603 may be configured so that the first distance 3606 is less than the second distance 3605 to prevent the fastener 3104 from disengaging with the cap 3102 during insertion of the facet fixation device 3100 into a bony surface, enabling rotation and linear translation between the fastener 3104 and the cap 3102.

FIG. 35A is a perspective view of the inserter assembly 3500, FIG. 35B is a perspective view of the inserter assembly 3500 including a facet fixation device 3100 according to an embodiment of the present disclosure. FIG. 35C is a perspective view of the inserter assembly 3500 including a facet fixation device 3100 according to an embodiment of the present disclosure. FIG. 35D is a perspective view of the inserter assembly 3500 including a facet fixation device 3100 and the removeable lock 3400 according to an embodiment of the present disclosure. FIG. 36 is a top view of a removeable lock 3400 according to an embodiment of the present disclosure. FIG. 37A is a side view of an inserter assembly 3500 according to an embodiment of the present disclosure. FIG. 37B is a partial detail side view of the inserter assembly 3600.

FIG. 35A, FIG. 35B, FIG. 35C and FIG. 35D show steps for loading a facet fixation device 3100 onto an inserter assembly 3500. FIG. 36 shows a removeable lock 3400 that may be used in a loading process. The removeable lock 3400 may include an inner surface 3401 that is configured to attach to the inserter tube 3200.

FIG. 35A shows the inserter assembly 3500 in a second captive position. In the second captive position, the inserter lockout handle 3301 may be advanced towards the inserter tube handle 3206. In the second captive position, both the first bevel 3304 and the second bevel 3305 may be located between the first locking prong 3203 and the inserter tube handle 3206.

FIG. 35B shows the facet fixation device 3100 inserted into the inserter assembly 3500. The cap locking tabs 3208 may be engaged with the connection features 3126, 3128.

FIG. 35C shows the inserter assembly 3500 in a first captive position, with the facet fixation device 3100 secured to the inserter assembly 3500. In the first captive position, the inserter lockout 3300, or more specifically the arms 3302, may prevent the cap locking tabs 3208 from deflecting outwards, thus ensuring that the facet fixation device 3100 is fully captured by the inserter assembly 3500. In the first captive position, the inserter lockout handle 3301 may be advanced away from the inserter tube handle 3206. Further, in the first captive position, both the first bevel 3304 and the second bevel 3305 may be located between the first locking prong 3203 and the second locking prong 3204.

FIG. 35D shows the inserter assembly 3500, with the facet fixation device 3100 secured to the inserter assembly 3500 and a removeable lock 3400 applied to the inserter assembly 3500. The removeable lock 3400 may be configured so that the width of the removeable lock 3400 is generally the same as the linear distance between the inserter lockout handle 3301 and the inserter tube handle 3206 with the inserter assembly 3500 in the first captive position. The removeable lock 3400 may be configured to prevent the inserter assembly 3500 from shifting out of the first captive position when the inserter assembly 3500, or more specifically the inserter tube handle 3206, is impacted during insertion of the facet fixation device 3100 into engagement with the bony surface.

FIG. 38A is a perspective view of a reamer 3700 according to an embodiment of the present disclosure. FIG. 38B is a partial detail perspective view of the reamer 3700. FIG. 38C is a perspective view of a reamer 3700 and k-wire 3800 assembly according to an embodiment of the present disclosure. FIG. 38D is a partial detail perspective view of the reamer 3700 and k-wire 3800 assembly.

In some embodiments, the reamer 3700 shown in FIGS. 38A-38D may be used to prepare the bone surface prior to insertion of the facet fixation device 3100 into the bone. The reamer 3700 may include a guide channel 3701 that may be configured to receive a k-wire 3800. The guide channel 3701 may be configured to retain the k-wire 3800 in position after the k-wire 3800 is seated within the guide channel 3701. Further, the guide channel 3701 may be offset from a longitudinal axis of the reamer 3700. The reamer cutting head 3705 may include an aperture 3702 that is configured to allow the k-wire 3800 to pass through the reamer cutting head 3705 and into the bone surface. The reamer cutting head 3705 may include a plurality of cutting edges 3703 extending radially from the center axis. The reamer cutting head 3705 may further include a long guide point 3704 configured to keep the reamer cutting head 3705 centered during the reaming process.

FIG. 39A is a perspective view of a reamer 3700 according to an embodiment of the present disclosure. FIG. 39B is a top view of the reamer 3700. The reamer 3700 may include a handle 3711, a shaft 3712, and a head 3713. Additionally, the reamer 3700 may include cannulation 3714 that may extend through the handle 3711, the shaft 3712, and the head 3713 and may be configured to allow passage of a k-wire 3800 through the reamer 3700. The handle 3711 may further include a non-circular recess 3710.

FIG. 40A is a perspective view of a drill 4000 according to an embodiment of the present disclosure. FIG. 40B is a top view of the drill 4000. FIG. 41 is a perspective view of a power adapter 4100 according to an embodiment of the present disclosure.

The power adapter 4100 may include a first end 4102, a second end 4101, and a cannulation 4103 that may extend through the length of the power adapter 4100. The first end 4102 may be configured to facilitate connection of the power adapter 4100 to a powered drill chuck. The first end 4102 may be configured with a triangular cross section or other geometry that may operably engage with a three-jaw-chuck. The second end 4101 may be configured to engage a non-circular recess 3710 included in the reamer 3700. The second end 4101 may be configured with a non-circular cross section such as square, rectangular, hex, hexalobe or other non-circular geometry. The second end 4101 may further be configured with a taper on one or more of the faces, or other connection feature that results in the power adapter 4100 being releasably captive relative to the non-circular recess 3710 of the handle 3711.

The reamer 3700 and power adapter 4100 may be configured so that when the power adapter 4100 is engaged in the non-circular recess 3710 in the handle 3711, the cannulation 4103 of the power adapter 4100 and the cannulation 3714 of the reamer 3700 may be aligned and facilitate passage of a k-wire 3800 through both the power adapter 4100 and the reamer 3700.

The drill 4000 may include a handle 4011, a shaft 4012, and a fluted portion 4013. Additionally, the drill 4000 may include cannulation 4014 that may extend through the handle 4011, the shaft 4012, and the fluted portion 4013 and may be configured to allow passage of a k-wire 3800 through the drill 4000. The handle 4011 may further include a non-circular recess 4010. The second end 4101 of the power adapter 4100 may further be configured with a taper on one or more of the faces, or other connection feature that results in the power adapter 4100 being releasably captive relative to the non-circular recess 4010 of the handle 4011.

The drill 4000 and power adapter 4100 may be configured so that when the power adapter 4100 is engaged in the non-circular recess 4010 in the handle 4011, the cannulation 4103 of the power adapter 4100 and the cannulation 4014 of the drill 4000 may be aligned and facilitate passage of a k-wire 3800 through both the power adapter 4100 and the drill 4000.

FIG. 42A is a perspective view of a driver 4200 according to an embodiment of the present disclosure. FIG. 42B is a front view of the driver 4200 and FIG. 42C is a top view of the driver 4200. The driver 4200 may include a handle 4202, a connection portion 4201, a shaft 4203 and a tip 4204. Additionally, the driver 4200 may include cannulation 4214 that may extend through the handle 4202, the connection portion 4201, the shaft 4203, and the tip 4204 and may be configured to allow passage of a k-wire 3800 through the driver 4200. The tip 4204 may be configured to engage a drive feature 3165 of a fastener 3104. The connection portion 4201 may be configured to receive a universal tracker device (not shown) to facilitate the use of a surgical navigation system.

To prepare the bone surface using reamer 3700, the reamer 3700 may be advanced along a k-wire 3800 to the bone surface; the k-wire 3800 may then be retracted from the bone into the guide channel 3701; the reamer 3700 may be rotated until a desired amount of bone is removed from the surface and the implant site is sufficiently prepared; the reamer 3700 may be rotated to the original position; the k-wire 3800 may then be advanced along the guide channel back into the original hole in the bone created by the k-wire 3800; and the reamer 3700 may then be retracted along the k-wire 3800.

FIG. 43A is a perspective view of a facet fixation device 5100 according to an embodiment of the present disclosure. FIG. 43B is a top view of the facet fixation device 5100 and FIG. 43C is a side view of the facet fixation device 5100. The facet fixation device 5100 may include a fastener 5104 and a cap 5102. The facet fixation device 5100 may be configured so that the fastener 5104 may press fit into the cap 5102 and, therefore, may be captive relative to the cap 5102 after assembly. The facet fixation device 5100 may be utilized for bilateral transfacet fusion. Alternatively, the facet fixation device 5100 may utilized in unilateral pedicle screw fixation combined with contralateral percutaneous translaminar facet screw fixation

The fastener 5104 may be the same as fastener 3104 and may include all the described features of a fastener 3104. In the alternative, the fastener 5104 may be designed to facilitate bone growth. For example, the shank, head, and/or threading of the fastener 5104 may have a roughened surface, a porous surface, and/or an otherwise modified surface designed to facilitate bone growth into the fastener 5104. The fastener 5104 may include fenestrations to facilitate bone growth into the fastener 5104.

FIG. 44A is a perspective view of a cap 5102 according to an embodiment of the present disclosure. FIG. 44B is a top view of the cap 5102. FIG. 44C is a front view of the cap 5102, and FIG. 44D is a front view of the cap 5102. The cap 5102 may share many of the characteristics of the cap 3102 and/or the cap 102, which may be numbered similarly, including, beveled teeth 5130, a first end 5125, a second end 5127, and connection features 5126, 5128, among others. Similar features shown and described for cap 3102 and cap 102 may be assumed to also apply to cap 5102.

The cap 5102 may include an indicator button 5140 that may indicate when the cap 5102 is seated against the underlying bone surface. The indicator button 5140 may have a wide variety of configurations. In some embodiments, the indicator button 5140 may be a mechanical device that slides between a first position and a second position when the cap 5102 in response to engagement of the distal surface with the bone. The location of such a mechanical device may be visible during surgery, with and/or without the aid of other instruments or imaging technology. For example, the position of such an indicator button 5140 may be visible under fluoroscopy. Further, in some embodiments, the indicator button 5140 may be visible with the naked eye from a viewpoint proximal to an indicator base 5142 such that the implantation status of the cap 5102 may be readily confirmed during the implantation process.

The cap 5102 may include an indicator aperture 5141 through which the indicator button 5140 may be slidably captured. The first end 5125 may include a first lattice structure 5180 and a second lattice structure 5175. The second lattice structure 5175 may be spaced apart from the first lattice structure 5180. The indicator aperture 5141 may pass through the first lattice structure 5180 and the second lattice structure 5175. A second end 5127 may include an aperture 5116 configured to receive a fastener 5104. The aperture 5116 may be located offset from a geometric center of the cap 5102. Additionally, or alternatively, the second end 5127 may include connection feature 5126 and connection feature 5128 that may facilitate connection to an inserter, as will be shown and described below.

The indicator button 5140 may include an indicator base 5142, and an indicator top 5144. Further, the cap 5102 may include an aperture ledge 5145 within the indicator aperture 5141. The aperture ledge 5145 may be configured so that the indicator button 5140 may be slidably captive between the first position and the second position. The aperture ledge 5145 may be configured so that the indicator button 5140 may not pass through. Moreover, in the first position, the indicator top 5144 may be flush with or below the first lattice structure 5180. In the second position, the indicator base 5142 may contact the aperture ledge 5145. The aperture ledge 5145 may be configured so that the indicator button 5140 may not pass through. In the second position, the indicator top 5144 may be above the first lattice structure 5180, providing clear visual evidence that the cap 5102 may be in close engagement with an underlying bone surface.

Further, in the second position, the indicator base 5142 may not protrude below a bevel teeth surface 5147. Additionally, or alternatively, the indicator button 5140 and the cap 5102 may be configured so that a distance between the first position and the second position is greater than the length of the beveled teeth 5130. Moreover, the indicator button 5140 and the cap 5102 may be configured so that when the indicator button 5140 is in the second position, the indicator base 5142 may not prevent the entire length of the beveled teeth 5130 from being inserted into a bony surface.

The indicator button 5140 and the cap 5102 may be configured so that the transition from the first position to the second position is clear to a user. The indicator button 5140 and the cap 5102 may be configured so that the indicator button 5140 travels from the first position to the second position in response to engagement of the distal surface with the bone. More specifically, the indicator base 5142 may be incident on the underlying bony surface when the cap 5102 is partially inserted into the bony surface, and may transition from the first position to the second position as the beveled teeth 5130 are inserted into the bony surface.

The indicator button 5140 may be fixed at a first position with a frangible connection designed to break at a threshold pressure. For example, a weld joint between the indicator button 5140 and the main body of the cap 5102 may break free upon insertion of the cap 5102 into the bony surface. Additionally, or alternatively, the cap 5102 and the indicator button 5140 may be integrally manufactured using an additive manufacturing process. The frangible connection between the indicator button 5140 and the cap 5102 may be created as part of the additive manufacturing process. The indicator button 5140 and cap 5102 may be configured so that the indicator button 5140 is in a second position when the beveled teeth 5130 are seated within a bony surface.

In some embodiments, lattice structures, porous structures, and/or other features may be present on one or more surfaces of the cap 5102. Such features may help facilitate bony growth and/or on-growth on the cap 5102. Use of such features on the second lattice structure 5175 may enable the underlying bone to grow into the second lattice structure 5175. Additionally, or alternatively, use of such features on the first lattice structure 5180 may facilitate bone growth over and/or into the first lattice structure 5180 to further capture the cap 5102 against the bone, by forming a barrier to prevent loosening of the cap 5102 from the bone. Formation of such features on the first lattice structure 5180 and the second lattice structure 5175 may further promote bone growth through the cap 5102, particularly if the cap 5102 is made with a hollow space therein, in communication with the first lattice structure 5180 and the second lattice structure 5175.

The first lattice structure 5180 and the second lattice structure 5175 may be hydrophilic. The first lattice structure 5180 and the second lattice structure 5175 may be additive manufactured. The cap 5102, as a whole, may be additive manufactured, inclusive of the first lattice structure 5180 and the second lattice structure 5175. Alternatively, the cap 5102 may be manufactured conventionally (for example, via forging casting, milling, and/or other known methods), and then subtractive processes may be applied to form the first lattice structure 5180 and the second lattice structure 5175. The first lattice structure 5180 and the second lattice structure 5175 may be manufactured with parameters, and have features similar to, the first lattice structure 5180 and the second lattice structure 5175 previously described. Solid material, e.g., a flared section 5150, may separate the first lattice structure 5180 and a second lattice structure 5175.

FIG. 45A is a perspective view of a drill 6000 according to an embodiment of the present disclosure. FIG. 45B is a front view of the drill 6000. FIG. 46 is a side view of a k-wire 5800 according to an embodiment of the present disclosure. The drill 6000 may be configured to create a pilot hole in a bone to facilitate insertion of the fastener 5104 into the bone. The drill 6000 may include a handle 6011, a shaft 6012, and a fluted portion 6013. Additionally, the drill 6000 may include cannulation 6014 that may extend through the handle 6011, the shaft 6012, and the fluted portion 6013 and may be configured to allow passage of a k-wire 5800 through the drill 6000.

The handle 6011 may be sized and shaped to facilitate manual rotation of the fluted portion 6013 to drill into a portion of bone. The drill 6000 may be configured so that the fluted portion may be manufactured of metal, or another material with sufficient mechanical properties to cut bone. The handle 6011 and the shaft 6012 may be manufactured from injection moldable plastic. The drill 6000 may be configured so that the fluted portion 6013 may be insert molded into the handle 6011 and the shaft 6012.

The shaft 6012 may include a first depth indicator 6016 and a second depth indicator 6018. The first depth indicator 6016 may be configured so that, when the drill 6000 is advanced through a cannula 7200, the first depth indicator 6016 aligns with a handle 7202 of the cannula 7200, indicating a first depth the fluted portion 6013 has advanced into a bone. The first depth indicator 6016 may indicate a first drill depth of approximately 35 mm. The second depth indicator 6018 may be configured so that, when the drill 6000 is advanced through the cannula 7200, the second depth indicator 6018 aligns with the handle 7202 of the cannula 7200, indicating a second depth the fluted portion 6013 has advanced into the bone. The second depth indicator 6018 may indicate a second drill depth of approximately 50 mm.

FIG. 47A is a perspective view of an array 7000 according to an embodiment of the present disclosure. FIG. 47B is a top view of the array 7000, FIG. 47C is a front view of the array 7000, and FIG. 47D is a side view of the array 7000. The array 7000 may be configured to receive a driver 6200, specifically a connection portion 6201 of the driver 6200. Alternatively, the array 7000 may be configured to receive the drill 6000, specifically the shaft 6012 of the drill 6000. Alternatively, the array 7000 may be configured to receive any instrument of the facet fixation system. The array 7000 may be configured to facilitate real-time trajectory alignment and relative motion tracking of the drill 6000 by a surgical navigation system and/or a robotic assisted surgical system.

The array 7000 may include an inside diameter 7003, a plurality of locators 7001, a body 7002, and a plurality of arms 7004. The inside diameter 7003 may be configured to receive the connection portion 6201 of the driver 6200. Alternatively, the inside diameter 7003 may be configured to receive the shaft 6012 of the drill 6000. The inside diameter 7003 may be adjustable to accommodate instruments having varying diameters.

The plurality of locators 7001 may be positioned at the ends of the plurality of arms 7004. The plurality of locators 7001 may be configured as reference features to facilitate real-time trajectory alignment and relative motion tracking of the drill 6000 by a surgical navigation system and/or a robotic assisted surgical system. The body 7002 may include the inside diameter 7003.

FIG. 48A is a perspective view of a reamer 5700 according to an embodiment of the present disclosure. FIG. 48B is a front view of the reamer 5700, FIG. 48C is a bottom view of the reamer 5700, and FIG. 48D is a perspective view of the reamer 5700. The reamer 5700 may be used to prepare the bone surface prior to insertion of the facet fixation device 5100 into the bone. The reamer 5700 may include a handle 5711, a shaft 5712, and a reamer cutting head 5705. Additionally, the reamer 5700 may include cannulation 5701 that may extend through the handle 5711, the shaft 5712, and the reamer cutting head 5705 and may be configured to allow passage of a k-wire 3800 through the reamer 5700.

The shaft 5712 may include a first depth indicator 5716 and a second depth indicator 5718. The first depth indicator 5716 may have a height of approximately 2.5 mm. The second depth indicator may also have a height of approximately 2.5 mm. Additionally, a distance between the first depth indicator 5716 and the second depth indicator 5718 may be approximately 2.5 mm. As the reamer 5700 is advanced through the cannula 7200, the first depth indicator 5716 and the second depth indicator 5718 may be used to estimate the depth of bone removed by the reamer cutting head 5705.

The reamer cutting head 5705 may include an aperture 5702 aligned with the cannulation 5701. The aperture 5702 and the cannulation may be parallel to and offset from a longitudinal axis of the shaft 5712. Additionally, the aperture 5702 and the cannulation 5701 may not be concentric with the reamer cutting head 5705. The handle 5711 may include an indication arrow 5713. The indication arrow 5713 may indicate a direction from an aperture center point 5714 towards a head center point 5715. The indication arrow 5713 may be configured to align the reamer 5700 with a large dilator 7540 by aligning the indication arrow 5713 with a notch 7546 on the large dilator 7540.

The reamer cutting head 5705 may include a plurality of clockwise cutting edges 5703 extending radially from a longitudinal axis of the reamer 5700. The reamer cutting head 5705 may also include a plurality of counter-clockwise cutting edges 5704 extending radially from the longitudinal axis of the reamer 5700. The plurality of clockwise cutting edges 5703 may be configured so that at least one of the clockwise cutting edges 5703 cuts a portion of bone when the reamer 5700 is rotated in a clockwise direction. The plurality of counter-clockwise cutting edges 5704 may be configured so that at least one of the plurality of counter-clockwise cutting edges 5704 cuts a portion of bone when the reamer 5700 is rotated in a counter-clockwise direction. The reamer 5700 may optimally cut a portion of bone by oscillating the reamer 5700 ±45 degrees from a starting orientation and thereby cutting in both the clockwise and counter-clockwise directions.

Alternatively, the reamer cutting head 5705 may include a plurality of cutting edges extending radially from a longitudinal axis of the reamer 5700. Each one of the plurality of cutting edges may be configured to cut a portion of bone when rotated in clockwise direction and/or a counter-clockwise direction.

To prepare the bone surface using reamer 5700, the reamer 5700 may be advanced along a k-wire 5800 to the bone surface, the reamer 5700 may be oscillated ±45 degrees from a starting orientation until a desired amount of bone is removed from the surface and the implant site is sufficiently prepared, and the reamer 5700 may be retracted along the k-wire 5800.

Alternatively, to prepare the bone surface using reamer 5700, the reamer 5700 may be advanced along a k-wire 5800 to the bone surface; the k-wire 5800 may then be retracted from the bone into the cannulation 5701; the reamer 5700 may be oscillated ±45 degrees from a starting orientation until a desired amount of bone is removed from the surface and the implant site is sufficiently prepared; the reamer 5700 may be rotated to the original position; the k-wire 5800 may then be re-advanced through the cannulation 5701 back into the original hole created in the bone by the k-wire 5800; and the reamer 5700 may then be retracted along the k-wire 5800.

FIG. 49A is a perspective view of a driver 6200 according to an embodiment of the present disclosure. FIG. 49B is a front view of the driver 6200, FIG. 49C is a bottom view of the driver 6200, and FIG. 49D is a perspective view of the driver 6200. The driver 6200 may include a handle 6202, a connection portion 6201, a shaft 6203 and a tip 6204. Additionally, the driver 6200 may include cannulation 6214 that may extend through the handle 6202, the connection portion 6201, the shaft 6203, and the tip 6204 and may be configured to allow passage of a k-wire 5800 through the driver 6200. The tip 6204 may be configured to engage a drive feature 5165 of the fastener 5104. The connection portion 6201 may be configured to receive an array 7000 to facilitate the use of a surgical navigation system.

The tip 6204 may be configured with a non-circular cross section such as square, rectangular, hex, hexalobe or other non-circular geometry. The tip 6204 may further be configured with a taper on one or more of the faces, or other connection feature that results in the tip 6204 being releasably captive relative to the drive feature 5165 of the fastener 5104.

FIG. 50A is a perspective view of a first small dilator portion 7300 according to an embodiment of the present disclosure. FIG. 50B is a perspective view of the first small dilator portion 7300. FIG. 50C is a perspective view of a second small dilator portion 7310 according to an embodiment of the present disclosure. FIG. 50D is a perspective view of the second small dilator portion 7310.

The first small dilator portion 7300 may be configured to be assembled with the second small dilator portion 7310 to form a small dilator 7500. The first small dilator portion 7300 and the second small dilator portion 7310 may be configured to be injection molded. The first small dilator portion 7300 and the second small dilator portion 7310 may include features and draft angles conducive to injection molding.

The first small dilator portion 7300 may include a plurality of female interlocking features 7302, a cannulation portion 7304, a notch portion 7306, a tip portion 7308, and a grip portion 7309. The plurality of female interlocking features 7302 may include a combination of circular apertures and elongated slots. The plurality of female interlocking features 7302 may be generally equally spaced along a length of the first small dilator portion 7300. The plurality of female interlocking features 7302 may be configured to engage a plurality of male interlocking features 7312 of the second small dilator portion 7310.

The cannulation portion 7304 may be generally semi-circular and may extend along the length of the first small dilator portion 7300. The cannulation portion 7304 may not overlap or intersect with any of the plurality of female interlocking features 7302.

The notch portion 7306 may be generally rectangular and may extend along the length of the first small dilator portion 7300. The notch portion 7306 may not overlap or intersect with any of the plurality of female interlocking features 7302 or the cannulation portion 7304. The tip portion 7308 may be generally conical and/or tapered and may be configured to facilitate dilation of soft tissue. The grip portion 7309 may include grooves, notches, and/or a roughened surface to aid in handling and manipulation of the small dilator 7500.

The second small dilator portion 7310 may include a plurality of male interlocking features 7312, a cannulation portion 7314, a notch portion 7316, a tip portion 7318, and a grip portion 7319. The plurality of male interlocking features 7312 may include a combination of circular and elongated protrusions. The plurality of male interlocking features 7312 may be generally equally spaced along a length of the second small dilator portion 7310. The plurality of male interlocking features 7312 may be configured to engage the plurality of female interlocking features 7302 of the first small dilator portion 7300.

The cannulation portion 7314 may be generally semi-circular and may extends along the length of the second small dilator portion 7310. The cannulation portion 7314 may not overlap or intersect with any of the plurality of male interlocking features 7312.

The notch portion 7316 may be generally rectangular and may extend along the length of the second small dilator portion 7310. The notch portion 7316 may not overlap or intersect with any of the plurality of male interlocking features 7312 or the cannulation portion 7314. The tip portion 7318 may be generally conical and/or tapered and may be configured to facilitate dilation of soft tissue. The grip portion 7319 may include grooves, notches, and/or a roughened surface to aid in handling and manipulation of the small dilator 7500.

FIG. 51A is a perspective view of a small dilator 7500 according to an embodiment of the present disclosure. FIG. 51B is a top view of the small dilator 7500, FIG. 51C is a front view of the small dilator 7500, and FIG. 51D is a side view of the small dilator 7500. The small dilator 7500 may be configured to receive a k-wire 5800. The small dilator 7500 may be guided over the K-wire 5800 and inserted into a soft tissue of a patient to provide sufficient access to a facet joint. The small dilator 7500 may have a tip 7508 at a distal end and a grip portion 7518 at a proximal end. The small dilator 7500 may also have an outside diameter 7502 that may be slightly less than the inside diameter of a medium dilator 7520.

The small dilator 7500 may further include a cannulation 7504, a notch 7506, and a small dilator length 7516. The cannulation 7504 may result from the alignment of the cannulation portion 7304 and the cannulation portion 7314 during assembly of the small dilator 7500. The cannulation 7504 may have a generally circular cross-section and may extend the length of the small dilator 7500 so that a k-wire 5800 may pass through the entire length of the small dilator 7500.

The notch 7506 may result from the alignment of the notch portion 7306 and the notch portion 7316 during assembly of the small dilator 7500. The notch 7506 may have a generally rectangular cross-section and may extend the length of the small dilator 7500. The notch 7506 may be configured to slidably receive a locating feature 7523 of the medium dilator 7520.

The small dilator 7500 may further include a first axis 7510 and a second axis 7512. The first axis 7510 may pass through a center point of the outside diameter 7502 and may be aligned with a scam where the first small dilator portion 7300 connects to the second small dilator portion 7310. The second axis 7512 may also pass through the center point of the outside diameter 7502 and may be perpendicular to the first axis 7510.

A bottom surface 7507 of the notch 7506 may be parallel to the second axis 7512 and perpendicular to the first axis 7510. The cannulation 7504 may have a center point along the first axis 7510. Additionally, the center point of the cannulation 7504 may not be along the second axis 7512. The second axis 7512 may lie between the cannulation and the bottom surface 7507 of the notch 7506.

The cap 5102 may be eccentrically shaped with respect to the fastener 5104, as a result, it may be beneficial to dilate soft tissue in a corresponding eccentric profile. The small dilator 7500 may include a cannulation 7504 configured so that the small dilator 7500 may be advanced along a k-wire 5800 into the soft tissue of a patient. Additionally, a longitudinal axis of the cannulation 7504 may be parallel to but not coincident with a longitudinal axis of the small dilator 7500 so that, as the small dilator 7500 is advanced, the soft tissue may not be dilated equally in all directions relative to the k-wire 5800.

FIG. 52A is a perspective view of a medium dilator 7520 according to an embodiment of the present disclosure. FIG. 52B is a front view of the medium dilator 7520 and FIG. 52C is a bottom view of the medium dilator 7520. The medium dilator 7520 may be guided over the small dilator 7500 and inserted into a soft tissue of a patient to provide sufficient access to a facet joint. The medium dilator 7520 may have a tip 7528 at a distal end and a grip portion 7538 at a proximal end. The medium dilator 7520 may have an outside diameter 7522 that may be slightly less than the inside diameter of a large dilator 7540. The medium dilator 7520 may have an inside diameter 7524 that may be slightly greater than the outside diameter 7502 of the small dilator 7500.

The medium dilator 7520 may further include an inside diameter 7524, a notch 7526, a locating feature 7523, and a medium dilator length 7536. The inside diameter 7524 have a generally circular cross-section interrupted by the locating feature 7523. The inside diameter 7524 may extend the length of the medium dilator 7520 so that the small dilator 7500 may pass through the entire length of the medium dilator 7520.

The notch 7526 may have a generally rectangular cross-section and may extend the length of the medium dilator 7520. The notch 7526 may be configured to slidably receive a locating feature 7543 of the large dilator 7540.

The medium dilator 7520 may further include a first axis 7530 and a second axis 7532. The first axis 7530 may pass through a center point of the outside diameter 7522. The second axis 7532 may also pass through the center point of the outside diameter 7522 and may be perpendicular to the first axis 7530.

A bottom surface 7527 of the locating feature 7523 may be parallel to the second axis 7532 and perpendicular to the first axis 7530. The inside diameter 7524 may have a center point along the first axis 7530. Additionally, the center point of the inside diameter 7524 may not be along the second axis 7532. The second axis 7532 may lie between the center point of the inside diameter 7524 and the bottom surface 7527 of the locating feature 7523.

The medium dilator 7520 may include an inside diameter 7524 configured so that the medium dilator 7520 may be advanced along the small dilator 7500 into the soft tissue of a patient. Additionally, as the medium dilator 7520 is advanced, the soft tissue may not be dilated equally in all directions relative to the k-wire 5800 received in the small dilator 7500.

The locating feature 7523 of the medium dilator 7520 may cooperate with the notch 7506 of the small dilator 7500 to align the eccentricities of the small dilator 7500 and the medium dilator 7520, thereby dilating the soft tissue beneficially to implant the facet fixation device 5100.

FIG. 53A is a perspective view of a large dilator 7540 according to an embodiment of the present disclosure. FIG. 53B is a front view of the large dilator 7540 and FIG. 53C is a bottom view of the large dilator 7540. The large dilator 7540 may be guided over the medium dilator 7520 and inserted into a soft tissue of a patient to provide sufficient access to a facet joint. The large dilator 7540 may have a tip 7548 at a distal end and a grip portion 7558 at a proximal end. The large dilator 7540 may have an outside diameter 7542 that may be slightly less than the inside diameter of a cannula 7200. The large dilator 7540 may have an inside diameter 7544 that may be slightly greater than the outside diameter 7522 of the medium dilator 7520.

The large dilator 7540 may further include an inside diameter 7544, a notch 7546, a locating feature 7543, and a large dilator length 7556. The inside diameter 7544 have a generally circular cross-section interrupted by the locating feature 7543. The inside diameter 7544 may extend the length of the large dilator 7540 so that the medium dilator 7520 may pass through the entire length of the large dilator 7540.

The notch 7546 may have a generally rectangular cross-section and may extend from a proximal end of the large dilator 7540 to the grip portion 7558. Alternatively, the notch 7546 may extend the length of the large dilator 7540. The notch 7546 may serve as a visual indicator to aid in rotational alignment of the large dilator 7540 with the cannula 7200.

The large dilator 7540 may further include a first axis 7550 and a second axis 7552. The first axis 7550 may pass through a center point of the outside diameter 7542. The second axis 7552 may also pass through the center point of the outside diameter 7542 and may be perpendicular to the first axis 7550.

A bottom surface 7547 of the locating feature 7543 may be parallel to the second axis 7552 and perpendicular to the first axis 7550. The inside diameter 7544 may have a center point along the first axis 7550. Additionally, the center point of the inside diameter 7544 may not be along the second axis 7552. The second axis 7552 may lie between the center point of the inside diameter 7544 and the bottom surface 7547 of the locating feature 7543.

The large dilator 7540 may include an inside diameter 7544 configured so that the large dilator 7540 may be advanced along the medium dilator 7520 into the soft tissue of a patient. Additionally, as the large dilator 7540 is advanced, the soft tissue may not be dilated equally in all directions relative to the k-wire 5800 received in the small dilator 7500.

The locating feature 7543 of the large dilator 7540 may cooperate with the notch 7526 of the medium dilator 7520 to align the eccentricities of the medium dilator 7520 and the large dilator 7540, thereby dilating the soft tissue beneficially to implant the facet fixation device 5100.

The small dilator length 7516 may be greater than the medium dilator length 7536 which, in turn, may be greater than the large dilator length 7556. With a distal end of each of the small dilator 7500, the medium dilator 7520, and the large dilator 7540 aligned and proximate a portion of bone, the grip portion 7518, the grip portion 7538, and the grip portion 7558 may all be exposed and may be outside the surgical site. The exposed grip portions may allow the small dilator 7500, the medium dilator 7520, and/or the large dilator 7540 to be manipulated and/or removed in any sequence.

FIG. 54A is a perspective view of a cannula 7200 according to an embodiment of the present disclosure. FIG. 54B is a top view of the cannula 7200, FIG. 54C is a front view of the cannula 7200, and FIG. 54D is a side view of the cannula 7200. The cannula 7200 may be configured to be guided over the large dilator 7540 into the surgical site. After positioning the cannula 7200, the small dilator 7500, the medium dilator 7520, and the large dilator 7540 may be withdrawn, thus exposing the facet joint through the cannula 7200.

The cannula 7200 may include a body 7201 and a handle 7202. The body 7201 may have an inside diameter 7203 and a length 7206 along a longitudinal axis of the body 7201. The inside diameter 7203 may be slightly greater than the outside diameter 7542 of the large dilator 7540. The length 7206 may be less than the large dilator length 7556 so that, with the distal end of the large dilator proximate a surface of a bone and a distal end of the body 7201 also proximate the surface of the bone, the grip portion 7558 of the large dilator 7540 may be exposed outside of the body 7201.

The handle 7202 may include a notch 7204 proximate the body 7201 and an attachment feature located at an opposite end of the handle 7202. The notch 7204 may be used to rotationally align the cannula 7200 with the large dilator 7540, thereby facilitating proper orientation of the facet fixation device 5100 relative to a facet joint. The attachment feature 7205 may be used to attach the cannula 7200 to a table mount device that may be known in the art.

FIG. 55A is a side view of an inserter tube 5200 according to an embodiment of the present disclosure. FIG. 55B is a partial detail view of the inserter tube 5200 and FIG. 55C is an alternate side view of the inserter tube 5200. An inserter assembly 5500 may be used to implant the facet fixation device 5100 shown and described previously. The inserter assembly 5500 may include an inserter tube 5200, an inserter lockout 5300, and a removeable lock 5400. The inserter tube 5200 may include an inserter tube handle 5206 and a tube 5207. The tube 5207 may further include a slot 5201 and channels 5205, each through a wall of the tube 5207.

The slot 5201 may be configured to define a tab 5202. The tab 5202 may include a first locking prong 5203 and a second locking prong 5204. Further, the slot 5201 and the tab 5202 may be configured so that the tab 5202 may be deflected towards the inside of a tube 5207 when a force is applied to the first locking prong 5203 and/or the second locking prong 5204. Additionally, the slot 5201 and/or the tab 5202 may be configured so that the tab 5202 returns to its original position when the force is removed.

FIG. 56A is a side view of an inserter assembly 5500 according to an embodiment of the present disclosure. FIG. 56B is a partial detail view of the inserter assembly 5500 and FIG. 56C is an alternate side view of the inserter assembly 5500. The inserter assembly 5500 may include the inserter tube 5200 and an inserter lockout 5300. FIG. 57A is a front view of an inserter lockout 5300 according to an embodiment of the present disclosure. FIG. 57B is a top view of the inserter lockout 5300.

The inserter lockout 5300 may include an inserter lockout handle 5301 and arms 5302. Further, the inserter lockout handle 5301 may include a bevel 5304. Additionally, arms 5302 may include guide tabs 5303. The inserter lockout handle 5301 may be configured to interact with the first locking prong 5203 and the second locking prong 5204 such that, as the inserter lockout handle 5301 travels along a tube 5207, there may be two captive positions that may limit the movement of the inserter lockout 5300. The bevel 5304 may be configured to actuate the first locking prong 5203 and the second locking prong 5204 from a first captive position to a second captive position and from a second captive position to a first captive position. Each captive position may be overcome by applying a sufficient force on the inserter lockout handle 5301 along an axis of the tube 5207 in a desired direction.

FIG. 58A is a perspective view of the inserter assembly 5500 and FIG. 58B is a partial bottom detail perspective view of the inserter assembly 5500. The guide tabs 5303 may be configured to slidably connect with channels 5205. The channels 5205 and guide tabs 5303 may be configured so that the distance the guide tabs 5303 may travel within channels 5205 may be the same or greater than the distance between the first captive position and the second captive position of the inserter lockout 5300 relative to the inserter tube 5200. The inserter tube 5200 may further include cap locking tabs 5208. The cap locking tabs 5208 may have a generally rectangular profile and may include a lead-in angle and/or rounded corners. The cap locking tabs 5208 may be configured so that, when the inserter lockout 5300 is in the second captive position, the cap locking tabs 5208 may deflect outwards when the facet fixation device 5100 is inserted into the inserter tube 5200. The cap locking tabs 5208 may be further configured to engage the connection feature 5126 and connection feature 5128 in the cap 5102.

The inserter assembly 5500 may be further configured so that, when the inserter lockout 5300 is advanced to the first captive position, the arms 5302 may prevent the cap locking tabs 5208 from deflecting outwards. Additionally, the guide tabs 5303 may be configured to slidably engage an inner surface 5210 to prevent the arms 5302 from deflecting outwards. The inserter assembly 5500 and the facet fixation device 5100 may be configured so that when the cap locking tabs 5208 are engaged with the connection feature 5126 and the connection feature 5128, and the inserter lockout 5300 is in the first captive position, the facet fixation device 5100 may be captive relative to the inserter assembly 5500.

Additionally, the inserter tube 5200 may include a ledge 5209. The ledge 5209 may be configured as a depth stop for the facet fixation device 5100, or more specifically the cap 5102, within the inserter tube 5200. Moreover, the ledge 5209 and the cap 5102 may be configured so that when the cap 5102 is captive relative to the inserter assembly 5500, the ledge 5209 may contact a portion of the cap 5102 that may be solid material, e.g., a flared section 5150. This may enable insertion of the facet fixation device 5100 into a bony surface without damage to the first lattice structure 5180 and/or the second lattice structure 5175.

In another embodiment, an inserter tube 5200 may include a ridge 5221. The ridge 5221 may include an aperture 5215 configured to allow access to a drive feature 5165 in the fastener 5104 when the facet fixation device 5100 is engaged with the inserter tube 5200. Additionally, the inserter tube 5200 may include a recessed portion 5222 that may be configured to receive the cap 5102.

FIG. 59A is a perspective view of the inserter assembly 5500. FIG. 59B is a perspective view of the inserter assembly 5500 including a facet fixation device 5100. FIG. 59C is a perspective view of the inserter assembly 5500 including the facet fixation device 5100 according to an embodiment of the present disclosure. FIG. 59D is a perspective view of the inserter assembly 5500 including facet fixation device 5100 and a removeable lock 5400 according to an embodiment of the present disclosure. FIG. 60 is a top view of the removeable lock 5400 according to an embodiment of the present disclosure. FIG. 61A is a side view of an inserter assembly 5600 according to an embodiment of the present disclosure. FIG. 61B is a partial detail side view of the inserter assembly 5600. The removeable lock 5400 may include an inner surface 5401 that is configured to attach to the inserter tube 5200.

The inserter assembly 5600 may include a ridge 5601. The ridge 5601 may include an aperture 5602 configured to allow access to a drive feature 5165 in the fastener 5104 when the facet fixation device 5100 is engaged with the inserter assembly 5600. Additionally, the inserter assembly 5600 may include a recessed portion 5603 that may be configured to receive the cap 5102. The inserter assembly 5600 may further include an edge 5604 configured to engage a flared section 5150 of the cap 5102. The edge 5604 may limit the distance the cap 5102 may be seated within the recessed portion 5603. Moreover, the edge 5604 may enable insertion of the facet fixation device 5100 into a bony surface without damage to the first lattice structure 5180.

The recessed portion 5603 may be configured so that when the cap 5102 is fully seated within the recessed portion 5603 there is a first distance 5606 between the surface of the cap and the ridge 5601. Additionally, the recessed portion 5603 may be configured so that when the cap 5102 is fully seated within the recessed portion 5603 there is a second distance 5605 between a second portion 5160 of the fastener 5104 and the aperture 5116 of the cap 5102. The recessed portion 5603 may be configured so that the first distance 5606 is less than the second distance 5605 to prevent the fastener 5104 from disengaging with the cap 5102 during insertion of the facet fixation device 5100 into a bony surface, enabling rotation and linear translation between the fastener 5104 and the cap 5102.

FIG. 62A is a perspective view of a cap 7602 according to an embodiment of the present disclosure. FIG. 62B is a top view of the cap 7602, FIG. 62C is a side view of the cap 7602, and FIG. 62D is a front view of the cap 7602. The cap 7602 may share many of the characteristics of the cap 5102 and/or the cap 3102, which may be numbered similarly, including, beveled teeth 7630, a first end 7625, a second end 7627, and an indicator button 7640, among others. Similar features shown and described for cap 5102 and cap 3102 may be assumed to also apply to cap 7602.

The cap 7602 may further include a rotation axis 7616 and a rod portion 7605. A facet fixation device 5100 may include a fastener 5104 and the cap 7602. Alternatively, the cap 7602 may be implanted without a fastener.

The rod portion 7605 may be configured so that the cap 7602 may be rigidly coupled to other implants. More specifically, the cap 7602 may be rigidly coupled to a pedicle screw and rod construct. With the cap 7602 implanted in a first vertebra, the rod portion 7605 may be securely received in a tulip portion of a pedicle screw implanted in a second vertebra to form a rigid construct with a pedicle screw system.

The rod portion 7605 may be rotatably coupled to the rotation axis 7616 and may extend from the second end 7627 at an angle 7604 relative to a bone engaging surface 7628. The angle 7604 may be rotationally adjustable to be generally equivalent to a segmental lordosis angle and/or a disc lordosis angle to optimally couple with a pedicle screw. More specifically, the rod portion 7605 may be rotationally adjustable so that the angle 7604 may be between 10 degrees and 40 degrees.

The cap 7602 may further be configured to be coupled with a laminar hook. The rod portion 7605 may be received in a slot of the laminar hook and secured to the laminar hook with a fastener, thereby forming a rigid construct between the cap 7652 and the laminar hook.

The cap 7602 may be one of a set of differently-sized implants, each having a different rod length and/or a different rod diameter, wherein the rod length 7635 and/or the rod diameter 7637 may be optimized for different spinal levels.

FIG. 63A is a perspective view of a cap 7652 according to an embodiment of the present disclosure. FIG. 63B is a top view of the cap 7652, FIG. 63C is a side view of the cap 7652, and FIG. 63D is a front view of the cap 7652. The cap 7652 may share many of the characteristics of the cap 7602 and/or the cap 5102, which may be numbered similarly, including, beveled teeth 7680, a first end 7675, a second end 7677, and an indicator button 7690, among others. Similar features shown and described for cap 7602 and cap 5102 may be assumed to also apply to cap 7652.

The cap 7652 may further include an aperture 7666, a rod portion 7655 and a rod angle 7654. A facet fixation device 5100 may include a fastener 5104 and the cap 7652. Alternatively, the cap 7652 may be implanted without a fastener.

The aperture may be configured to receive a fastener 5104. The aperture 7666 may be offset from a geometric center of the cap 7652.

The rod portion 7655 may be configured so that the cap 7652 may be rigidly coupled to other implants. More specifically, the cap 7652 may be rigidly coupled to a pedicle screw and rod construct. With the cap 7652 implanted in a first vertebra, the rod portion 7655 may be securely received in a tulip portion of a pedicle screw implanted in a second vertebra to form a rigid construct with a pedicle screw system.

The cap 7652 may further be configured to be coupled with a laminar hook. The rod portion 7655 may be received in a slot of the laminar hook and secured to the laminar hook with a fastener, thereby forming a rigid construct between the cap 7652 and the laminar hook.

The rod portion 7655 may extend from the second end 7677 so that a longitudinal axis of the rod portion 7655 may be generally parallel to a longitudinal axis of the cap 7652. Additionally, the rod portion 7655 may extend at a rod angle 7654 relative to a bone engaging surface 7678. The rod angle 7654 may be generally equivalent to a segmental lordosis angle and/or a disc lordosis angle to optimally couple with a pedicle screw. More specifically, the rod portion 7655 extend from the cap 7652 so that the rod angle 7654 may be between 0 degrees and 40 degrees.

Alternatively, the rod portion 7655 may extend from a portion of the cap 7652 that may not be the second end 7677. Additionally, or alternatively, a longitudinal axis of the rod portion 7655 may not be parallel to a longitudinal axis of the cap 7652.

The cap 7652 may be one of a set of differently-sized implants, each having a different rod length and/or a different rod diameter, wherein the rod length 7685 and/or the rod diameter 7687 may be optimized for different spinal levels. Additionally, or alternatively, the cap 7652 may be one of a set of differently-sized implants, each having a different rod angle 7654, wherein the angle 7654 may be optimized for different spinal levels.

FIG. 64A is a perspective view of a facet fixation device 8000 according to an embodiment of the present disclosure. FIG. 64B is a top view of the facet fixation device 8000, FIG. 64C is a side view of the facet fixation device 8000, and FIG. 64D is a front view of the facet fixation device 8000. The facet fixation device 8000 may include a fastener 8100, a cap 5102, and a tulip 8200.

The facet fixation device 8000 may be configured to facilitate coupling of the facet fixation device 8000 with other implants. More specifically, the facet fixation device 8000 may be rigidly coupled to a pedicle screw and rod construct. The tulip 8200 may receive a first portion of a rod 8201 with a second portion of the rod 8201 coupled to a tulip of a pedicle screw implanted in a pedicle of a vertebra to form a rigid construct between the facet fixation device 8000 and a pedicle screw system.

The facet fixation device 8000 may be rigidly coupled to a laminar hook. The tulip 8200 may receive a rod portion of the laminar hook to form a rigid construct between the facet fixation device 8000 and the laminar hook.

The tulip 8200 may be poly-axially adjustable relative to the fastener 8100. A set screw 8202 may secure the rod 8201 to the tulip and may lock the orientation and/or angulation of the tulip relative to the fastener 8100. The cap 5102 may be received on the fastener 8100 distally from the tulip 8200. The facet fixation device 8000 may be configured so that a driver (not shown) may engage a drive feature 8107 of the fastener 8100 with the tulip 8200 captive on the fastener 8100.

The facet fixation device 8000 may further be configured so that the fastener 8100 may be inserted into a portion of bone to a depth sufficient to fully engage the beveled teeth 5130 of the cap 5102. With the cap 5102 engaged with the portion of bone, the tulip 8200 may be poly-axially moveable relative to the fastener 8100, and the tulip 8200 may receive the rod 8201. With the rod 8201 received in the tulip 8200, the tulip 8200 may receive a set screw 8202 configured to secure the rod 8201 to the tulip 8200 so that the rod 8201 is not moveable and/or rotatable relative to the tulip 8200, and the tulip 8200 is not moveable and/or rotatable relative to the fastener 8100.

FIG. 65A is an exploded perspective view of a facet fixation device 8000′ according to an embodiment of the present disclosure. FIG. 65B is a perspective view of the facet fixation device 8000′. The facet fixation device 8000′ may include the fastener 8100, the cap 5102, and a tulip 8200′. The tulip 8200′ may share most of the characteristics of the tulip 8200. Similar features shown and described for tulip 8200 may be assumed to also apply to tulip 8200′.

The facet fixation device 8000′ may be assembled by inserting a distal end 8103 of the fastener 8100 through a central aperture 8208′ of the tulip 8200′. The distal end may then be further advanced through the aperture 5116 of the cap 5102. The fastener 8100 may be further advance distally until the second portion 8110 of the fastener 8100 is proximate the aperture 5116 of the cap, and the poly-axial head 8104 of the fastener 8100 is proximate the poly-axial seat 8210′ of the tulip 8200′.

Alternatively, a tulip may be configured for top loading on the poly-axial head 8104 of the fastener 8100. In this embodiment, a facet fixation device may be assembled by inserting a distal end 8103 of the fastener 8100 through the aperture 5116 of the cap 5102. The fastener 8100 may be further advance distally until the second portion 8110 of the fastener 8100 is proximate the aperture 5116 of the cap. Then a distal end of the tulip may then be guided onto the poly-axial head 8104 of the fastener 8100. In this embodiment, the facet fixation device may be implanted as a fastener 8100 and cap 5102 assembly and the tulip may be guided onto the poly-axial head of the fastener 8100 after implantation of the fastener 8100 and cap 5102.

FIG. 66A is a perspective view of a fastener 8100 according to an embodiment of the present disclosure. FIG. 66B is a top view of the fastener 8100 and FIG. 66C is a front view of the fastener 8100. The fastener 8100 may be configured to engage a cap 5102 and a tulip 8200 as part of an implant construct. When implanted, fastener 8100 may extend through the cap 5102 and through a facet joint, fastening the cap 5102 to the facet joint and stabilizing the joint.

The fastener 8100 may include a threaded portion 8101, one or more cutting flutes 8102, a distal end 8103, a poly-axial head 8104, and a cannulation 8106 extending through the poly-axial head 8104 to the distal end 8103. A first portion 8112 may include a drive feature 8107 configured as a hex connection or other type of non-circular connection which may permit a driver to turn the fastener 8100. Additionally, or alternatively, the poly-axial head 8104 may include a drive feature 8107 configured as a hex connection or other type of non-circular connection which may permit a driver to turn the fastener 8100. The threaded portion 8101 may be configured with a cortical engaging thread form, a cancellous engaging thread form, or a combination of the two.

The one or more cutting flutes 8102 may be located at the distal end 8103 of the threaded portion 8101 and may be configured to interrupt the threaded portion 8101 to decrease the torque required to drive the fastener 8100 into a bone. The cannulation 8106 may be configured to receive a k-wire 5800 to aid in placement of the facet fixation device 8000.

The poly-axial head 8104 may be generally spherical and may be configured to be received in a poly-axial seat 8210 of the tulip 8200 to enable poly-axial movement of the tulip 8200 relative to the fastener 8100. A first portion 8112 and a second portion 8110 may be located between the poly-axial head 8104 and the threaded portion 8101. The second portion 8110 may have a larger diameter than the first portion 8112. The second portion 8110 may be configured to be received in the aperture 5116 of the cap 5102 but not pass through the aperture 5116, thereby enabling the fastener 8100 to secure the cap 5102 to the bone.

The poly-axial head 8104 may be configured so that the poly-axial head 8104 may break-away from the first portion 8112 if a tulip 8200 is not included in the implant construct. The break-away feature may be configured so that the poly-axial head 8104 may separate from the first portion 8112 when a pre-determined amount of torque is applied to the poly-axial head 8104. Alternatively, the break-away feature may be configured so that the poly-axial head 8104 may separate from the first portion 8112 when a pre-determined amount of linear force is applied to the poly-axial head 8104. The poly-axial head 8104 may be configured so that the poly-axial head 8104 may be separated from the fastener body before the fastener is implanted in a bone. Alternatively, the poly-axial head 8104 may be configured so that the poly-axial head 8104 may be separated from the fastener body after the fastener is implanted in the bone.

Alternatively, the fastener 8100 may be configured so that the poly-axial head 8104 may be a separate component configured to be assembled to the fastener body. The poly-axial head 8104 may be configured so that the poly-axial head 8104 may be secured to the fastener body after the fastener 8100 is implanted in the bone.

FIG. 67A is a side view of the tulip 8200 and FIG. 67B is a side section view of the tulip 8200. FIG. 68A is a perspective view of a tulip 8200 with a rod 8201 and a set screw 8202 according to an embodiment of the present disclosure. FIG. 68B is a top view of the tulip 8200 with the rod 8201 and the set screw 8202, FIG. 68C is a front view of the tulip 8200 with the rod 8201 and the set screw 8202, and FIG. 68D is a side view of the tulip 8200 with the rod 8201 and the set screw 8202.

The tulip 8200 may be configured to be received on the poly-axial head 8104 of the fastener 8100 such that the tulip 8200 may be poly-axially moveable relative to the fastener 8100. The tulip may further be configured to receive a rod 8201, thereby coupling the facet fixation device 8000 to a second implant construct.

The tulip 8200 may include a tulip thread 8204, a tulip body 8205, and a central aperture 8208. The tulip thread 8204 may be configured to threadably engage a set screw thread 8203 of a set screw 8202. The set screw 8202 may be configured to threadably engage the tulip 8200 to apply sufficient force to the rod 8201 to lock a position and/or rotation of the tulip 8200 relative to the fastener 8100 and lock a position and/or rotation of the rod 8201 relative to the tulip 8200.

The tulip body 8205 may include a tulip slot 8206 configured to receive the rod 8201. The tulip body may further include the central aperture 8208. The central aperture 8208 may include a poly-axial seat 8210 configured to receive the poly-axial head 8104 of the fastener 8100, thereby allowing poly-axial movement of the tulip 8200 relative to the fastener 8100. The central aperture 8208 and the poly-axial seat 8210 may be configured so that all portions of the fastener 8100 except the poly-axial head 8104 may pass through the tulip 8200.

In an embodiment, a method for implanting a facet fixation device, such as the facet fixation device 3100, may include the following steps:
1. Prepare surgical site and identify screw placement with bone access needle and/or k-wire
2. Insert dilator(s)
3. Insert a cannula;
4. Advance drill through cannula and over k-wire and drill hole into bony surface;
5. Remove the drill from the k-wire;
6. Advance reamer through cannula and over k-wire;
7. Remove the k-wire (if needed);
8. Ream surface;
9. Replace the K-wire (if removed);
10. Remove the reamer;
11. Set inserter assembly to second captive position;
12. Insert facet fixation device into inserter assembly;
13. Set inserter assembly to first captive position;
14. Attach removeable lock;
15. Insert inserter assembly/facet fixation device into cannula;
16. Engage screw driver with facet fixation device;
17. Tighten screw into bone;
18. Hammer for 2 mm advancement;
19. Tighten screw;
20. Hammer for further advancement;
21. Tighten screw;
22. Check indicator button;
23. Repeat steps 20 through 22 until indicator button indicates facet fixation device is fully seated against surface of bone;
24. Detach removeable lock;
25. Set inserter assembly to the second captive position;
26. Slide inserter assembly and screw driver out of cannula; and
27. Repeat for additional implants if needed.

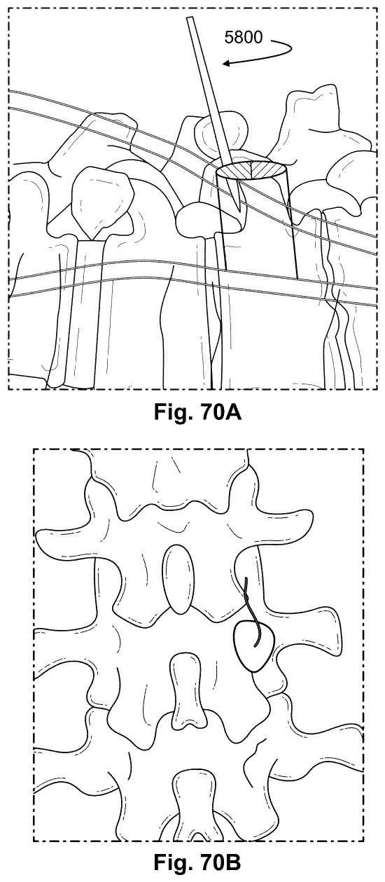

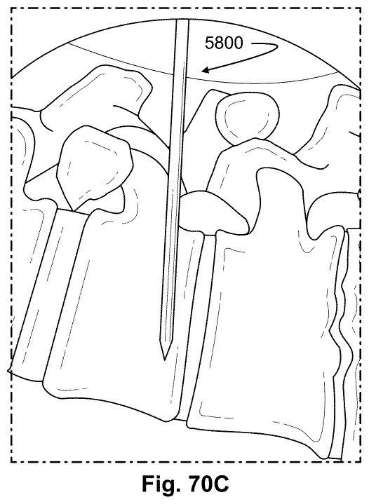

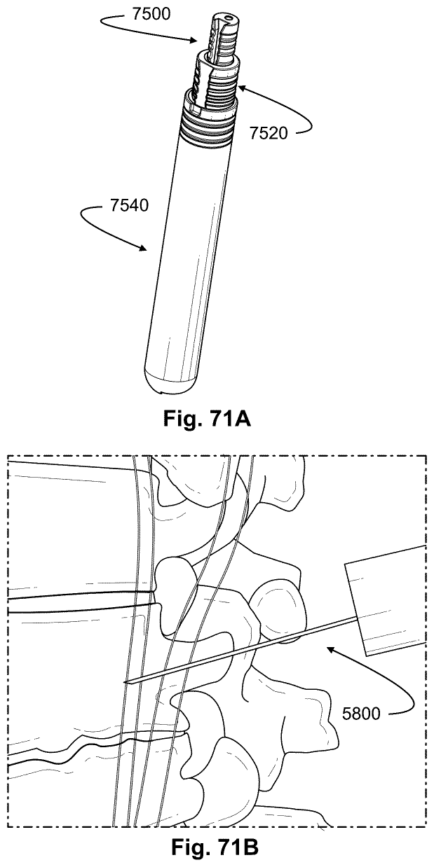

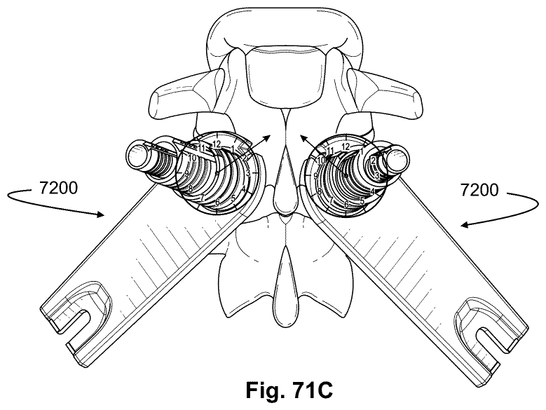

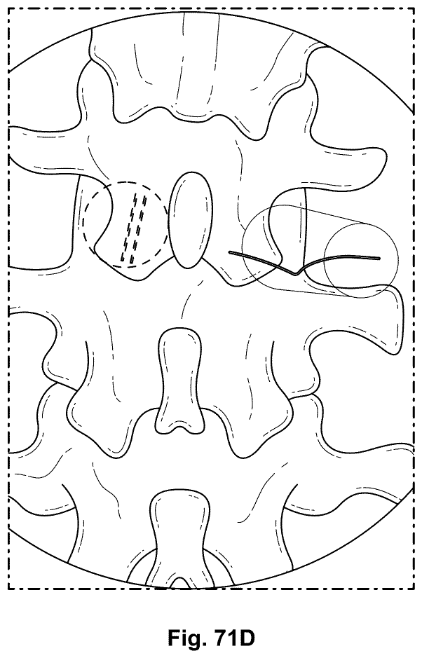

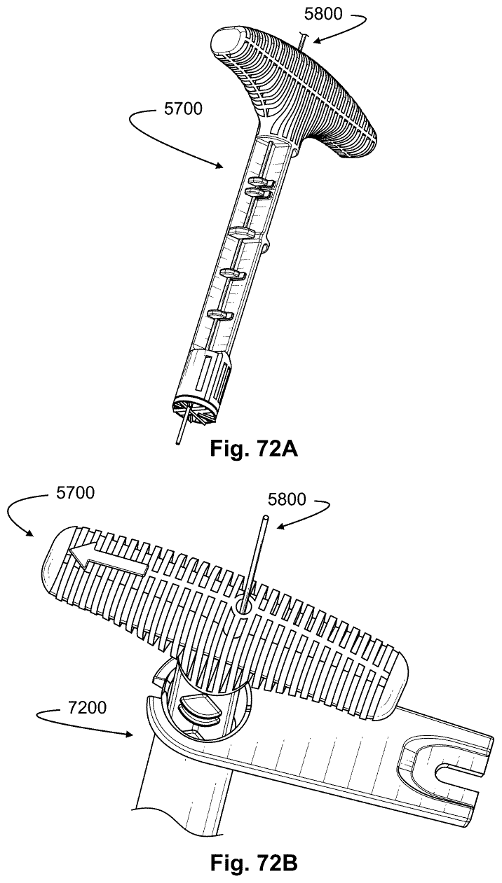

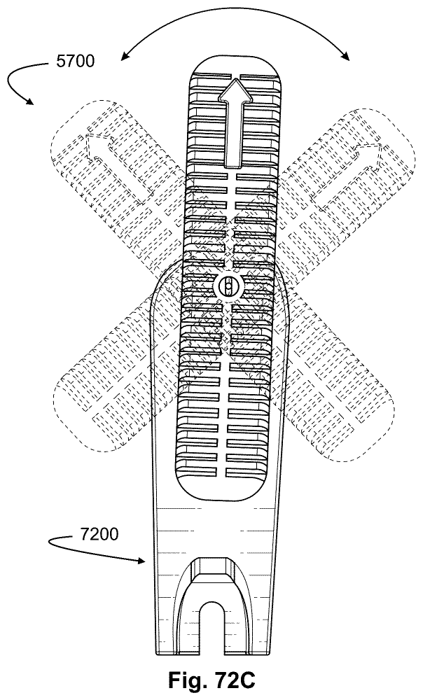

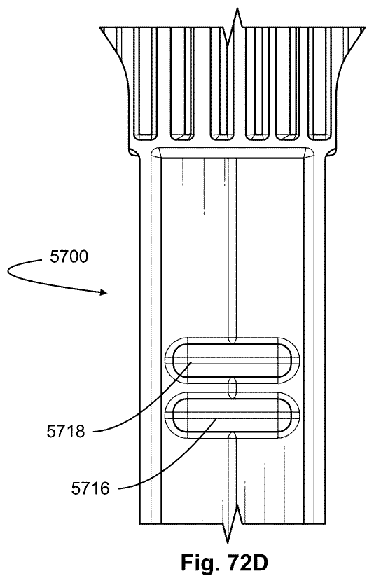

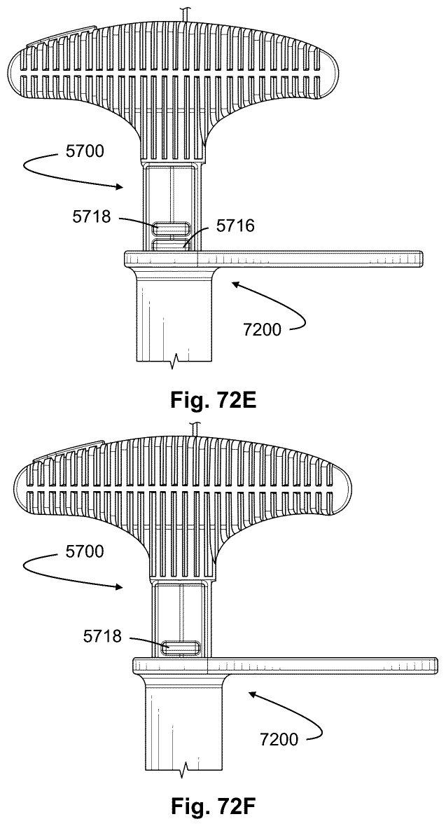

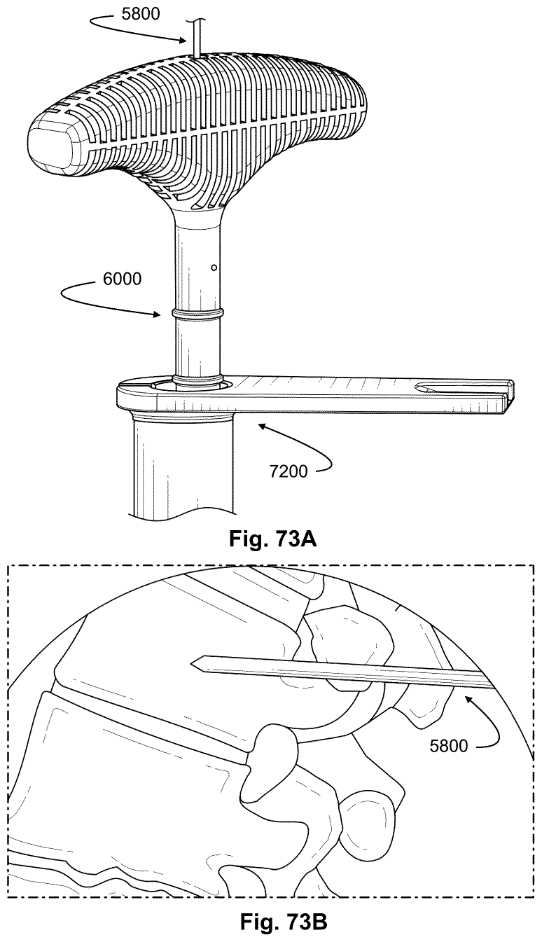

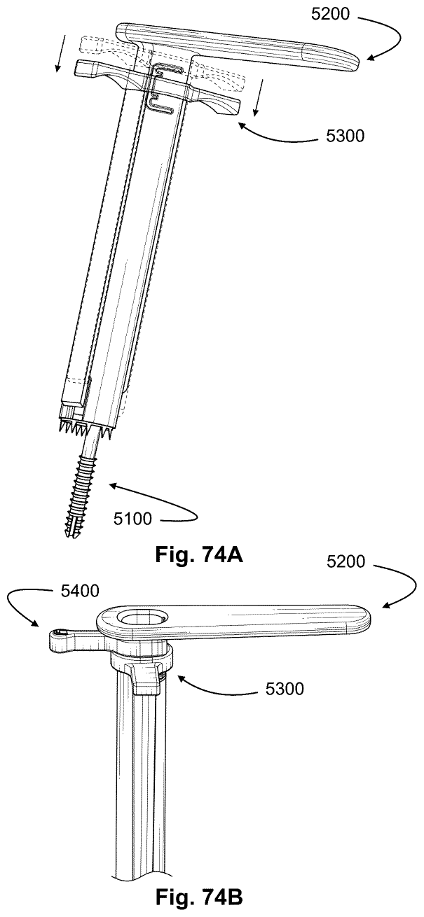

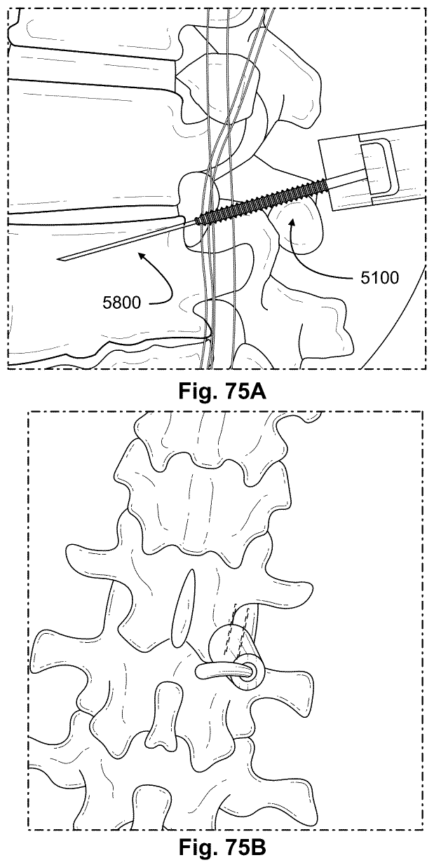

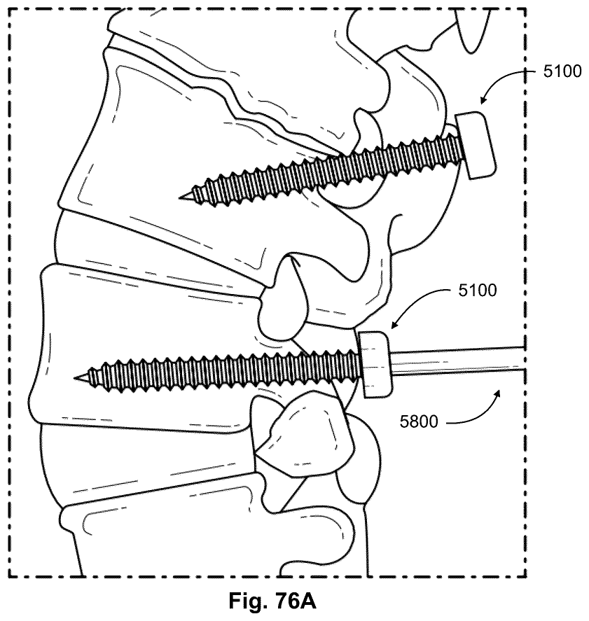

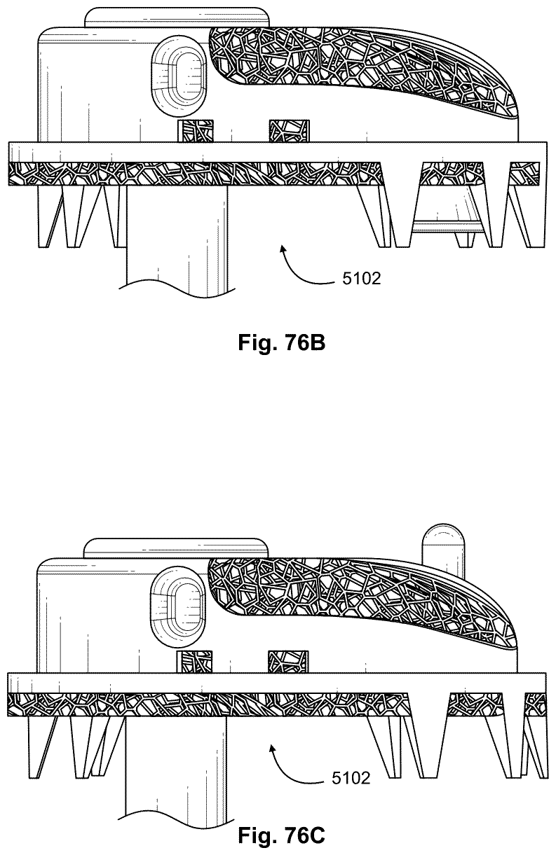

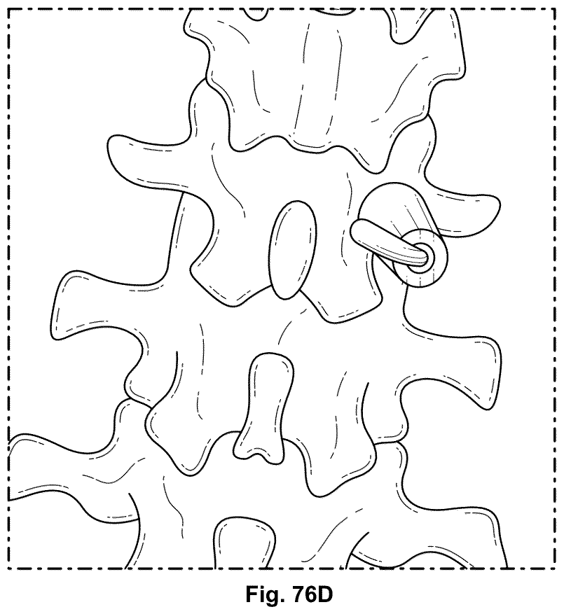

FIGS. 69A-77 are perspective views of steps in a method for facet fixation according to an embodiment of the present disclosure. In an embodiment, a method for implanting a facet fixation device, such as the facet fixation device 5100, may include the following steps:
1. FIG. 69A and FIG. 69B. Site planning and k-wire introduction:
   a. Identify and mark a midline.
   b. Acquire anterior-posterior (AP) imaging, confirm alignment with an index disc space.
   c. Identify inferior pedicle to the targeted disc space using AP and lateral imaging.
   d. Insert Jamshidi needle, targeting top ⅓ of the pedicle, just lateral to a facet joint.
2. FIG. 70A, FIG. 70B, and FIG. 70C. Site planning and k-wire introduction (continued):
   a. Insert Jamshidi into superior half of the pedicle and confirm trajectory will safely enter a vertebral body with lateral and AP imaging.
   b. Insert Jamshidi beyond the pedicle and into a proximal vertebral body.
   c. Confirm location of Jamshidi with AP image.
   d. Insert k-wire through Jamshidi.
   e. Remove Jamshidi.
   f. Cut skin and fascia (approx. 2 cm) centered on the k-wire.
3. FIG. 71A, FIG. 71B, FIG. 71C, and FIG. 71D. Dilation:
   a. Sequentially dilate using a small dilator, a medium dilator, and a large dilator while aligning notches while advancing the dilators.
   b. Insert a cannula over the dilators.
   c. Continue to advance the cannula to dock the cannula onto a facet.
   d. Rotate the cannula and the dilators until the notches are aligned and point at a:
      i. 2:00 o'clock on the patient's left
      ii. 10:00 o'clock on the patient's right
   e. Attach the cannula to a table mount, if desired.
   f. Remove the dilators.
   g. Use a Bovie (if needed) to expose the facet and remove overlying soft tissue.
   h. Acquire AP image and confirm visualization of facet joint.
4. FIG. 72A, FIG. 72B, FIG. 72C, FIG. 72D, FIG. 72E, and FIG. 72F. Ream:
   a. Visually inspect no overlying soft tissue and confirm no overlying soft tissue and confirm reamer will engage bone directly.
   b. Insert reamer over k-wire.
   c. Align an arrow on the reamer with a notch on the cannula.
   d. Ream by oscillating the reamer back and forth achieving approximately 45 degrees in both directions, or a quarter turn in total.
   e. Continue to ream until facet is sufficiently prepped and soft tissue is removed.
5. FIG. 73A and FIG. 73B. Drilling (if needed):
   a. Insert cannulated drill over k-wire
   b. Drill over the k-wire until the desired depth marker (35 mm or 50 mm) is in line with the top of the cannula. Confirm depth and location of drill tip with imaging.
6. FIG. 74A and FIG. 74B. Implant Insertion:
   a. Attach facet fixation device to inserter assembly and lock into place by pushing an inserter lockout distally one click.
   b. Attach removeable lock to inserter assembly.
   c. Insert a driver through the inserter assembly and into the fastener head.
7. FIG. 75A and FIG. 75B. Implant Insertion (continued):
   a. Insert facet fixation device over the k-wire.
   b. Insert the driver over the k-wire and drive the fastener until the cap is just above the facet.
   c. Rotate the inserter until the cap sufficiently spans the facet joint, confirm the cap placement with an AP image.
8. FIG. 76A, FIG. 76B, FIG. 76C, and FIG. 76D. Implant Insertion (continued):
   a. Continue to drive the fastener until force is too high, gently tap on the front of the inserter handle to seat the beveled teeth of the cap into the bone, continue the tap and screw process until the screw does not advance anymore with tapping.
   b. With lateral imaging, confirm facet fixation device is fully seated, indicator button may rise above the implant cap, indicating the implant is seated fully on the bone.
   c. Confirm the cap is bridging the facet joint with AP imaging.
9. FIG. 77. Remove instruments and Close:
   a. Remove driver and k-wire.
   b. Unlock inserter assembly from facet fixation device by removing the removeable lock and pulling proximally on the inserter lockout one click.
   c. Remove the inserter assembly.
   d. Disconnect the cannula from the table mount (if required) and remove.
   e. Close surgical site.

Those of skill in the art will recognize that this is only one of many potential methods of facet fixation. In alternative embodiments, the facet fixation device may be coupled with a pedicle screw construct. Additionally, or alternatively, multiple facet fixation devices may be implanted in multiple level of the spine. In yet other embodiments, different methods may be used for facet fixation other than the methods described above. Further, the methods set forth above may be used for facet fixation utilizing a facet fixation device besides those specifically disclosed herein.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “connected to,” “coupled to” and “in communication with” refer to any form of interaction between two or more entities, including mechanical, electrical, magnetic, electromagnetic, fluid, and thermal interaction. Two components may be rigidly coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

All changes which come within the meaning and range of equivalency of the claims are to be embraced within their scope.

1. A facet fixation system, the system comprising:
a cap configured to span a vertebral facet joint, the cap comprising a distal surface configured to seat against a bone proximate the vertebral facet joint; and
a fastener configured to secure the cap to the vertebral facet joint;
wherein the cap comprises an indicator configured to move from a first position to a second position in response to engagement of the distal surface with the bone.

2. The system of claim 1, wherein the cap and the indicator are integrally manufactured using an additive manufacturing process.

3. The system of claim 1, wherein the fastener and the cap are configured so that the fastener is captive relative to the cap.

4. The system of claim 1, wherein the cap comprises a plurality of recesses configured to receive a guiding instrument.

5. The system of claim 1, wherein the cap comprises a lattice structure configured to promote bone growth into the cap.

6. The system of claim 1, wherein the cap further comprises a rod portion configured to couple the facet fixation system with a pedicle screw system.

7. The system of claim 1, wherein the cap further comprises a rod portion configured to couple the facet fixation system with a laminar hook.

8. The system of claim 1, further comprising a tulip configured to receive a rod, wherein the rod rigidly couples the facet fixation system with a pedicle screw system.

9. The system of claim 1, further comprising a tulip configured to receive a rod portion of a laminar hook, wherein the rod portion rigidly couples the facet fixation system with the laminar hook.

1. The system of claim 1, further comprising a reamer configured to prepare a bone surface for implantation of a facet fixation device, the reamer comprising a cutting head comprising a plurality of cutting edges, wherein the cutting head is configured to cut in a clockwise direction and a counter-clockwise direction.

2. The system of claim 1, wherein the system further comprises a dilator configured to dilate soft tissue, the dilator comprising an inside diameter eccentric to an outside diameter.

3. The system of claim 1, wherein the fastener comprises a porous surface configured promote bone growth into the fastener.

4. The system of claim 1, wherein the fastener comprises a cylindrical screw head and the cap comprises an aperture, offset from a geometric center of the cap, configured to rotatably receive the cylindrical screw head.

14. A facet fixation system, the system comprising:
a cap configured to span a vertebral facet joint;
a fastener configured to secure the cap to the vertebral facet joint;
an inserter configured to engage the cap; and
a lock that is moveable, relative to the inserter, between a locked position, in which the cap is not disengageable from the inserter, and an unlocked position, in which the cap is disengageable from the inserter.

15. The system of claim 14, wherein the cap comprises a distal surface configured to seat against a bone proximate the vertebral facet joint and an indicator configured to move from a first position to a second position in response to engagement of the distal surface with the bone.

16. The system of claim 14, wherein the cap comprises a lattice structure configured to promote bone growth into the cap.

17. The system of claim 14, wherein the system further comprises a dilator configured to dilate soft tissue, the dilator comprising an inside diameter eccentric to an outside diameter.

18. The system of claim 14, further comprising a reamer configured to prepare a bone surface for implantation of a facet fixation device, the reamer comprising a cutting head comprising a plurality of cutting edges, wherein the cutting head is configured to cut in a clockwise direction and a counter-clockwise direction.

19. A facet fixation system, the system comprising:
a cap configured to span a vertebral facet joint, the cap comprising a distal surface configured to seat against a bone proximate the vertebral facet joint, and a proximal surface facing proximally; and
a fastener configured to secure the cap to the vertebral facet joint;
wherein the distal surface of the cap comprises a first lattice structure configured to promote bone growth into the cap.

20. The system of claim 19, wherein the proximal surface comprises a second lattice structure configured to promote bone growth into the proximal surface, wherein the second lattice structure is spaced apart from the first lattice structure.

21. The system of claim 20, wherein the cap comprises a hollow interior in communication with the second lattice structure and the first lattice structure such that bone growth can occur through the cap, from the second lattice structure to the first lattice structure, after implantation of the cap.

22. The system of claim 19, further comprising an inserter configured to engage the cap, wherein:
the cap comprises a load-carrying portion within which the first lattice structure is absent; and
the inserter is configured to engage the load-carrying portion to permit insertion of the cap into the bone without transmitting force through the first lattice structure.

23. The system of claim 22, wherein the inserter further comprises a recessed portion, wherein:
the recessed portion is configured to receive the cap and the fastener; and
the recessed portion is configured to prevent the fastener from disengaging from the cap during insertion of the cap.

24. The system of claim 19, wherein the system further comprises a dilator configured to dilate soft tissue, the dilator comprising an inside diameter eccentric to an outside diameter.

25. The system of claim 19, further comprising a reamer configured to prepare a bone surface, the reamer comprising a cutting head comprising a plurality of cutting edges, wherein the cutting head is configured to cut in a clockwise direction and a counter-clockwise direction.
